(19) United States
(12) Patent Application Publication
(10) Pub. No.: US 2022/0370529 A1
(43) Pub. Date: Nov. 24, 2022

(54) CANNABIS COMPOSITIONS AND RELATED METHODS

(71) Applicant: Access Business Group International LLC, Ada, MI (US)

(72) Inventor: Jesse C. LEVERETT, Petoskey, MI (US)

(21) Appl. No.: 17/731,636

(22) Filed: Apr. 28, 2022

Related U.S. Application Data
(60) Provisional application No. 63/185,822, filed on May 7, 2021.

(51) Int. Cl. A61K 36/185; A61K 9/00; A61K 47/02

(52) U.S. Cl. CPC A61K 36/185; A61K 9/0056; A61K 47/02; A61K 2236/17; A61K 2236/333

(57) ABSTRACT

A cannabis composition is disclosed. The cannabis composition comprises a cannabis extract, which comprises a cannabis phytocompound. A functional composition comprising the cannabis composition and a carrier is also provided, and adapted for administration to a subject via topical or oral application. A method of preparing the functional composition is also provided. A method of ameliorating a condition with the functional composition is further provided, and comprises administering the functional composition to a subject in an amount effective to elicit a biological response from the subject. The cannabis composition can be a multi-hemp extract composition. The multi-hemp extract composition generally comprises a first hemp component comprising a hemp dehydrate, a second hemp component different from the first hemp component, with the second hemp component comprising a hemp juice, and a third hemp component different from the first and second hemp components, with the third hemp component comprising a hemp extract.

### CROSS-REFERENCE TO RELATED APPLICATIONS

This application claims priority to and all advantages of U.S. Provisional Appl. No. 63/185,822, filed on May 7, 2021, the contents of which is incorporated herein by reference in its entirety.

### FIELD OF THE INVENTION

The present disclosure relates generally to functional compositions and, more specifically, to cannabis compositions useful for ameliorating conditions via form- and/or formulation-dependent administration to a subject, and methods relating to the same.

### DESCRIPTION OF THE RELATED ART

The endocannabinoid system (ECS) is an evolutionarily conserved network of molecular signaling that plays a role in bodily homeostasis. The ECS system is made up of multiple components, including: (a) signaling molecules called endocannabinoids: (b) specific receptors; and (c) enzymes that synthesize and breakdown endocannabinoids and transporters of endocannabinoids. The most well researched functions of the ECS are related to modulation of the central nervous system and immune function of the body. Recent research has indicated the critical role of the ECS in maintaining skin homeostasis and barrier function, and its dysregulation has been implicated in various skin disorders like atopic dermatitis, itch, acne, hair growth, pigmentation etc.

Various pathological states are associated with suboptimal functioning of ECS as a result of the altered levels of the endocannabinoids, their metabolizing enzymes, and the relative abundance of cannabinoid. For example, Alzheimer's disease, Parkinson's disease, Huntington's disease, multiple sclerosis, and epilepsy are examples of neurological disorders in which altered ECS function has been implicated. In addition, clinical endocannabinoid deficiency was suggested as the etiology of varied conditions including migraines, fibromyalgia, and irritable bowel syndrome. Additionally, suboptimal functioning of the ECS in the spinal cord has been associated with increased pain sensitivity. Accordingly, maintaining and supporting healthy ECS function may be necessary for optimal health.

CB1 receptors are generally present in abundance in the central nervous system (brain and nerves of the spinal cord) and CB2 are present in the peripheral nervous system (nerves in your extremities), the digestive system, and immune system. Research indicates that both CB1 and CB2 receptors are also found in epidermal keratinocytes, cutaneous nerve fibers, dermal cells, melanocytes, eccrine sweat glands and hair follicles. While cannabinoid receptors remain the primary targets for endocannabinoids, they have also been shown to bind to Transient Receptor Potential (TRP) receptors present in various types of skin cells and are involved in different functions like formation and maintenance of the skin barrier, cell growth, cell differentiation, immunological and inflammatory processes. In addition, endocannabinoids also interact with peroxisome proliferator-activated receptors (PPAR) via direct (endocannabinoid) or indirect (secondary metabolite of endocannabinoids) signaling pathways. PPAR (a and γ) activation partially mediates major biological functions of endocannabinoids like neuroprotection, anti-inflammation, and analgesic actions. These interactions have been best investigated for the endocannabinoids PEA and OEA, which activate PPARα. However, there are many aspects of PPAR and ECS interactions that have yet to be investigated.

Because of the importance of the ECS to overall health outcomes, supporting the appropriate functioning of the ECS could be a relevant therapeutic goal in varying treatments and methods relating to optimal biological functions. Several synthetic modulators of ECS have been used and studied for a broad range of diseases; however, they have introduced serious unexpected complexities. Therefore, there is an ongoing need for identifying methods of enhancing the ECS to treat or ameliorate the symptoms of these diseases without drugs or medical procedures. Recent research indicates that nutritional therapies using naturally occurring cannabinoids found in foods, such as plant-derived cannabinoids and cannabimimetics (i.e., “phytocannabinoids”), may be promising candidates due to high safety and low adverse effects profiles.

### BRIEF SUMMARY OF THE INVENTION

A cannabis composition is provided. The cannabis composition comprises a cannabis extract, which comprises a cannabis phytocompound. In some embodiments, the cannabis phytocompound comprises, alternatively is, cannabidiol (CBD).

A functional composition is also provided. The functional composition comprises the cannabis composition and a pharmaceutically acceptable carrier. In certain embodiments, the functional composition is in the form of a food or supplement (e.g. an oral and/or dietary supplement). In other embodiments, the functional composition is in the form of a personal care product (e.g. a topical preparation).

In various embodiment, the cannabis composition is a multi-hemp extract composition. The multi-hemp extract composition comprises a first hemp component comprising a hemp dehydrate, a second hemp component different from the first hemp component, the second hemp component comprising a hemp juice, a third hemp component different from the first and second hemp components, the third hemp component comprising a hemp extract, and a carrier component different from each of the hemp components.

In various embodiments, the hemp dehydrate is obtained from at least one of Cannabis leafs, buds, and flowers. In addition, the hemp juice is obtained from aerial portions of immature Cannabis plants. Moreover, the hemp extract is obtained from solvent extraction of at least one of Cannabis buds and flowers.

A method of preparing the functional composition is further provided. The method comprises combining the cannabis composition with the pharmaceutically acceptable carrier, thereby preparing the functional composition. In certain embodiments, the cannabis composition is in the form of a CBD isolate.

A method of ameliorating (e.g. treating, preventing) a condition with the functional composition is also provided. The method comprises administering the functional composition to a subject in an amount effective to elicit a biological response from the subject. In certain embodiments, the method comprises administering the functional composition topically to the subject, e.g. in the form of the personal care product. In other embodiments, the method comprises administering the functional composition enterally to the subject, e.g. in a consumable form such as the oral and/or dietary supplement.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and practiced or carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### DETAILED DESCRIPTION OF THE INVENTION

The present disclosure provides a cannabis composition. In general, the cannabis composition comprises a cannabis extract, i.e., a substance obtained from Cannabis plant material via extraction or related techniques. The cannabis extract comprises one or more cannabis phytocompounds, which generally include cannabinoids, terpenoids, and/or flavonoids that may be extracted or otherwise derived from the Cannabis plant. The cannabis extract may be enriched with one or more desired cannabis phytocompounds, e.g. relative to the concentration of such cannabis phytocompounds in the Cannabis material. Additionally, the cannabis extract may contain a lower concentration of unwanted material, including certain phytocompounds (e.g. particular terpenes, alkaloids, hemp oil, waxes, cannabinoid acids), as compared to the Cannabis plant material.

In some embodiments, the cannabis composition is formulated as a functional composition, such as a supplement (i.e., a consumable composition) or a topical composition (i.e., a personal care product). For example, the cannabis extract may be formulated with any convenient pharmaceutically acceptable diluents, carriers, excipients, etc. to prepare the consumable composition. The choice of diluents, carriers, excipients, etc. will depend on a desired dosage form of the consumable composition, which may in turn be dependent on the intended route of administration to a subject. For example, such dosage forms include liquid dosage forms (e.g. for administration via pump-action or aerosol sprays), tablets, pastilles, gels, capsules, suppositories, powders, etc. and vaporizers. Likewise, the consumable composition as a whole may be in the form of a consumable or “edible” product in various forms such as gummies, chews, shots, bars, barks, etc., which will generally be understood to include standard food and snack items compatible with the cannabis extract (i.e., capable of including the cannabis extract as a component/ingredient therein). Such dosage forms may be prepared in accordance with standard principles of pharmaceutical and/or supplement formulation known to those skilled in the art. The cannabis extract may also be formulated with an acceptable carrier to prepare the topical composition. The choices of carrier will also be selected in view of a desired end-use or application of the topical composition. For example, the topical composition may be formulated as a cream, gel, powder, paste, or freely pourable liquid, e.g. depending on a desired route of application to subject/consumer. These and other embodiments will be understood in view of the description below.

### Cannabis Composition

A cannabis composition is provided. The cannabis composition comprises a cannabis extract.

### Cannabis Extract

As used herein in the context of the cannabis composition and related embodiments, the term “extract” is to be understood in the ordinary and customary sense, i.e., as a substance obtained by extracting a raw material using a solvent system or equivalent process. Examples of extracting processes (i.e., “extractions”) include hot solvent extractions, supercritical fluid extractions (SFE) such as fractional supercritical fluid extraction (FSFE), etc. In this same context, the term “cannabis” is used herein to refer generally to plants of genus Cannabis spp., which is a member of family Cannabaceae and includes the herbaceous flowering plant species Cannabis sativa. As such, the term “cannabis extract” refers to a substance generally obtained via extraction of a cannabis material (i.e., a material comprising or consisting of at least a part of a Cannabis sativa plant). However, it will be appreciated from the description below that the substance of the cannabis extract is to be considered broadly, and encompasses forms that may be more specifically described as “isolates,”, “distillates,” “concentrates,” etc. As such, while the term “extract” may possess a specific meaning with regard to a particular cannabis product, it is to be understood that, in the context of the disclosure herein, the term “cannabis extract” is to be interpreted broadly in view of the description and examples below.

### Cannabis

As will be understood by those of skill in the art, the species Cannabis sativa is typically classified into three taxa/subspecies: Cannabis sativa, Cannabis indica, and Cannabis ruderalis. For purposes of this disclosure, the cannabis extract is not especially limited in terms of being prepared with any particular Cannabis spp., or cultivar, strain, or variety, hybrid, or landrace thereof. Rather, in some embodiments, the cannabis extract may be prepared from material from any such Cannabis plant. In certain embodiments, the cannabis extract is prepared from material from a Cannabis plant known as sativa-dominant (e.g., Charlotte's Web, Diesel, Haze, Jack Herer, Shaman, Skunk, Sour, Te Puke Thunder, etc.), indica-dominant (e.g., Blueberry, BC Bud, Holland's Hope, Kush, Northern Lights, Purple, White Widow, etc.), pure sativa (e.g., Acapulco Gold and Malawi Gold (i.e., Chamba), etc.), and the like, or derivatives, modifications, and/or combinations thereof. It will be appreciated that unique Cannabis varietals, cultivars, hybrids, etc. may be utilized in the preparation of the cannabis extract, e.g. to provide specific properties to the cannabis extract, to increase the ease of preparing the cannabis extract, to impart unique characteristics to the cannabis composition, etc., as will be understood in view of the description of these processes and compositions below. As such, in some embodiments, the cannabis extract is prepared from but one cultivar of Cannabis sativa. In this context, the term “cultivar” refers to a group of similar plants that can be identified from other varieties within the same species by structural features and performance (i.e., morphological and physiological characteristics). Likewise, the term “cultivar” may refer to a variety, strain, or race of plant that has been produced by horticultural or agronomic techniques, and is otherwise not normally found in wild populations. That said, it will be appreciated that the terms “cultivar,” “variety,” “strain,” and race are often used interchangeably by plant breeders, agronomists, and farmers. In other embodiments, the cannabis extract may be prepared from more than one cannabis cultivar.

The cannabis extract can be prepared from any cannabis material, such as from a leaf, stem, bud, flower, trichome, seed, root, etc., or combinations thereof. These terms will be understood by those of skill in the art to have the conventional botany definitions used for descriptive plant morphology. In particular, the term “leaf” refers to an organ of a vascular plant. In a Cannabis plant, the first pair of leaves typically have a single leaflet, with the number of leaflets gradually increasing up to a maximum of about thirteen leaflets per leaf, and typically seven or nine, depending on variety and growing conditions. The term “bud” refers to a flower-bearing stem or branch of the Cannabis plant, especially a stem or branch bearing a mass of female flowers (and associated leaves). The term “trichome” refers to a fine outgrowth or appendage on plants and certain protists (e.g. hairs, glandular hairs, scales, and papillae), which present and function in various ways. Cannabis presents a glandular trichome, which occurs most abundantly on the floral calyxes and bracts of female plants. The term “seed” refers to an embryonic plant enclosed in a protective outer covering (i.e., the “seed coat”), usually with some stored nutrients.

The cannabis extract can be prepared from any part or parts of cannabis materials, which may be used in a processed or raw/unprocessed form. For example, stems or branches bearing the female flowers can be fresh or dried, buds can be trimmed, cleaned, etc. In this context, the term “fresh” refers to a cannabis plant of part thereof (e.g. a leaf, flowering bud, seed, etc.) harvested within 21 days of being used in the extraction. In specific embodiments, a fresh cannabis material will have been harvested within 14 days of the extraction, such as within 10 days, alternatively within 7 days, alternatively within 3 days. In certain embodiments, the fresh cannabis material utilized will have been harvested within 15 days prior to anticipated harvest and/or in accordance with guidelines or rules provided by a particular governing organization such as the United States (U.S.) Department of Agriculture (USDA). As used herein, the term “dried” in the context of the cannabis material refers to a part or parts of cannabis material that has undergone a drying process (e.g. including elevated temperature, decreased pressure, etc.), such that a substantial and significant amount of water (or moisture) has been removed. For example, dried cannabis material may have a moisture content of less than 10 wt. %, alternatively less than 5 wt. %, alternatively less than 1 wt. %.

General examples of processes that may be used to prepare cannabis material for extraction (i.e., pre-extraction processing) include cutting, grinding, soaking, blending, sifting, milling, drying, desiccating, and the like, as well as combinations (e.g. sequential use) thereof. In certain embodiments, a chemical process, such as decarboxylation (e.g. via heating), protonation (e.g. via acidification), deprotonation (e.g. via basification), and the like, may be used to modify one or more substances in the cannabis material in order to increase or alter the efficacy of a subsequent extraction step. Likewise, various purification techniques, such as those described below (e.g. filtration, chromatography, etc.), may be utilized with such processes (e.g. before and/or after individual steps or sequences).

In certain embodiments, the cannabis extract can be prepared from a substance physically derived from a Cannabis plant, such as hashish, kief, rosin, etc., which are to be understood as also falling within the definition of the cannabis material as used herein. For example, the term “kief” refers to the resin glands (or trichomes) of Cannabis, which may accumulate in containers or be sifted from loose dry Cannabis flower (e.g. with a mesh screen or sieve). The term “hashish” refers to a compressed or purified preparation of stalked trichomes. One of skill in the art may select a particular cannabis material, e.g. based on a particular form of the cannabis extract and/or cannabis composition, as described further below. For example, it is generally known that kief typically contains a higher concentration of psychoactive cannabinoids, described below, than the Cannabis flowers from which it is derived. Similarly, hashish typically contains the same cannabinoids, but in higher concentrations, over unshifted buds or leaves.

The form of the cannabis extract is not particularly limited, and will be selected by those of skill in the art, e.g. based on a particular source and/or method used in its preparation, based on a particular type of formulation of the cannabis composition, etc. As such, the cannabis extract may be a direct extract, such as a solvent-based composition obtained from an extraction process or, alternatively, a processed form of such an extract (e.g. prepared via removal of solvent and/or additional processing/formulation steps. In this fashion, the cannabis extract may be further defined, or otherwise characterized, as an oil or essential oil, a tincture, a concentrate, an isolate, a distillate, a raffinate, or the like, or combinations thereof. Such characterizations may be based on colloquial or common usage of the terms, based on art-specific usage of the terms, or both, e.g. where a “tincture” in this context may refer generally to a solvent extract of plant material, a solution of such an extract, and/or of a low volatility substance obtained from such an extraction. Similarly, the terms “concentrate” and “essential oil” may both be used in the context of the cannabis extract to refer to a substance obtained by extracting a cannabis material with using a solvent, and subsequently removing, or mostly removing the solvent. In certain instances, the cannabis extract may be defined or otherwise characterized based on a regulatory definition. For example, in certain embodiments the cannabis extract is a “botanical drug substance” as defined in the Guidance for Industry Botanical Drug Products Draft Guidance, August 2000, prepared by the U.S. Department of Health and Human Services (HHS), Food and Drug Administration (FDA) Center for Drug Evaluation and Research (CDER), which provides the following definition: “A drug substance derived from one or more plants, algae, or macroscopic fungi. It is prepared from botanical raw materials by one or more of the following processes: pulverization, decoction, expression, aqueous extraction, ethanolic extraction, or other similar processes.”

It will be understood that certain forms of the cannabis extract, such as those introduced herein, may overlap with one another with regard to production methods, state-specific properties, intermediate forms, etc. For example, a cannabis tincture prepared via hot solvent extraction of cannabis material may be concentrated or distilled to prepare a cannabis concentrate or essential oil. Likewise, a cannabis tincture may also be prepared using cannabis isolates in conjunction with a carrier oil, such as olive oil, medium-chain triglycerides (MCT oil), etc. As will be understood in the art, the term “hash oil” refers to such a substance, i.e., prepared via extraction and subsequent removal of the extracting solvent. Hash oil is a resinous matrix, typically presenting as a hardened or viscous mass once concentrated a sufficient amount. Depending on the particular extraction parameters and cannabis material, hash oil may be a highly-potent form of the cannabis extracts in terms of active concentrations. For example, many conventional hash oils are prepared with a very high content of tetrahydrocannabinol (THC), which is described in further detail below. As such, a hash oil may be used directly in the cannabis composition or, alternatively, be used as an intermediate in the isolation of a particular compound or set of compounds (i.e., via purification, such as fractional distillation, chromatography, etc.). A similar form is that of a cannabis rosin prepared via solventless extraction involving the application of heat and pressure to express a resinous product from a cannabis material (e.g. from within trichome glands).

In certain embodiments, the cannabis extract is prepared via one or more post-extraction processes, which may be characterized as a purification process. In the context of the cannabis extract, such purification processes may be employed to remove or reduce a substance from the composition being purified, or remove a desired substance (i.e., the cannabis extract) from a mixture of substances obtained from the cannabis material. Examples of substances that may be removed include waxes, pigments, oils, terpenes, and even certain cannabinoids, as described in further detail below. Example of purification techniques that may be employed include dewaxing, distillation, concentration, filtration, chromatography, fractionation, isolation, secondary extraction, and the like, as well as combinations thereof. In certain embodiments, a chemical process, such as decarboxylation (e.g. via heating), protonation (e.g. via acidification), deprotonation (e.g. via basification), and the like, may be used to modify one or more substances in a post-extraction intermediate in order to increase or alter the efficacy of a subsequent purification step. In this fashion, it will be appreciated that the cannabis extract can be selectively enriched with particular phytochemicals (e.g. cannabinoids, terpenoids, and/or flavonoids) from the cannabis material utilized in the extraction. Likewise, the cannabis extract may contain lower concentrations of any such phytochemicals that are undesirable in a particular formulation final cannabis composition. Depending on the particular end use of the cannabis composition, as described below, such phytochemicals may include terpenes, alkaloids, and cannabinoid acids.

In some embodiments, the cannabis extract may be defined in terms of the amount of a specific active compound, or set of such compounds, therein. For example, as will be understood by those of skill in the art with regard to the cannabinoid cannabidiol (CBD), which is described in further detail below, the term “isolate” is used with reference to a pure form of CBD, i.e., a purified cannabis extracts comprising 99% CBD (e.g. by weight). As such, the term “isolate” may be applied to a cannabis extract consisting essentially of but one cannabis phytochemical, or only a limited number of cannabis phytochemicals, such as those described further below. In particular, the cannabis extract may be defined as an isolate (i.e., the “cannabis isolate”) when comprising a content of ≥99% of a single phytochemical, such as a phytocannabinoid, terpenoid, etc. In some embodiments, the term “isolate” in this context may also extend to extracts comprising ≥98, alternatively ≥95% CBD by weight. In the same fashion, the term “distillate” is used with reference to less-pure, but still purified form of CBD, e.g. comprising ≥80% CBD. Such terms and descriptions may be equally applied to other particular phytochemicals described herein, such as the specific phytocannabinoids described in further detail below.

In certain embodiments, cannabis isolates and distillates comprise minimal amounts of other phytochemicals in the same class as the named-compound. A typical CBD isolate, for example, will comprise levels of THC (i.e., another phytocannabinoid) below the limit of quantitation (<LOQ) or limit of detection (<LOD) when analyzed via an approved or otherwise generally-accepted method in the relevant industry. Such LOQs may be in the range of 0.001 to 0.4% (e.g. up to 0.1% or 0.39%, based on the specifics of the method involved), with more sensitive analyses in the lower end of the range (e.g. having an LOQ of from 0.001 to 0.01%, depending on the compound of interest). Likewise, a CBD distillate may comprise a purity of ≥80% CBD, but may also comprise a THC content <LOQ, or even <LOD.

In some embodiments, the cannabis extract may be defined as “full-spectrum,” which term refers to the presence of a wide variety of other cannabis phytochemicals. For example, in this context, a full-spectrum CBD extract may comprise numerous other phytocannabinoids, terpenes, etc. (i.e., multiple types/species of compounds from the “full spectrum” of those present in cannabis material). Such full-spectrum extracts may be prepared via less-selective extraction methods, or instead via combining different extraction products to reach a desired composition (e.g. in terms of content, ratios, or types of phytochemicals therein). Full-spectrum may also be defined as being a phytochemically complete extract, such as being composed of the entire range of soluble compounds present in cannabis. Such full-spectrum extracts can be made through exhaustive extractions using both hydrophilic and hydrophobic solvents, or also by adding in crude material to an extract, as will be understood in view of the description herein.

In some embodiments, the cannabis extract may be defined as “compliant” or “full-spectrum compliant” when meeting certain regulatory standards concerning THC content. For example, the US Agriculture Improvement Act of 2018 (i.e., the “2018 Farm Bill”) defines hemp as the plant species Cannabis sativa L. and any part of that plant, including the seeds thereof and all derivatives, extracts, cannabinoids, isomers, acids, salts, and salts of isomers, whether growing or not, with a THC content of not more than 0.3 percent on a dry weight basis (i.e., as calculated per an applicable standard, such as those published by the USDA as Laboratory Testing Guidelines under the U.S. Domestic Hemp Production Program). Likewise, the definition of “marihuana” in the US Controlled Substances Act (CSA) (i.e., Title 21 of the United States Code), also includes parts of the cannabis plant, and derivatives thereof, containing more than 0.3 percent THC on a dry weight basis. As these provisions of US Federal law remain in effect for purposes of Federal criminal prosecutions and civil and administrative proceedings arising under the CSA, the term “compliant” is associated in US markets and related industries with being a “hemp” product (i.e., having a THC level of 0.3 wt. % percent or less) or a “marijuana” or “marihuana” product (i.e., having a THC level exceeding 0.3 wt. %), with the later remaining classified in the US as a Schedule I controlled substance regulated by the US Drug Enforcement Administration (DEA) under the CSA.

### Phytocompounds

The cannabis extract comprises, alternatively consists essentially of, one or more phytocompounds obtained or otherwise derived from the cannabis material. As will the understood by those of skill in the art, phytocompounds, which may also be referred to generally as “phytochemicals,” are chemical entities that are found in or derived from plants, and thus represent a diverse group of substances that vary widely in terms of structure, form, and biological activity in non-plant organisms. For example, certain phytocompounds may be classified as macromolecular, polymeric, low molecular weight, or small molecules. Many phytocompounds are not bioactive with respect to certain biological processes, e.g. due to being non-digestible, poorly bioavailable, and/or otherwise recalcitrant or inert with respect to particular biological targets. Other phytocompounds, however, are bioactive and may therefore be exploited in functional compositions, as described in further detail below. In certain instances, phytocompounds presenting identified biological activity are further defined or characterized as “phytonutrients,” due to demonstrated positive impacts on physiological functions in humans, or even as “essential nutrients” when the phytocompound is required for normal physiological function and must therefore be obtained dietarily. Other specific classifications of phytocompounds are also known in the art and based upon a particular characteristic or property, such as phytotoxins (i.e., phytocompounds toxic to humans and/or other species), antinutrients (i.e., phytocompounds that interfere with the absorption of nutrients in a host), etc. Owing to such diverse activity and related effects, and as described in further detail below, the specific phytocompounds, or limited group of phytocompounds, in the cannabis extract may be advantageously employed in various ways that are not practical, effective, or even possible with the cannabis material from which they are obtained.

In general, phytocompounds that may be present in the cannabis extract include cannabinoids, terpenes, terpenoids, flavonoids, phenolics, glycosides, and alkaloids, as well as derivatives thereof, as will be appreciated in view of the further description and examples herein.

### Phytocannabinoids

In certain embodiments, the cannabis extract comprises one or more cannabinoids. Cannabinoids are a class of diverse chemical compounds that activate or otherwise interact with cannabinoid receptors, and can be further classified into three general categories based on origin. Specifically, phytocannabinoids (PCBs) are prepared by/obtained from plants, endocannabinoids (ECBs) are biosynthesized within the human body, and synthetic cannabinoids (SCs) are man-made chemicals prepared synthetically (e.g. via organic synthesis and/or bioorganic synthesis techniques). While the cannabis composition may comprise any one or more of such cannabinoids, for the purposes of this disclosure it is to be appreciated that the cannabinoids of the cannabinoid extract are exemplified generally by phytocannabinoids and, more specifically, by phytocannabinoids obtainable from the cannabis material (e.g. via extraction).

Over 100 unique cannabinoids have been extracted from Cannabis plants, inclusive of isomers and non-biosynthetically produced natural derivatives (e.g. oxidative degradants) of corresponding biosynthetically-formed analogs. These phytocannabinoids are generally derived from decarboxylative cyclization of 2-carboxylic acids, and typically classified based on particular features of the cyclic structure formed. Examples of such cannabis phytocannabinoids include those classified as cannabigerol-type, cannabichromene-type, cannabidiol-type, tetrahydrocannabinol-type, cannabinol-type, cannabielsoin-type, iso-tetrahydrocannabinol-type, cannabicyclol-type, and cannabicitran-type. As will be understood in view of the description herein, each of these types/classifications includes the particular phytocannabinoid for which it is named, as well as derivatives thereof. For example, cannabigerol-type phytocannabinoids includes cannabigerol (CBG) and various derivatives and analogs thereof. Likewise, the other cannabis phytocannabinoids types above will be understood to include at least cannabichromene (CBC), cannabidiol (CBD), tetrahydrocannabinol (THC), cannabinol (CBN), cannabielsoin (CBE), iso-tetrahydrocannabinol (iso-THC), cannabicyclol (CBL), and cannabicitran (CBT), respectively. As such, reference to the classification via the namesake cannabis phytocannabinoid may be used to refer collectively to the members of the class as a whole. For example, it is to be understood that collective terms/phrases such as “a tetrahydrocannabinol” and “the tetrahydrocannabinols” may be used synonymously with terms/phrases like “a tetrahydrocannabinol-type” and “the tetrahydrocannabinol-type phytocannabinoids,” respectively, to refer to tetrahydrocannabinol (THC) and THC derivatives/analogs/etc., which are exemplified and described in further detail below.

Owing to the common structural motifs, the classes above may be considered as derivative of CBG-type phytocannabinoids. Specifically, the chemical classification is broadly based on the derivations from the common C21 precursor cannabigerolic acid (CBGA), or the C19 analog thereof, cannabigerovaric acid (CBGVA), which are the predominate phytocannabinoid precursors and formed via reaction of geranyl pyrophosphate with olivetolic and divarinic acid, respectively. As will be understood by those of skill in the art, most of the cannabis phytocannabinoids are prepared biosynthetically as carboxylic acids (e.g. Δ9-THCA, CBDA, CBCA, etc.), which are subsequently decarboxylated to form the corresponding neutral compounds (i.e., Δ9-THC, CBD, CBC, etc.). Other cannabis phytocannabinoids may be prepared by non-biosynthetic routes, or at the least via routes comprising non-biosynthetic reactions, which are exemplified by the formation of CBN via oxidative degradation of Δ9-THC. As such, with respect to the various cannabis phytocannabinoids described and referenced herein, it is to be appreciated that terms such as “analog,” “derivative,” “modification,” etc. are used to encompass phytocannabinoids prepared/formed as reaction products (i.e., child compounds) of other characterized phytocannabinoids (i.e., parent compounds). Such reaction product/child compound phytocannabinoids may not be isolable, or may not have been previously isolated and/or characterized, but will nonetheless be understood to fall within the scope of derivatives of the parent compound. Isomers and tautomers will also be understood to be included in the scope of the term analog as used herein.

As introduced above, the particular phytocannabinoid profile of the cannabis extract may be selected by the particular cannabis material chosen for the extraction, the particular extraction process utilized, and any pre- and/or post-extraction processing performed in the preparation of the cannabis extract. Moreover, while phytocannabinoids are generally abundant in the resin-producing trichomes as described above, different cultivars of Cannabis plants are known to produce varying amounts of different phytocannabinoids may thus be characterized into chemotypes based on the phytocannabinoid profile of products prepared therefrom. For example, it will be understood by those of skill in the art that the chemotype recognized as producing a minimal amount of (THC) and relatively high levels of cannabidiol (CBD) can be classified as “industrial hemp,” as compared to the “marijuana” chemotype containing higher levels of THC, as introduced with respect to the compliant products described above. Even with such hemp chemotypes however, the flowering tops and leaves have significant levels of CBD whereas the stems/stalks and seeds have little to none, such that different selections of the cannabis material for extraction can result in drastically different product profiles. For example, ethanolic and supercritical CO₂ extracts of whole-hemp plants, or flowering tops and leaves, can be used to prepare the cannabis extract as a full-spectrum hemp extract, a broad-spectrum hemp extract, a hemp oil, a phytocannabinoid-rich hemp oil (or extract), etc. Quantitative analysis of a plant's cannabinoid profile is often determined by gas chromatography (GC), or more reliably by gas chromatography combined with mass spectrometry (GC/MS). Liquid chromatography (LC) techniques may also be utilized and, unlike GC methods, can differentiate between the acid and neutral forms of a given phytocannabinoid.

In some embodiments, the cannabis extract comprises at least one, alternatively at least two, alternatively at least three, alternatively at least four, alternatively at least five, alternatively at least six, alternatively at least seven, alternatively at least eight, alternatively at least nine, alternatively at least ten, alternatively at least eleven, alternatively at least twelve different cannabinoids, which may be selected from any of those described herein.

### CBD

In some embodiments, the cannabis extract comprises a cannabidiol. Examples of cannabidiols include cannabidiol (CBD), cannabidiol monomethylether (CBDM), cannabidiolic acid (CBDA), cannabidiorcol (CBD-C1), cannabidivarin (CBDV), and cannabidivarinic acid (CBDVA). Some such examples are shown by following structural formula:

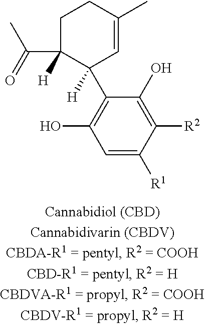

CBD is a major phytocannabinoid constituent of Cannabis plants, and has been demonstrated to possess varied activity against a range of biological targets to provide antidepressant, anxiolytic, and/or neuroprotective effects. For example, in addition to extensive studies concerning CBD for treatment of pain-related conditions, CBD has been shown to display sedative effects, increase alertness, relieve convulsion, reduce inflammation, reduce anxiety, relieve nausea, inhibit growth and invasiveness of particular aggressive cancer cells (e.g. via inhibition of Id-1 gene expression), decrease activity of the limbic system, and treat dystonia, among other beneficial effects, with but very limited side effects (e.g. tiredness, fatigue, decreased appetite). Moreover, CBD has been indicated in reducing adverse effects of THC, e.g. attenuating certain memory-impairing effects of THC and decreasing THC-induced social isolation. CBD has also been demonstrated to decrease the rate of THC clearance from the body, e.g. via interfering with liver-mediated THC metabolism, leading to increased plasma concentrations and thus availability of THC to receptors and ultimately a dose-dependent enhancement of THC effects.

With regard to the human endocannabinoid system, CBD has low affinity for CB1 and CB2 receptors, but nonetheless acts as an indirect antagonist of CB1 and CB2 agonists. With regard to other systems, CBD is believed to be an antagonist of G protein-coupled receptor GPR55, an inverse agonist of GPR3, GPR6, and GPR, a partial agonist of serotonin 5-HT1A receptor, and an allosteric modulator of the μ- and δ-opioid receptors, as well as an inducer of PPARγ agonism and an inhibitor of voltage-gated cation channels and intracellular calcium release. In the context of the present embodiments, it is believed that CBD may be exploited in novel ways against these, and other biological targets described herein, to provide beneficial and/or therapeutic effects to subjects having related conditions. However, selective and general targeting of these biological targets present numerous challenges, including many associated with conventional delivery methods (e.g. due to CBD possessing a high partition coefficient (log P˜6.3). For example, while in vitro diffusion studies have indicated that CBD may permeate human tissue, it is believed that topical absorptive capability in humans has been identified prior to the discoveries and embodiments herein, which will be understood in further detail in view of the description below.

As introduced above, the cannabis extract may comprise cannabidiols other than CBD. For example, in certain embodiments, the cannabis extract comprises at least one cannabidiol selected from cannabidiol monomethylether (CBDM), cannabidiolic acid (CBDA), cannabidiorcol (CBD-C1), cannabidivarin (CBDV), cannabidivarinic acid (CBDVA), and the like, as well as derivatives, modifications, and combinations thereof. These cannabidiols may exhibit the same or different type of bioactivity as those of CBD described above. For example, cannabidivarinic acid (CBDVA) is believed to have anticonvulsant effects, and may be utilized in the treatment of epilepsy of related conditions.

Notwithstanding the above, in some embodiments, the cannabis extract is free from, alternatively substantially free from, such a cannabidiol. For example, in some such embodiments, the cannabis extract comprises a CBD content of from 0 to 5, alternatively from 0 to 3, alternatively from 0 to 1, alternatively from 0 to 0.5, alternatively from 0 to 0.3, alternatively from 0 to less than 0.3, alternatively from 0 to 0.1, alternatively from 0 to 0.05, alternatively from 0 to 0.01 wt. %, based on the total weight of the cannabis extract.

### THC

In some embodiments, the cannabis extract comprises a tetrahydrocannabinol. Examples of tetrahydrocannabinols include delta-9-tetrahydrocannabinols and delta-8-tetrahydrocannabinols. For purposes of clarity and consistency, the delta-9-tetrahydrocannabinols may be simply designated with the acronym “THC” herein, while the delta-8-tetrahydrocannabinols will typically use the designation Δ8-THC herein.

Examples of delta-9-tetrahydrocannabinols include delta-9-tetrahydrocannabinol (Δ9-THC, or more simply, THC), delta-9-tetrahydrocannabinol-C4 (THC-C4), delta-9-tetrahydrocannabinolic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorcolic acid (THCA-C1), delta-9-tetrahydrocannabivarin (THCV), delta-9-tetrahydrocannabivarinic acid (THCVA), etc.). Some such examples of delta-9-tetrahydrocannabinols are shown by following structural formula:

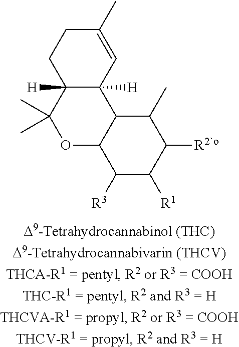

Examples of delta-8-tetrahydrocannabinols include delta-8-tetrahydrocannabinol (Δ8-THC) and delta-8-tetrahydrocannabinolic acid (Δ8-THCA).

THC is the principal psychoactive constituent (i.e., phytocannabinoid) of Cannabis plants, with activity resulting in alteration to a subject's visual, auditory, and olfactory senses. As introduced above, the initially synthesized and accumulated form of THC in such plants is THCA. THC has mild to moderate analgesic effects, and thus can be used to treat pain. For example, THC can induce alteration of transmitter release on dorsal root ganglion of the spinal cord, and in the periaqueductal gray (i.e., the central gray). THC also has marked antiemetic activity, and may induce relaxation and/or reduce aggression in certain subjects. THC may also induce fatigue and stimulate appetite.

The pharmacological actions of THC result from its partial agonist activity at the cannabinoid receptor CB1, located mainly in the central nervous system, and the CB2 receptor, mainly expressed in cells of the immune system. The psychoactive effects of THC are primarily mediated by activation of CB1G-protein coupled receptors, leading to a decrease in concentrations of the second messenger molecule cAMP through inhibition of adenylate cyclase. Recently, THC has been shown to possess anticholinesterase activity.

In some embodiments, the cannabis extract comprises a delta-9-tetrahydrocannabinol, selected from delta-9-tetrahydrocannabinol (THC), delta-9-tetrahydrocannabinol-C4 (THC-C4), delta-9-tetrahydrocannabinolic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorcolic acid (THCA-C1), delta-9-tetrahydrocannabivarin (THCV), delta-9-tetrahydrocannabivarinic acid (THCVA), and the like, as well as derivatives, modifications, and combinations thereof.

Δ8-THC is another psychoactive constituent of Cannabis plant, and has been shown to be neuroprotective and demonstrate analgesic, antiemetic, anxiolytic, and orexigenic properties. In some embodiments, the cannabis extract comprises a delta-8-tetrahydrocannabinol selected from delta-8-tetrahydrocannabinol (Δ8-THC), delta-8-tetrahydrocannabinolic acid (Δ8-THCA), and the like, as well as derivatives, modifications, and combinations thereof.

In some embodiments, the cannabis extract is free from, alternatively substantially free from delta-9-tetrahydrocannabinols. In these or other embodiments, the cannabis extract is free from, alternatively substantially free from delta-8-tetrahydrocannabinols. In specific embodiments, the cannabis extract is free from, alternatively substantially free from tetrahydrocannabinols. In certain embodiments, the cannabis extract comprises a THC content of from 0 to 5, alternatively from 0 to 3, alternatively from 0 to 1, alternatively from 0 to 0.5, alternatively from 0 to 0.3, alternatively from 0 to less than 0.3, alternatively from 0 to 0.1, alternatively from 0 to 0.05, alternatively from 0 to 0.01 wt. %, based on the total weight of the cannabis extract.

In addition to, or as an alternative of CBD and/or THC, the cannabis extract may comprise other phytocannabinoids, such as, CBG, CBN, CBC, CBL, CBT, CBEA, THCV, CBDA, CBDV, and the like, or combinations thereof. Although these phytocannabinoids are typically found in lower levels in cannabis materials than CBD and/or THC, it will be appreciated that the cannabis extract may be prepared to provide a beneficial amount of phytocannabinoids other than CBD and THC to the cannabis composition.

### CBG

In some embodiments, the cannabis extract comprises a cannabigerol. Examples of cannabigerols include cannabigerol (CBG), cannabigerol monomethylether (CBGM), cannabigerolic acid (CBGA), cannabigerolic acid monomethylether (CBGAM), cannabigerovarin (CBGV), and cannabigerovarinic acid (CBGVA). Some such examples are shown by following structural formula:

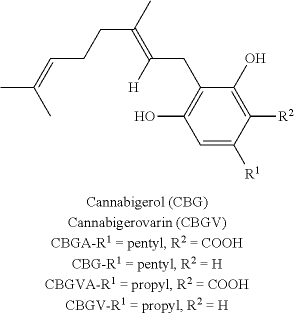

CBG is a non-psychoactive cannabinoid found in higher concentrations in hemp rather than in varieties of Cannabis cultivated for high THC content and their corresponding psychoactive properties. Cannabigerol has been found to act as a high affinity a2-adrenergic receptor agonist, moderate affinity 5-HT1A receptor antagonist, and low affinity CB1 receptor antagonist. It also binds to the CB2 receptor. Cannabigerol has been shown to relieve intraocular pressure, which may be of benefit in the treatment of glaucoma. It is believed that CBG has potential for positively affecting mood benefits via GABA uptake inhibition, and skin benefits via inhibition of keratinocyte proliferation.

In some embodiments, the cannabis extract comprises a cannabigerol, such as cannabigerol (CBG), cannabigerol monomethylether (CBGM), cannabigerolic acid (CBGA), cannabigerolic acid monomethylether (CBGAM), cannabigerovarin (CBGV), cannabigerovarinic acid (CBGVA), and the like, as well as derivatives, modifications, and combinations thereof. In other embodiments, however, the cannabis extract is free from, alternatively substantially free from, such a cannabigerol.

### CBN

In some embodiments, the cannabis extract comprises a cannabinol or cannabinodiol. Examples of cannabinols and cannabinodiols include cannabinodiol (CBND), cannabinodivarin (CBVD), cannabinol (CBN), Cannabinol methylether (CBNM), cannabinol-C2 (CBN-C2), cannabinol-C4 (CBN-C4), cannabinolic acid (CBNA), cannabiorcool (CBN-C1), and cannabivarin (CBV). Some such examples are shown by the following structural formulae:

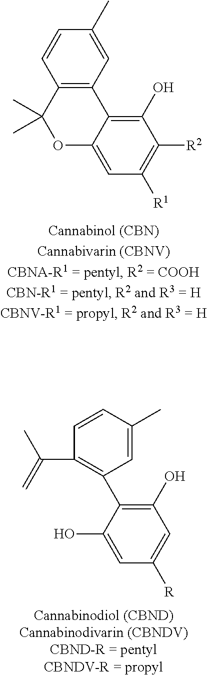

CBN is a psychoactive substance cannabinoid found in Cannabis sativa and Cannabis indica/afghanica. It is also a metabolite of tetrahydrocannabinol (THC). CBN acts as a weak agonist of the CB1 and CB2 receptors, with lower affinity in comparison to THC.

Cannabivarin, also known as cannabivarol or CBV, is a non-psychoactive cannabinoid found in minor amounts in the hemp plant Cannabis sativa. It is an analog of cannabinol (CBN) with the side chain shortened by two methylene bridges (—CH2-). CBV is an oxidation product of tetrahydrocannabivarin (THCV, THV).

In some embodiments, the cannabis extract comprises a cannabinol or cannabinodiol, such as cannabinodiol (CBND), cannabinodivarin (CBVD), cannabinol (CBN), Cannabinol methylether (CBNM), cannabinol-C2 (CBN-C2), cannabinol-C4 (CBN-C4), cannabinolic acid (CBNA), cannabiorcool (CBN-C1), cannabivarin (CBV), and the like, as well as derivatives, modifications, and combinations thereof. In other embodiments, however, the cannabis extract is free from, alternatively substantially free from, such a cannabinol or cannabinodiol.

### CBC

In some embodiments, the cannabis extract comprises a cannabichromene. Examples of cannabichromenes include cannabichromene (CBC), cannabichromenic acid (CBCA), cannabichromevarin (CBCV), and cannabichromevarinic acid (CBCVA). Some such examples are shown by the following structural formula:

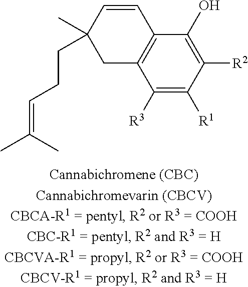

CBC bears structural similarity to the other natural cannabinoids, including tetrahydrocannabinol, tetrahydrocannabivarin, cannabidiol, and cannabinol, among others. Evidence has suggested that it may play a role in the anti-inflammatory and anti-viral effects of cannabis, and may contribute to the overall analgesic effects of medical cannabis.

In some embodiments, the cannabis extract comprises a cannabichromene, such as cannabichromene (CBC), cannabichromenic acid (CBCA), cannabichromevarin (CBCV), cannabichromevarinic acid (CBCVA), and the like, as well as derivatives, modifications, and combinations thereof. In other embodiments, however, the cannabis extract is free from, alternatively substantially free from, such a cannabichromene.

### CBL

In some embodiments, the cannabis extract comprises a cannabicyclol. Examples of cannabicyclols include cannabicyclol (CBL), cannabicyclolic acid (CBLA), cannabicyclovarin (CBLV), and the like, as well as derivatives, modifications, and combinations thereof. Some such examples are shown by the following structural formula:

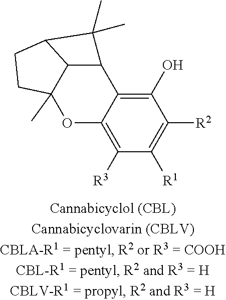

Cannabicyclol (CBL) is a non-psychotomimetic cannabinoid found in the Cannabis species.

In some embodiments, the cannabis extract comprises a cannabicyclol, such as cannabicyclol (CBL), cannabicyclolic acid (CBLA), cannabicyclovarin (CBLV), and the like, as well as derivatives, modifications, and combinations thereof. In other embodiments, however, the cannabis extract is free from, alternatively substantially free from, such a cannabicyclol.

### CBT

In some embodiments, the cannabis extract comprises a cannabitriol. Examples of cannabitriols include 10-ethoxy-9-hydroxy-delta-6a-tetrahydrocannabinol, 8,9-dihydroxy-delta-6a-tetrahydrocannabinol, cannabitriol (CBT), and cannabitriolvarin (CBTV). Some such examples are shown by the following structural formula:

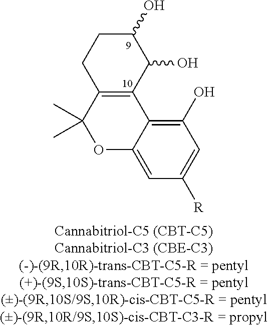

In some embodiments, the cannabis extract comprises a cannabitriol, such as 10-ethoxy-9-hydroxy-delta-6a-tetrahydrocannabinol, 8,9-dihydroxy-delta-6a-tetrahydrocannabinol, cannabitriol (CBT), cannabitriolvarin (CBTV), and the like, as well as derivatives, modifications, and combinations thereof. In other embodiments, however, the cannabis extract is free from, alternatively substantially free from, such a cannabitriol.

### CBEA

In some embodiments, the cannabis extract comprises a cannabielsoin. Examples of cannabielsoins include cannabielsoic acid B (CBEA-B), cannabielsoin (CBE), and cannabielsoin acid A (CBEA-A). Some such examples are shown by the following structural formula:

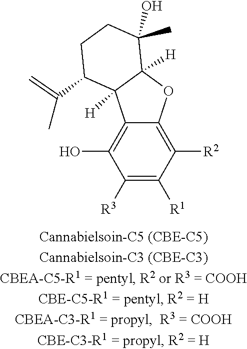

In some embodiments, the cannabis extract comprises a cannabielsoin, such as cannabielsoic acid B (CBEA-B), cannabielsoin (CBE), cannabielsoin acid A (CBEA-A), and the like, as well as derivatives, modifications, and combinations thereof. In other embodiments, however, the cannabis extract is free from, alternatively substantially free from, such a cannabielsoin.

### THCV/THV

In some embodiments, the cannabis extract comprises tetrahydrocannabivarin (THCV, or THV). THCV is a naturally-occurring terpeno-phenolic homolog of THC, having a propyl (3-carbon) side chain. THCV has been shown to be a CB1 receptor antagonist and a cannabinoid receptor type 2 partial agonist, and has recently been implicated in positive treatments of obesity-associated glucose intolerance and type 2 diabetes.

In some embodiments, the cannabis extract comprises THCV or a derivative or modification thereof. In other embodiments, however, the cannabis extract is free from, alternatively substantially free from, such a tetrahydrocannabivarin.

### Other Cannabis Phytocannabinoids

In some embodiments, the cannabis extract comprises a phytocannabinoid not grouped and/or classified with the phytocannabinoids set forth above, such as 10-oxo-delta-6a-tetrahydrocannabinol (OTHC), cannabichromanon (CBCF), cannabifuran (CBF), cannabiglendol, cannabiripsol (CBR), cannabicitran (CBT), dehydrocannabifuran (DCBF), delta-9-cis-tetrahydrocannabinol (cis-THC), tryhydroxy-delta-9-tetrahydrocannabinol (triOH-THC), 3,4,5,6-tetrahydro-7-hydroxy-alpha-alpha-2-trimethyl-9-n-propyl-2,6-methano-2H-1-benzoxocin-5-methanol (OH-iso-HHCV), and the like, as well as derivatives, modifications, and combinations thereof. In other embodiments, however, the cannabis extract is free from, alternatively substantially free from, such a miscellaneous phytocannabinoid (i.e., a phytocannabinoid not grouped and/or classified with the particular cannabis phytocannabinoids described herein).

In certain embodiments, the cannabis extract comprises at least two, alternatively at least three, alternatively at least four, alternatively at least five, alternatively at least six, alternatively at least seven, alternatively at least eight, alternatively at least nine, alternatively at least ten, alternatively at least eleven different phytocannabinoids. In some such embodiments, at least one of the phytocannabinoids is a cannabidiol-type phytocannabinoid, such as cannabidiol (CBD). In certain embodiments, the cannabis extract comprises at least one phytocannabinoid selected from cannabinol (CBN), tetrahydrocannabinolic acid (THCA), cannabidiolic acid (CBDA), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), cannabicitran (CBT). In other embodiments, the cannabis extract comprises cannabidiol (CBD) and at least one phytocannabinoid selected from cannabidiolic acid (CBDA), cannabidivarin (CBDV), cannabidivarinic acid (CBDVA), cannabigerol (CBG), cannabigerolic acid (CBGA), cannabinol (CBN), and cannabichromene (CBC).

In specific embodiments, the cannabis extract can be enriched in one or more phytocannabinoids, i.e., comprises a higher proportion of a particular phytocannabinoid over one other phytocannabinoid, alternatively over a combination of other phytocannabinoids, in the cannabis extract, as compared against the proportions of phytocannabinoids in the cannabis material that was extracted. For example, in certain embodiments, the cannabis extract is enriched in a cannabidiol-type phytocannabinoid, such as cannabidiol (CBD). In particular such embodiments, the cannabis extract comprises a therapeutically effective amount of the cannabidiol-type phytocannabinoid.

In some embodiments, the cannabis extract is enriched in a cannabigerol-type phytocannabinoid, such as cannabigerol (CBG), cannabigerolic acid (CBGA), and/or cannabigerovarin (CBGV). In particular such embodiments, the cannabis extract comprises a therapeutically effective amount of the cannabigerol-type phytocannabinoid.

In some embodiments, the cannabis extract is enriched in a cannabinol-type phytocannabinoid, such as cannabinol (CBN), cannabidiolic acid (CBDA), cannabidivarin (CBDV), and/or cannabidivarinic acid (CBDVA). In particular, such embodiments, the cannabis extract comprises a therapeutically effective amount of the cannabinol-type phytocannabinoid.

In some embodiments, the cannabis extract is enriched in a cannabichromene-type phytocannabinoid, such as cannabichromene (CBC). In particular such embodiments, the cannabis extract comprises a therapeutically effective amount of the cannabichromene-type phytocannabinoid.

In other embodiments, the cannabis extract is free from, alternatively substantially free from, one or more of the phytocannabinoids set forth herein. For example, in certain embodiments, the cannabis extract is free from, alternatively substantially free from tetrahydrocannabinol (THC) and related tetrahydrocannabinol-type phytocannabinoids. In some of these embodiments, the cannabis extract is also free from, alternatively substantially free from at least one phytocannabinoid selected from cannabinol (CBN), tetrahydrocannabinolic acid (THCA), cannabidiolic acid (CBDA), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabielsoin (CBE), and cannabicitran (CBT). In additional embodiments, the cannabis extract is free from tetrahydrocannabinol (THC) and at least two, alternatively at least three, alternatively at least four phytocannabinoids independently selected from the preceding list.

### Amounts

Generally, the one or more cannabinoids may be present in the cannabis extract in any suitable concentrations, which can be selected or otherwise determined based on requirements of specific end applications. In some embodiments, for example, the cannabis extract comprises a cannabinoid fraction (i.e., the content of the combined amount of phytocannabinoids) of at least 1%, alternatively at least 2, alternatively at least 5, alternatively at least 10, alternatively at least 15, alternatively at least 20, alternatively at least 25, alternatively at least 30, alternatively at least 35, alternatively at least 40, alternatively at least 50, alternatively at least 55, alternatively at least 60, alternatively at least 65, alternatively at least 70, alternatively at least 75, alternatively at least 80, alternatively at least 85, alternatively at least 90, alternatively at least 95, alternatively at least 98 wt. %, based on the total weight of the cannabis extract. In some embodiments, the cannabinoid fraction is selected at one of the same % values, but is compared on a w/v or v/v basis against the cannabis extract as a whole.

In some embodiments, the cannabis extract comprises the cannabinoid fraction in an amount of from 1 to 99 wt. %, such as from greater than 1 to 99, alternatively from 10 to 99, alternatively from 20 to 99 alternatively from 30 to 99, alternatively from 40 to 99, alternatively from 50 to 99, alternatively from 60 to 99, alternatively from 70 to 99, alternatively from 75 to 99, alternatively from 80 to 99 wt. %, based on the total weight of the cannabis extract. Alternatively, the cannabis extract comprises the cannabinoid fraction in an amount of from 1 to less than 99 wt. %, such as from 1 to 95, alternatively from 1 to 90 alternatively from 1 to 80, alternatively from 1 to 75, alternatively from 1 to 70, alternatively from 1 to 60, alternatively from 1 to 50, alternatively from 1 to 40, alternatively from 1 to 25 wt. %, based on the total weight of the cannabis extract.

In certain embodiments, the cannabis extract comprises a single phytocannabinoid in an amount ≥99% (w/w or v/v). In such embodiments, the cannabis extract may be further defined as an isolate, or single compound, in terms of the particular phytocannabinoid therein. For example, in some such embodiments, the cannabis extract is a CBD isolate comprising ≥99 wt. % CBD, based on the total weight of the cannabis extract. In other embodiments, the cannabis extract comprises a single phytocannabinoid in an amount ≥80% (w/w or v/v). In such embodiments, the cannabis extract may be further defined as a distillate of the particular phytocannabinoid therein. For example, in some such embodiments, the cannabis extract is a CBG distillate comprising ≥80 wt. % CBG, based on the total weight of the cannabis extract. In yet other embodiments, the cannabis extract comprises a cannabinoid fraction comprising at least 5, alternatively at least 6, alternatively at least 7, alternatively at least 8 different phytocannabinoids, each in an amount of from 1 to 80 wt. % of the cannabis extract. In such embodiments, the cannabis extract may be further defined as a full spectrum cannabis extract. In some such embodiments, the cannabis extract comprises a total THC content of <0.3%, such that the cannabis extract may be further defined as a compliant full spectrum cannabis extract.

### Other Extractables

As introduced above, in certain embodiments, the cannabis extract may include other phytocompounds aside from, or in place of, the phytocannabinoids described above. In certain embodiments, the cannabis extract comprises one or more terpenes, terpenoids, flavonoids, phenolics, glycosides, alkaloids, and the like, as well as derivatives thereof, as will be appreciated in view of the further description and examples herein.

Isoprenes: Terpenes and Terpenoids (e.g. Sesquiterpenes)

In certain embodiments, the cannabis extract comprises one or more terpene compounds. As used herein, the term “terpene compound” is used to refer to a naturally-occurring terpene or terpenoid, or a derivative thereof. As will be understood by those of skill in the art, such terpene compounds compose a very large and diverse class of organic phytocompounds produced by a variety of plants, including Cannabis plants.

Terpene compounds are typically classified in terms of a characteristic polyisoprene backbone, which is formed via isoprenoid precursor biosynthesis involving the mevalonate pathway (i.e., the HMG-CoA reductase pathway, isoprenoid pathway, etc.,), which takes place in the cytosol and/or the non-mevalonate pathway (i.e., the mevalonate-independent pathway, 2-C-methyl-D-erythritol 4-phosphate/1-deoxy-D-xylulose 5-phosphate (MEP/DOXP) pathway, etc.), which takes place in plastids. As the name implies, the polyisoprene backbone is conventionally considered in terms of individual units derived from isoprene (i.e., H₂CC(CH₃)CHCH₂). In this fashion, terpene compounds, which are also called “isoprenes” or “isoprenoids” depending on the absence or presence of non-hydrocarbon functional groups, respectively, may be described as polymers of isoprene via unit formula (C₅H₈)ₙ, where n is the number of isoprene subunits linked together in the polyisoprene backbone. These isoprene subunits are typically linked “head to tail” in linear chains, rings, etc., and may be used in the naming of a particular terpene compound. For example, hemiterpenes, monoterpenes, sesquiterpenes, diterpenes, sesterterpenes, triterpenes, sesquarterpenes, and tetraterpenes represent terpenes with 1, 2, 3, 4, 5, 6, 7, and 8 isoprene subunits, respectively. In this same fashion, hemiterpenoids represent terpenoids having but one isoprene unit (i.e., 5 carbons), monoterpenoids represent terpenoids having two isoprene units (i.e., 10 carbons), and sesquiterpenoids, diterpenoids, sesterterpenoids, triterpenoids, sesquarterpenoids, tetraterpenoids, and polyterpenoids with represent terpenoids with 3, 4, 5, 6, 7, 8, and larger isoprene subunits, respectively.

Examples of hemiterpenes and hemiterpenoids include 2-methyl-1,3-butadiene, hemialboside, hymenoside, and the like, as well as derivatives, modifications, and combinations thereof. Examples of monoterpenes and hemiterpenoids include borneol, camphene, camphor, Δ-3-carene, carvone eucalyptol, fenchol, geraniol, hinokitiol, isoborneol, limonene, linalool, myrcene, menthol, nerol, α-ocimene; β-ocimene, cis-ocimene, ocimene, pinene, α-pinene, β-pinene, cis-pinane, trans-pinane, cis-pinanol, trans-pinanol, α-phellandrene, β-phellandrene, trans-2-pinanol, sabinene, α-terpinene, terpinolene, α-terpinolene, β-terpinolene, γ-terpinolene, Δ-terpinolene, α-terpineol, and the like, as well as derivatives, modifications, and combinations thereof. Examples of sesquiterpenes and sesquiterpenoids include α-bisabolene, β-bisabolene, bisabolol, caryophyllene, caryophyllene oxide, α-cedrene, β-cedrene, elemene, α-elemene, β-elemene, γ-elemene, Δ-elemene, β-eudesmol, eudesm-7(11)-en-4-ol, farnesene, α-farnesene, 0-farnesene, farnesol, guaiol, α-guaiene, β-guaiene, Δ-guaiene, guaiene, germacrene, germacrene A, germacrene B, germacrene C, germacrene D, germacrene E, humulene, α-humulene, nerolidol, santalol, selinene, selina-3,7(11)-diene, valencene, and the like, as well as derivatives, modifications, and combinations thereof. Examples of diterpenes and diterpenoids include oridonin, cafestol, kahweol, cembrene, taxadiene, retinol, retinal, phytol, isophytol, and the like, as well as derivatives, modifications, and combinations thereof. Examples of triterpenes and triterpenoids include ursolic acid, oleanolic acid, and the like, as well as derivatives, modifications, and combinations thereof. Examples of sesquarterpenes and sesquarterpenoids include ferrugicadiol, tetraprenylcurcumene, and the like, as well as derivatives, modifications, and combinations thereof. Examples of tetraterpenes and tetraterpenoids include lycopene, alpha-carotene, beta-carotene, gamma-carotene, and the like, as well as derivatives, modifications, and combinations thereof.

Notwithstanding the above, however, it is understood that the molecule isoprene is not polymerized in terpene bioproduction, but instead that the isomers dimethylallyl pyrophosphate (DMAPP) and isopentenyl pyrophosphate (IPP) are the actual biosynthetic precursors to terpenic isoprene units. DMAPP and IPP are employed, both independently and collectively, in a wide variety of complex biosynthetic metabolic pathways, resulting in the diversity of the terpene compounds as a class. For example, geranyl pyrophosphate (GPP), which is used by Cannabis plants to produce cannabinoids, is formed via geranyl pyrophosphate (GPP) synthase-mediated condensation of DMAPP and IPP. GPP can be converted into monoterpenoids by limonene synthase. Similarly, DMAPP and IPP are ligated by FPP synthase to produce farnesyl pyrophosphate (FPP), which can be used to produce sesquiterpenoids.

It will be understood that terpenes and terpenoids are generally distinguished by the absence or presence of non-hydrocarbon functional groups, respectively, with terpenes comprising C5x hydrocarbons and terpenoids typically comprising oxygen-containing functionality (e.g. alcohol, carbonyl, etc.) or other chemical modifications. For example, in some instances, some natural terpenes may not be readily isolable via extraction, e.g. due to oxidative denaturation that occurs during pre-extraction drying and/or curing stages, whereas the oxidized terpenoid analog thereof is more readily isolated via the same extraction.

Terpene compounds function as essential building blocks of complex plant hormones and molecules, pigments, sterols, and even cannabinoids (i.e., in Cannabis plants). Concerning the latter, it is believed that over 140 unique terpene compounds have been extracted from Cannabis plants in particular. These cannabis terpene compounds are typically synthesized in secretory cells inside glandular trichomes, and may be found in high concentrations in unfertilized female cannabis flowers prior to senescence. Cannabis terpene compounds are typically produced at rates/in elevated amounts in response to increased light exposure. As such, it will be appreciated that various factors, such as Cannabis plant age, maturation, time of day, climate, and even weather can affect the amount and ratios of terpene compounds in a given cannabis material, and likewise an extract obtained therefrom.

Terpene compounds play an important role in providing plants with protection from bacteria, fungus, insects, and other environmental stresses, e.g. via direct chemical action (e.g. antibacterial, cytotoxic, irritant activity) or indirectly via olfaction. For example, the fragrance of fruits and flowers is primarily due to aerosolized terpenes that are registered by the olfactory receptor neurons in the nose. In citrus fruits, the major aromatic components include the terpene compounds limonene and eucalyptol whereas the aromatic components of clove oil include the terpene compounds eugenol and β-caryophyllene and the aromatic components of peppermint include the terpenes compounds menthone, menthol, and limonene. In this same fashion cannabis terpene compounds are responsible for the aromas of Cannabis plants, which vary greatly from one source to another, and even from one harvest to another. Cannabis plants with relatively (or, at least comparatively) high concentrations of specific terpene compounds may even be identified by smell over other strains. Moreover, terpene compounds may be used to enhance or add to the function of some cannabis products. For example, some varieties of Cannabis plants, or materials therefrom, that smell of musk or clove (e.g. due to high levels of the terpene myrcene) may be used to promote sedative and/or relaxing effects, whereas those with piney smells (e.g. from the terpene pinene) may be used to promote mental alertness and memory retention, and those with lemon-like aromas (e.g. from the terpene limonene) may be used to promote mood and/or attitude enhancement.

Terpene compounds are recognized for their diverse biological activity, and have been increasingly investigated for antibacterial, antineoplastic, and other exploitable properties, some of which have been used in traditional herbal remedies. Importantly, some terpene compounds are believed to synergize with certain endogenous and exogenous ligands, i.e., to provide an enhanced or altogether different biological effect than exhibited solely by such ligands. In some such instances, the terpene compounds may be recognized as “entourage compounds,” referring to a terpene compound (or group of terpene compounds) capable of increasing the effects of a ligand-receptor interaction, which the terpene compound(s) exhibit little to no affinity for the receptor.

It is believed that some terpene compounds produced by cannabis plants (i.e., cannabis terpene compounds) display unique therapeutic effects, which may contribute to demonstrable entourage effects of cannabis-based medicinal extracts. For example, some phytocannabinoid-terpenoid interactions have been observed to produce synergy with respect to treatment of pain, inflammation, depression, anxiety, addiction, epilepsy, cancer, and fungal and bacterial infections (including methicillin-resistant Staphylococcus aureus, i.e., MRSA). With regard to specific activity, it is believed that some cannabis terpene compounds interact with neurological receptors or otherwise act as neurotransmitters. In particular, it has been observed that some cannabis terpene compounds affect serotonin and dopamine chemistry. Moreover, it is believed that some terpene compounds bind weakly to cannabinoid receptors (e.g. CB1 and/or CB2). Cannabis terpene compounds exhibit other activity as well, including some associated with circulatory and muscular effects. Additionally, certain terpenes can alter the permeability of cell membranes, resulting in increased or decreased transport of certain phytocannabinoids (e.g. THC, CBD, etc.) into a cell.

Notwithstanding the examples above, examples of suitable terpene compounds (i.e., terpenes, terpenoids, derivatives thereof, etc.) for use in the cannabis extract include acetanisole, acetic acid, acetyl cedrene, anethole, anisole, benzaldehyde, bergamotene (e.g. a-cis-bergamotene, a-trans-bergamotene), bisabolol (e.g. β-bisabolol), borneol, bornyl acetate, butanoic/butyric acid, cadinene (e.g. a-cadinene, γ-cadinene), cafestol, caffeic acid, camphene, camphor, capsaicin, carene (e.g. δ-3-carene), carotene, carvacrol, carvone, dextro-carvone, laevo-carvone, caryophyllene (e.g. δ-caryophyllene), caryophyllene oxide, castoreum absolute, cedrene (e.g. a-cedrene, β-cedrene), cedrene epoxide (e.g. a-cedrene epoxide), cedrol, cembrene, chlorogenic acid, cinnamaldehyde (e.g. a-amyl-cinnamaldehyde, a-hexyl-cinnamaldehyde), cinnamic acid, cinnamyl alcohol, citronellal, citronellol, cryptone, curcumene (e.g. a-curcumene, γ-curcumene), p-cymene, decanal, dehydrovomifoliol, diallyl disulfide, dihydroactinidiolide, 7,8-dihydroionone, dimethyl disulfide, eicosane/Icosane, elemene (e.g. β-elemene), estragole, ethyl acetate, ethyl cinnamate, ethyl maltol, eucalyptol/1,8-cineole, eudesmol (e.g. a-eudesmol, β-eudesmol, γ-eudesmol), eugenol, euphol, farnesene, farnesol, fenchol (e.g. β-fenchol), fenchone, geraniol, geranyl acetate, germacrenes, germacrene b, guaia-1(10),11-diene, guaiacol, guaiene (e.g. a-guaiene), guaiol, gurjunene (e.g. a-gurjunene), herniarin, hexanaldehyde, hexanoic acid, humulene (e.g. a-humulene, β-humulene), Ionol (e.g. 3-oxo-a-ionol, β-Ionol), Ionone (e.g. a-Ionone, β-Ionone), ipsdienol, isoamyl acetate, isoamyl alcohol, isoamyl formate, isoborneol, isomyrcenol, isopulegol, isovaleric acid, isoprene, kahweol, lavandulol, limonene, γ-linolenic acid, linalool, longifolene, a-longipinene, lycopene, menthol, methyl butyrate, 3-mercapto-2-methylpentanal, mercaptan/thiols, β-mercaptoethanol, mercaptoacetic acid, allyl mercaptan, benzyl mercaptan, butyl mercaptan, ethyl mercaptan, methyl mercaptan, furfuryl mercaptan, ethylene mercaptan, propyl mercaptan, thenyl mercaptan, methyl salicylate, methylbutenol, methyl-2-methylvalerate, methyl thiobutyrate, myrcene (e.g. β-myrcene), γ-muurolene, nepetalactone, nerol, nerolidol (e.g. trans-nerolidol), neryl acetate, nonanaldehyde, nonanoic acid, ocimene (e.g. β-ocimene), octanal, octanoic acid, p-cymene, pentyl butyrate, phellandrene, phenylacetaldehyde, phenylethanethiol, phenylacetic acid, phytol, pinene, β-pinene, propanethiol, pristimerin, pulegone, quercetin, retinol, rutin, sabinene, sabinene hydrate, cis-sabinene hydrate, trans-sabinene hydrate, safranal, a-selinene, a-sinensal, β-sinensal, β-sitosterol, squalene, taxadiene, terpin hydrate, terpineol, terpine-4-ol, terpinene (e.g. a-terpinene, γ-terpinene), terpinolene, thiophenol, thujone, thymol, a-tocopherol, tonka undecanone, undecanal, valeraldehyde/pentanal, verdoxan, a-ylangene, umbelliferone, vanillin, and the like, as well as derivatives, modifications, and combinations thereof. It will be appreciated that derivatives of terpenes include terpenoids in an analogous form, as well other isoprenoids, and steroids thereof. Likewise, derivatives of the terpene compounds above may be in various forms not specifically listed above, including alpha (i.e., α-, or a-), beta (i.e., β- or b-), gamma (i.e., γ-, or g-), delta (i.e., Δ-, 6-, or d-), and/or oxo-(i.e., oxide) forms, etc., and isomers thereof.

### Cannabis Terpene Compounds

The particular terpene compound profile of the cannabis extract may be selected by the particular cannabis material chosen for the extraction, the particular extraction process utilized, and any pre- and/or post-extraction processing performed in the preparation of the cannabis extract. Moreover, while cannabis terpene compounds are generally abundant in the resin-producing trichomes and unfertilized female cannabis flowers as described above, different cultivars of Cannabis plants may be selected for extraction based on a particular terpene compound production profile, similar to the phytocannabinoid-based cultivar selection described above. The terpene composition of a given sample, such as a Cannabis plant, flower, leaves, etc. can be analyzed with analytical tools, such as chromatography and/or mass spectrometry as described herein (e.g. LC-MS, GC-MS). It will be appreciated that different selections of the cannabis material for extraction, as well as the particular extraction method utilized, can result in drastically different product profiles. For example, the ethanolic and supercritical CO₂ extracts described above may be utilized to prepare the cannabis extract as the hemp oil. Steam distillation and/or vaporization may also be utilized to prepare a hemp oil, which may be further characterized as an essential oil extract. However, it will be appreciated that many cannabis terpene compounds are more volatile and vaporize at lower or around the same temperature as certain phytocannabinoids (e.g. Δ9-THC boils at ˜157° C., and CBD boils between ˜160-180° C., with lower sublimation temperatures). As such, the particular extraction parameters chosen may prepare the cannabis extract as enriched in cannabis terpene compounds over phytocannabinoids (e.g. wt./wt., collectively) enriched in any particular cannabis terpene compound over another terpene compound (e.g. in terms of wt. %), enriched in a cannabis terpene compound over any phytocannabinoid, or vice versa with respect to any of the preceding selections.

In some embodiments, the cannabis extract comprises a cannabis terpene compound selected from the group consisting of cannabis hemiterpenes, hemiterpenoids, monoterpenes, monoterpenoids, sesquiterpenes, sesquiterpenoids, diterpenes, diterpenoids, sesterterpenes, sesterterpenoids, triterpenes, triterpenoids, sesquarterpenes, sesquarterpenoids, tetraterpenes, tetraterpenoids, and polyterpenoids (i.e., terpene compound classes). In specific embodiments, the cannabis extract comprises at least one cannabis terpene compound selected from each of at least two, alternatively at least three, alternatively at least 4 of the preceding terpene compound classes. In these or other embodiments, the cannabis extract is free from, alternatively substantially free from, cannabis terpene compounds belonging to one or more of the terpene compound classes above.

In some embodiments, the cannabis extract comprises at least two, alternatively at least three, alternatively at least four, alternatively at least five, alternatively at least six, alternatively at least seven, alternatively at least eight, alternatively at least nine, alternatively at least ten, alternatively at least eleven, alternatively at least twelve cannabis terpene compounds. In specific embodiments, the cannabis extract comprises 1, 2, 3, 4, 5, 6, 7, 8, 9, or 10 different cannabis terpenes, such as any of those described herein, and/or 1, 2, 3, 4, 5, 6, 7, 8, 9, or 10 cannabis terpenoids, such as any of those described herein. In some embodiments, the cannabis extract comprises one or more cannabis terpenes and is free from, alternatively substantially free from cannabis terpenoids. In other embodiments, the cannabis extract comprises one or more cannabis terpenoids and is free from, alternatively substantially free from cannabis terpenes.

With regard to the cannabis extract, the cannabis terpene compound(s) therein can be purified, analyzed, and/or identified, by any techniques and methods known in the art. Examples of such methodologies and techniques include fractional distillation, flash chromatography, high pressure liquid chromatography (HPLC), the LC-MS and/or GC-MS techniques described herein, as well as other chromatographic and/or spectroscopic techniques including nuclear magnetic resonance (NMR) spectroscopy and matrix-assisted laser desorption/ionization time-of-flight (MALDI-TOF) mass spectrometry (i.e., for analyzing and/or quantifying cannabis terpene compounds, or other phytocompounds, in the cannabis extract).

In some embodiments, the cannabis extract comprises a cannabis terpene compound selected from the group consisting of, bisabolols, borneol, carenes, caryophyllenes, camphenes, citronellols, cymenes, eucalyptols, fenchols, geraniols, terpinenes, guaiols, humulenes, isopulegols, limonenes, linalools, menthols, myrcenes, ocimenes, phellandrenes, phytols, pinenes, terpenolenes, terpinenes, terpineols, valencenes, and combinations thereof. In some such embodiments, the cannabis extract comprises a caryophyllene, humulene, limonene, linalool, myrcene, pinene, or a combination thereof.

In specific embodiments, the cannabis extract comprises β-caryophyllene, α-humulene, d-limonene, linalool, β-myrcene, α-pinene, or a combination thereof.

### β-Caryophyllene

Beta-caryophyllene (BCP) is a natural bicyclic sesquiterpene having the following structure:

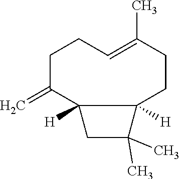

BCP is a constituent of many essential oils, including those of Cannabis sativa, and is one of the predominant sesquiterpenoids isolable from Cannabis plants. BCP is typically isolated as a mixture with isocaryophyllene (i.e., the cis olefin isomer) and α-humulene (i.e., a ring-opened isomer, described further below). BCP is less volatile than many monoterpenoids, and thus may be present in higher concentrations in cannabis extracts or materials processed by heat (e.g. to aid in cannabinoid decarboxylation). In high doses, BCP acts as a calcium and potassium ion channel blocker, and thus may impede pressure exerted by heart muscles (e.g. for antiarrhythmic activity).

With regard to the human endocannabinoid system, BCP is a selective agonist of the CB2 receptor, such that BCP can be further characterized as a phytocannabinoid and, moreover, is the only naturally occurring phytocannabinoid found outside the Cannabis genus. When administered orally, BCP has been shown to possess anti-inflammatory and gastric cytoprotective properties and does not have psychoactive effects. It is believed to exhibit some anti-malarial activity. In topical form, BCP has also been shown to possess anti-inflammatory and analgesic action against toothache.

In the context of the present embodiments, it is believed that BCP may be exploited in novel ways against these and other biological targets described herein, to provide beneficial and/or therapeutic effects to subjects having related conditions. In particular, it is believed that BCP may be utilized in functional compositions (i.e., those incorporating or otherwise prepared from the cannabis extract) for purposes of pain relief, anti-depressant, and/or anti-inflammatory effects.

### α-Humulene

Humulene (i.e., α-humulene, or α-caryophyllene) is a naturally occurring monocyclic sesquiterpene having the following structure:

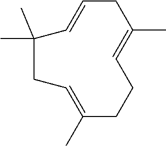

As introduced above, humulene is an isomer of BCP. Like BCP, humulene is believed to exhibit anti-inflammatory properties. Specifically, with regard to the human endocannabinoid system, humulene is believed to induce inhibitory effects on the production of pro-inflammatory cytokines IL-1β and TNFα, and/or prostaglandin PGE2, as well as the expression of enzymes COX-2 and iNOS.

In the context of the present embodiments, it is believed that humulene may be exploited in novel ways against the biological targets above, as well as others described herein, to provide beneficial and/or therapeutic effects to subjects having related conditions. In particular, it is believed that humulene may be utilized in functional compositions (i.e., those incorporating or otherwise prepared from the cannabis extract) for purposes of pain relief (e.g. anti-inflammatory pain) or other inhibition of physiopathologic processes involved with inflammation, and thereby be used to ameliorate a condition such as chronic-degenerative diseases as rheumatoid arthritis, osteoarthritis, systemic lupus erythematosus, ulcerative colitis, psoriasis, atopic eczema, atherosclerosis, and other conditions such as depression, cellulites, and allergies. It is further believed that humulene may be utilized, e.g. in the functional composition, to relieve nausea and/or suppress appetite in a subject.

d-Limonene

D-Limonene is a natural monoterpene having the following structure:

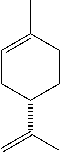

D-Limonene is a major component of aromatic (scents) and resins components of many plants, including Cannabis plants. In additional to utility in aromatherapy, d-limonene is believed to possess antifungal, antibacterial, antioxidant, and anti-inflammatory properties. D-limonene has been shown to affect autonomic nerves, enhance lipolysis via histaminergic response, and reduce appetite and body weight. Additionally, it is believed that d-limonene exhibits anxiolytic-like activity, especially in combination with other phytocompounds such as β-myrcene (described below). Relatedly, d-limonene is believed to synergistically promote absorption of other phytocompounds (e.g. terpenes) by quickly penetrating cell membranes.

In the context of the present embodiments, it is believed that d-limonene may be exploited in novel ways against certain biological targets such as 5-hydroxytryptamine_{1A} (5-HT_{1A}) receptor, as well as others described herein, to provide beneficial and/or therapeutic effects to subjects having related conditions. In particular, it is believed that d-limonene may be utilized in functional compositions (i.e., those incorporating or otherwise prepared from the cannabis extract) for purposes of ameliorating conditions related to nausea and mood disorders.

### Linalool

Linalool is an acyclic monoterpenoid having the following structure:

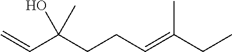

Linalool is a primary component of many plant essential oils implicated in inducing antidepressant-like, anxiolytic-like, and sedative-like activity. Linalool itself has also been shown to possess dose-dependent sedative effects at the central nervous system, including hypnotic, anticonvulsant and hypothermic properties. Moreover, linalool is believed to modulate or inhibit acetylcholine (ACh) release at the neuromuscular junction.

In the context of the present embodiments, it is believed that linalool may be exploited in novel ways against certain biological targets, including those described above as well as other described herein, to provide beneficial and/or therapeutic effects to subjects having related conditions. In particular, it is believed that linalool may be utilized in functional compositions (i.e., those incorporating or otherwise prepared from the cannabis extract) for purposes of ameliorating conditions related to mood disorders (e.g. anxiety), pain, and seizures.

### β-Myrcene

β-Myrcene is a monoterpene having the following structure:

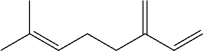

β-myrcene is a major constituent of many essential oils, and is thought to be the predominant terpene found in modern Cannabis cultivars within North America. β-myrcene has been shown to possess antimicrobial, antiseptic, analgesic, antioxidant, anti-carcinogen, anti-depressant, anti-inflammatory, and muscle relaxing effects. Like d-limonene above, β-myrcene is believed to synergistically promote absorption of other phytocompounds via affecting the permeability of the cell membranes. For example, β-myrcene is believed to allow more THC to reach brain cells. Myrcene has also shown cytotoxic effects in crown gall tumors, MCF-7 breast carcinoma, HT-29 colon adenocarcinoma, and P388 leukemia, as well as other cell lines. β-myrcene is believed to possess activity in the central nervous system, as myrcene has elicited an increased barbiturate sleeping time in mice, and mice treated with myrcene have shown decreased spontaneous activity, rearing, and grooming in certain field tests.

In the context of the present embodiments, it is believed that β-myrcene may be exploited in novel ways against certain biological targets, including those described herein, to provide beneficial and/or therapeutic effects to subjects having related conditions. In particular, it is believed that β-myrcene may be utilized in functional compositions (i.e., those incorporating or otherwise prepared from the cannabis extract) for purposes of ameliorating conditions related to muscle pain. Specifically, it is believed that β-myrcene may be exploited to provide such functional compositions with sedative and/or pain-relieving properties.

### α-Pinene

Pinene is a monoterpene having the following structure:

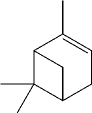

Pinene is found in cannabis plants and, especially, in finished, dried cannabis flowers. Pinene presents in nature as two structural isomers, α-pinene and β-pinene. The enantiomers of α-pinene are shown below:

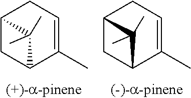

α-Pinene readily crosses the blood-brain barrier and is highly bioavailable, with ˜60% human pulmonary uptake with rapid metabolism or redistribution. α-Pinene exhibits activity as an acetylcholinesterase inhibitor, aiding memory and alleviating/counteracting THC-induced short-term memory troubles and anxiety. (−)-α-Pinene in particular has been shown to be a positive modulator of GABAA receptors, acting at the benzodiazepine binding site. α-Pinene is also believed to act as an expectorant, bronchodilator, and topical antiseptic, and exhibits anti-inflammatory properties via PGE1. α-Pinene has also demonstrated acetylcholinesterase inhibitory activity, which may account for reported effects counteracting THC-induced short-term memory troubles and anxiety. α-Pinene has also been shown to induce euphoria, reduce oil production in oily skin, and possess anti-cancer properties (e.g. via cytostatic activity on tumors).

In the context of the present embodiments, it is believed that α-pinene may be exploited in novel ways against certain biological targets, including those described above, in order to provide beneficial and/or therapeutic effects to subjects having related conditions (e.g. memory trouble, focus problems, etc.). In particular, it is believed that α-pinene may be utilized in functional compositions (i.e., those incorporating or otherwise prepared from the cannabis extract) for purposes of improving focus, short-term memory, and perceived energy/alertness.

### Specific Embodiments of Terpenes

In specific embodiments, the cannabis extract comprises but one of the terpene compounds in the group consisting of β-caryophyllene, α-humulene, d-limonene, linalool, β-myrcene, and α-pinene. In other embodiments, the cannabis extract comprises two, alternatively three, alternatively four, alternatively five, alternatively each of the terpene compounds in the group consisting of β-caryophyllene, α-humulene, d-limonene, linalool, β-myrcene, and α-pinene.

In particular embodiments, the cannabis extract can be enriched in one or more terpene compounds, i.e., comprises a higher proportion of a particular terpene compound over one other terpene compound, alternatively over a combination of other terpene compounds, in the cannabis extract, as compared against the proportions of terpene compounds in the cannabis material that was extracted. For example, in certain embodiments, the cannabis extract is enriched in a β-caryophyllene. In particular such embodiments, the cannabis extract comprises a therapeutically effective amount of β-caryophyllene. In these or other embodiments, the cannabis extract is enriched in α-humulene. In particular such embodiments, the cannabis extract comprises a therapeutically effective amount of the α-humulene. In these or other embodiments, the cannabis extract is enriched in d-limonene. In particular such embodiments, the cannabis extract comprises a therapeutically effective amount of the d-limonene. In these or other embodiments, the cannabis extract is enriched linalool. In particular such embodiments, the cannabis extract comprises a therapeutically effective amount of the linalool. In these or other embodiments, the cannabis extract is enriched in β-myrcene. In particular such embodiments, the cannabis extract comprises a therapeutically effective amount of the β-myrcene. In these or other embodiments, the cannabis extract is enriched in α-pinene. In particular such embodiments, the cannabis extract comprises a therapeutically effective amount of the α-pinene.

In some embodiments, the cannabis extract comprises a cannabis terpene compound profile substantially the same as that of the cannabis material extracted in the preparation of the cannabis extract. In some such embodiments, the ratio between two or more terpene compounds is preserved, such that both the cannabis material and the cannabis extract prepared therewith both comprise the same, alternatively substantially the same ration between the two or more terpene compounds.

Generally, the one or more terpene compounds may be present in the cannabis extract in any suitable concentrations, which can be selected or otherwise determined based on requirements of specific end applications. In some embodiments, for example, the cannabis extract comprises a terpene compound fraction (i.e., the content of the combined amount of terpene compounds therein) of at least 1%, alternatively at least 2, alternatively at least 5, alternatively at least 10, alternatively at least 15, alternatively at least 20, alternatively at least 25, alternatively at least 30, alternatively at least 35, alternatively at least 40, alternatively at least 50, alternatively at least 55, alternatively at least 60, alternatively at least 65, alternatively at least 70, alternatively at least 75, alternatively at least 80, alternatively at least 85, alternatively at least 90, alternatively at least 95, alternatively at least 98 wt. %, based on the total weight of the cannabis extract. In some embodiments, the terpene compound fraction is selected at one of the same % values, but is compared on a w/v or v/v basis against the cannabis extract as a whole.

In some embodiments, the cannabis extract comprises the terpene compound fraction in an amount of from 1 to 99 wt. %, such as from greater than 1 to 99, alternatively from 10 to 99, alternatively from 20 to 99 alternatively from 30 to 99, alternatively from 40 to 99, alternatively from 50 to 99, alternatively from 60 to 99, alternatively from 70 to 99, alternatively from 75 to 99, alternatively from 80 to 99 wt. %, based on the total weight of the cannabis extract. Alternatively, the cannabis extract comprises the terpene compound fraction in an amount of from 1 to less than 99 wt. %, such as from 1 to 95, alternatively from 1 to 90 alternatively from 1 to 80, alternatively from 1 to 75, alternatively from 1 to 70, alternatively from 1 to 60, alternatively from 1 to 50, alternatively from 1 to 40, alternatively from 1 to 25 wt. %, based on the total weight of the cannabis extract.

In some embodiments, the cannabis extract is free from, alternatively substantially free from, one or more of the terpene compounds set forth herein. For example, in certain embodiments, the cannabis extract is free from, alternatively substantially free from 3-caryophyllene, α-humulene, d-limonene, linalool, 3-myrcene, or α-pinene. In some of these embodiments, the cannabis extract is also free from, alternatively substantially free from at least one other terpene compound selected from the same group, i.e., the cannabis extract is at least substantially free from at least two terpene compounds of the group consisting of β-caryophyllene, α-humulene, d-limonene, linalool, β-myrcene, and α-pinene.

It is to be appreciated that, in addition to the embodiments described above, the cannabis extract can be combined or otherwise supplemented with one or more compounds, e.g. for the purposes of enriching the composition of the cannabis extract with respect to those one or more compounds in particular. As such, it is to be appreciated that a terpene compound, such as any of those described above, may be obtained (e.g. via commercial supplier, synthesis, another extraction of the same or different cannabis material, or another material all together, etc.). One of skill in the art will understand that such terpene compounds can be acquired commercially, in various purities, and may be useful biochemical agents for a variety of olfactory and physiologically stimulating purposes, including those described herein.

### Other Phytocompounds

As introduced above, in certain embodiments, the cannabis extract may include other phytocompounds aside from, or in place of, the phytocannabinoids and/or terpene compounds described above. These phytocompounds may be obtained from the cannabis material, i.e., as part of the extraction, or otherwise added to or combined with such an extraction product to prepare the cannabis extract. As such, the other phytocompounds suitable for use in the cannabis extract may be cannabis phytocompounds (i.e., naturally occurring in Cannabis plants, or derivable therefrom), or non-cannabis phytocompounds (i.e., not present or derivable from compounds in Cannabis plants).

Examples of cannabis phytocompounds other than the cannabinoids and terpene compounds described herein include flavonoids, phenolics, glycosides, alkaloids, and the like, as well as derivatives thereof.

### Flavonoids

In some embodiments, the cannabis extract comprises a flavonoid. It will be understood by those of skill in the art that flavonoids are a common constituent of plants, including Cannabis plants, encompass a varied set of compounds with wide ranging functions such as different color pigments (e.g. in petals, leaves, etc.) to attract pollinating insects, receive certain wavelengths of light (e.g. to detect photoperiod), filter harmful ultraviolet light (e.g. to protect the plant), or act as antifungal agents. Flavonoids also exhibit pharmacological activities, including antioxidant, antidiabetic, anti-inflammatory, antiallergic, antibiotic, antidiarrheal, CNS, and anti-cancer. As used herein, the term “flavonoid” is to be understood as encompassing flavonoids, bioflavonoids, isoflavonoids, and neoflavonoids, including the various forms thereof. For example, flavonoids will be understood to encompass anthocyanidins, anthoxanthins, flavones, flavonols, flavanones, flavans, hydroxyflavones, flavan-3-ols, etc.).

In certain embodiments, the cannabis extract comprises a cannabis flavonoid (i.e., a flavonoid extracted from cannabis material, or otherwise derived from a substance so extracted). Examples of cannabis flavonoids include apigenin, chrysoeriol, cosmosiin, flavocannabiside, kaempferol, luteolin, myricetin, orientin, isoorientin (e.g. homoorientin), quercetin, b-sitosterol, (+)-taxifolin, vitexin, isovitexin, and the like, as well as derivatives, modifications, and combinations thereof. As will be understood by those of skill in the art, examples of such derivatives include geraldol, rhamnetin, isorhamnetin, and rhamnazin.

Additional examples of cannabis flavonoids include the cannflavins (e.g. prenylflavonoids) such as cannflavin A, cannflavin B, and cannflavin C. It is believed that the cannflavins possess inhibitory activity against prostalglandins, and therefor may be utilized against inflammation.

In certain embodiments, however, the cannabis extract is free from, alternatively substantially free from, one or more cannabis flavonoids. For example, in some such embodiments, the cannabis extract is free from, alternatively substantially free from, cannflavin A, cannflavin B, and/or cannflavin C.

### Other Phytonutrients

In certain embodiments, the cannabis extract comprises a phytonutrient. As introduced above, such phytonutrients may fall under the definition of cannabis phytocompounds (i.e., naturally occurring in Cannabis plants, or derivable therefrom), or non-cannabis phytocompounds. Additionally, one of skill in the art will understand that some phytonutrients will overlap with some of the phytocompounds introduced above. Such overlap is a function of terms and definitions, as understood by those of skill in the art, and are not to imply limitation to but one particular phytocompound, nor inclusion of multiple phytocompounds, in instances where more than one component can comprise or be such a phytocompound. Rather, the limitations described herein are to be used to understand the scope of the cannabis extract.

Certain general examples of phytonutrients include dietary carbohydrates (e.g. resistant starches), lipids (e.g. omegas 3, 6, etc.), proteins (e.g. whole, isolates, hydrolysates, etc., from soy, whey, rice, pea, etc.), phenylpropanoids (i.e., aromatic compounds comprising a phenylpropane moiety), pteridines, benzopyrans, benzenoids, lignans, neolignans, and the like, as well as derivatives, modifications, and combinations thereof. For example, such phytonutrients may comprise proteins, peptides, complex amino acids (e.g. those found in plant-based protein isolates from soy, rice, wheat, pulses, peas, potatoes, fruit, buckwheat, corn, etc.), branch chain amino acids, medium- and short chain fatty acids (e.g. caproic acids, caprylic acids, capric acids, lauric acids, isovaleric acids, valeric acids, isobutyric acids, butyric acids, propionic acids, acetic acid, formic acid, etc.), hydroxyl acids (e.g. lactic acid, etc.), and the like, as well as derivatives, modifications, and combinations thereof.

Some examples of phytonutrients include phenylpropanoic acids, flavonoids (i.e., compounds comprising a 2-phenylchromene moiety), isoflavonoids (i.e., compounds including a 3-phenylchromen-4-one moiety or a moiety derived therefrom), hydroxyisoflavonoids (i.e., hydroxyl-functional isoflavonoids), isoflav-2-enes (i.e., compounds comprising a 3-phenylchromene moiety having a chromenyl C2-C3 alkene), flavanones (i.e., compounds comprising a flavan-3-one moiety, including those comprising a 2-phenyl-3,4-dihydro-2H-1-benzopyran bearing a C3 ketone), isoflavones (i.e., polycyclic compounds comprising a C4-ketonyl 2-isoflavene moiety), flavans (i.e., compounds comprising a 2-phenyl-3,4-dihydro-2H-1-benzopyran moiety), coumarins (i.e., compounds comprising a 1-benzopyran-2-one moiety), isocoumarins (i.e., C1-ketonyl isochromanes), pterins (i.e., polycyclic aromatic compounds comprising a pterin moiety), chromones (i.e., compounds comprising a benzopyran-4-one moiety), phenols (e.g. benzenediols such as catechols), dibenzylbutane lignans, dibenzylbutanediol lignans and the like, as well as derivatives, modifications, and combinations thereof. Specific examples of phytonutrients may include: 2-(4-hydroxyphenyl)propionate; 2,3-dehydroequol; 2,4,6-trihydroxybenzaldehyde; 2,4,6-trihydroxybenzoic acid; 3-(3,4-dihydroxyphenyl)-acetic acid; 3-(3,4-dihydroxyphenyl)propionate; 3-(3-hydroxyphenyl)-propionic acid; 3-(4-hydroxyphenyl)propionate; 3,4-dihydroxybenzoic acid; 3,4-dihydroxybenzyldehyde; 3,4-dihydroxyphenylacetaldehyde; 3,4-dihydroxyphenylacetate; 3,4-dihydroxyphenylenolpyruvate; 3,4-dihydroxyphenylpyruvate; 4-hydroxypenylacetate; 5-(3′,4′-dihydroxyphenyl)-y-valerolactone; 5-(3′,5′-dihydroxyphenyl)-y-valerolactone; 6′-hydroxy-O-desmethylangolensin; acetate; alpha-2′,3,4,4′,6′-hexahydroxydihydrochalcone; alphitonin; butyrate; daidzein; dihydro-daidzein; enterodiol; enterolactone; equol (e.g. (S)-equol); eriodictyol; ethanol; formate; genistein; glucose; hemoeriodictyol; hesperetin; lactate; 0-desmethylangolensin; phenylacetate; phloroglu-cinol; protocatechuic acid; quercetin; sulfurophane; taxifolin; tetrahydrodaidzein; urolithin A (e.g. 3,8-dihydroxybenzo[c]chromen-6-one), and the like, as well as derivatives, modifications, and combinations thereof.

In some embodiments, the cannabis extract comprises one or more cannabis phytonutrients, such as any of those described above isolable or otherwise obtainable from a Cannabis plant or cannabis material. In these or other embodiments, the cannabis extract is free from, alternatively substantially free from, non-cannabis phytonutrients. In some of these or other embodiments, the cannabis extract is free from, alternatively substantially free from, cannabis phytonutrients not falling within the other particular phytocompound classifications described herein.

In some embodiments, the cannabis extract comprises one or more fatty acids, such as any of the medium- and/or short-chain fatty acids introduced above. In some such embodiments, the particular fatty acids are selected to increase or otherwise enhance the bioavailability of one or more chemical substances in the cannabis extract, i.e., when utilized in the functional composition, upon administration to humans, animals, or other biological systems.

For example, fatty acid-enriched formulations can be utilized to maintain synergistic effects of cannabis phytocompounds that would otherwise be lost during extraction of the cannabis material. In some embodiments, the cannabis extract is a fatty acid-enriched formulation comprising one or more medium-chain fatty acid triesters of glycerol (i.e., a medium-chain triglyceride, or MCT) to provide enhanced bioavailability in a human body. In some such embodiments, the cannabis extract is formulated or otherwise prepared to maintain or enhance the synergistic interaction between the cannabis terpene compounds and phytocannabinoids therein (i.e., the “entourage effect”) in the human body upon administration thereto.

### Forms of Cannabis Extract

As introduced above, the form the of the cannabis extract is not particularly limited, but instead will be selected based on the particular extraction technique(s) utilized during preparation, a desired end-use, etc., as will be understood by those of skill in the art. As such, while particular forms of the cannabis extract are described and exemplified herein, it will be appreciated that derivatives, modifications, combinations, or even alternatives to such forms may be utilized.

### Hemp Seed Oil

In some embodiments, the cannabis extract comprises, alternatively consists essentially of, a hemp seed oil. In such embodiments, the cannabis extract is prepared or otherwise obtained via extracting hemp seed. Typically, such embodiments utilize the cannabis extract as a low-cannabinoid substance comprising cannabis fatty acids and terpene compound. More specifically, hemp seeds contain nutritious omega-3 fatty acids, and are typically high in protein, but only contain trace amounts of phytocannabinoids and little to no terpene compounds. For example, in some such embodiments, the cannabis extract comprises a phytocannabinoid content of <10, alternatively <5, alternatively <3, alternatively <1, alternatively <0.5 wt. %, based on the weight of the cannabis extract. In such embodiments, the balance of the cannabis extract is typically fatty acid. For example, in some such embodiments, the cannabis extract comprises ˜7% saturated fatty acids, ˜9% oleic acid (18:1); ˜54% linoleic acid (18:2); and ˜22% linolenic acid (18:3), and optionally, one or more other fatty acids. In such embodiments, it is believed that the free fatty acids may be able to activate biological targets, including the PPAR (alpha, gamma and beta/delta) nuclear receptors.

### Cannabis Flour

In some embodiments, the cannabis extract comprises, alternatively consists essentially of, a cannabis powder (e.g. a hemp powder), i.e., a dry powder or semidry composition of cannabinoid containing powder obtained from cannabis material. Such a cannabis extract may be referred to as a cannabis flour or dehydrate (or hemp flour/hydrate, depending on the selected feedstock cannabis material), and is exemplified by a cannabis raffinate or other type of cannabis/hemp oil (e.g. any of those described herein) mixed with a thickening agent, drying agent, stabilization agent, flow agent, and/or preserving agent. The resulting composition may then be mechanical mixed, optionally under elevated temperature, to form a dry or semidry powder. In some embodiments, the hemp powder is prepared from cannabis leaf, buds, and/or flowers. In these or other embodiments, the cannabis powder is prepared primarily from cannabis leaf. In some embodiments, preparing cannabis hemp powder comprises a dehydration step. In some such embodiments, the cannabis powder may be referred to as a cannabis dehydrate. In specific embodiments, the cannabis powder is further defined as a hemp flour, hemp powder, and/or hemp dehydrate.

As with the other cannabis extracts described herein, the cannabis powder/flour may comprise a varying amount of phytocompounds, such as phytocannabinoids. For example, a relative percentage of phytocannabinoids in the hemp powder may be adjusted by combining a phytocannabinoid with the powdered mixture described above. As such, the cannabis powder may be enriched with a tetrahydrocannabinol (THC, d9-THC, 1-trans-delta9-tetrahydrocannabinol), cannabidiol (CBD), cannabigerol (CBG), etc., as well as combinations thereof. cannabichromene (CBC), cannabicyclol (CBL), cannabinol (CBN), cannabivarin (CBV), delta-9 Tetrahydrocannabinolic acid (THCA, d9-THCA), Tetrahydrocannabivarin (THCV), Cannabidiolic acid (CBDA), Cannabigerolic acid (CBGA), Cannabidivarin (CBDV), Cannabichromevarin (CBCV), Cannabigerovarin (CBGV), and Cannabigerol Monomethyl Ether (CBGM).

The cannabis powder may be utilized in any amount in the cannabis composition. For example, in certain embodiments, the cannabis powder is utilized in an amount of from 1 to 60 wt. %, such as from 2 to 60, alternatively from 5 to 60, alternatively from 10 to 60, alternatively from 15 to 60, alternatively from 20 to 60, alternatively from 20 to 55, alternatively from 20 to 50 wt. %, based on the total weight of the cannabis composition.

### Cannabis Juice

In some embodiments, the cannabis extract comprises, alternatively consists essentially of, a cannabis juice. It will be appreciated that the cannabis juice may be obtained from myriad cannabis materials described herein, e.g. via a juicing process (e.g. cold-pressing). In certain embodiments, the cannabis juice is prepared from cannabis obtained from freshly harvested cannabis plants (e.g. within 1 week, alternatively 5 days, alternatively 3 days, alternatively 2 days, alternatively 1 day, alternatively the same day, the cannabis plant is harvested. The cannabis material may be a wet or dry biomass during the juicing process selected, and may be combined with one or more juicing additives such as a weak acid. Suitable weak acids include those partially dissociated into ions in an aqueous solution or water. A strong acid, in comparison, fully dissociates into its ions in water. Examples of a weak acids include acetic acid, formic acid, hydrocyanic acid, hydrofluoric acid, hydrogen sulfide and trichloracetic acid. While these other weak acids may not be suitable for use in food products, they yet demonstrate the appropriate strength of acid for the use in the juicing process. In specific embodiments, the juicing process includes the utilization of a food-grade weak acids, such as one or more of those that do not alter the flavor profile of the cannabis juice being prepared.

The cannabis material may be dried or undried, freshly cut, frozen-thawed, etc. prior to juicing. Likewise, the cannabis juice may be filtered (e.g. to remove chlorophyll, waxes, and inert components that may adversely affect a desired function or flavor of the juice.

In some embodiments, the cannabis extract comprises, alternatively consists essentially of, a cannabis juice concentrate, i.e., a concentrated form of the cannabis juice described above. In specific embodiments, the cannabis extract comprises a hemp juice concentrate. Typically, the cannabis juice is obtained from the upper aerial portions of immature cannabis plants.

The cannabis juice may be utilized in any amount in the cannabis composition. For example, in certain embodiments, the cannabis juice is utilized in an amount of from 1 to 50 wt. %, such as from 2 to 50, alternatively from 5 to 50, alternatively from 10 to 50, alternatively from 15 to 50, alternatively from 20 to 50, alternatively from 20 to 45, alternatively from 20 to 40 wt. %, based on the total weight of the cannabis composition.

### Extract Combinations

As will be understood by those of skill in the art, the cannabis composition may comprise but one cannabis extract, or may instead be formed as a combination of separate cannabis extracts. For example, in certain embodiments, the cannabis composition is a combination of multiple, separate extracts prepared from a single Cannabis plant variety. In some such embodiments, each of the separate extracts may have different cannabinoid content, and thus may be mixed or blended together (e.g. prior to formulation) to produce the cannabis composition therefrom. In other embodiments, the cannabis composition is a combination of multiple, separate extracts prepared from different Cannabis plant varieties.

In certain embodiments, the cannabis composition comprises a combination of cannabis powder, cannabis juice or juice concentrate, and an aqueous/alcohol-based cannabis extract composition. In specific embodiments, the cannabis composition comprises a combination of hemp powder, hemp juice or juice concentrate, and an aqueous/alcohol-based hemp extract composition. In these embodiments, the hemp powder may act as a drying agent or carrier. In these or other embodiments, the combination of cannabis extracts may comprise a separate carrier. Examples of such carriers are described further below, and are exemplified by microcrystalline cellulose, dicalcium phosphate, maltodextrin, croscarmellose sodium, silicon di-oxide, acacia, corn starch, hydroxypropyl methylcellulose, modified food starch, magnesium stearate, sugarcane fiber, sucrose, sodium alginate, pea starch, corn starch, soy protein isolate, carnauba wax, soybean oil, etc. In some embodiments, the carrier is selected from maltodextrin, pectin, gum arabic, tara gun, starch, SiO₂, and combinations thereof. In specific embodiments, the combination of cannabis extracts further comprises an additional component. Such additives are exemplified by Echinacea purpurea/e. angustifolia, turmeric, ginger, rosemary, other cannabis isolates (e.g. CBD, CBG), other phytocannabinoids, terpenes/terpenoids, szechuan peppercorn, botanicals containing n-isobutyl alkylamides or similar, galangal (root), peppers, chrysanthemum/feverfew, grain of paradise etc. (aframomum spp.), etc., and combinations thereof.

As introduced above, the cannabis composition may comprise additional components aside from the cannabis extract(s). For example, notwithstanding the additives above, the cannabis composition may comprise any of the non-cannabis phytocompounds (e.g. non-cannabis phytonutrients) introduced above. Additionally, the cannabis composition may comprise one or more carriers (e.g. pharmaceutically and/or nutritionally acceptable carriers, diluents, solvents, excipients, etc.), such as any of those described below or elsewhere herein. In some embodiments, however, the cannabis composition consists essentially of the cannabis extract, or combination of cannabis extracts set forth above. In these or other embodiments, the cannabis composition consists of, alternatively consists essentially of, the cannabis extract, optionally the non-cannabis phytocompounds, and optionally the carrier vehicle.

In certain embodiments, the cannabis composition comprises the carrier (i.e., a carrier vehicle). Suitable carrier vehicles and components can include water (e.g. purified, deionized, etc.); organic solvents such as alcohols (particularly lower alcohols readily capable of evaporating from the skin such as ethanol), glycols (such as propylene glycol, pentylene glycol, butylene glycol, and glycerol (glycerin)), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, triglycerides, sphingolipids, and waxes; protein-based materials such as collagen and gelatin; silicone-based materials (both non-volatile and volatile) such as cyclomethicone, dimethiconol, and dimethicone copolyol; hydrocarbon-based materials such as petrolatum, hydrogenated polyisobutene, and squalane; emollient esters (such as diisobutyl adipate and caprylates), thickening agents (acrylates (carbomers), acrylamides, acryl taurates, hydroxyethylcellulose, methyl cellulose, xanthan gum, etc.), and the like, as well as derivatives, modifications, and combinations thereof. Typically, the carrier vehicle is selected to dissolve or suspend the components of the cannabis composition, and thus will be selected based on the solubility of the various phytocompounds there. However, the cannabis composition may also or instead be prepared as an emulsion, suspension, slurry, etc.

### Additional Phytocompounds: Cannabimimetics

In some embodiments, the cannabis composition comprises one or more additional phytocompounds known as cannabimimetics, i.e., a non-cannabinoid plant extract or phytochemical with activity toward a cannabinoid receptor (e.g. CB1, CB2). Those of skill in the art will appreciate that the term “phytocannabinoid” has been used in a context inclusive of cannabimimetic compounds (e.g. in previously published literature), yet this term in now widely accepted as being suitable only for structural cannabinoids (e.g. CBD, THC, etc.) found in Cannabis sativa L., such that it is exclusive of cannabimimetics. Cannabimimetics may be classified as cannabis phytocompounds or non-cannabis phytocompounds, as defined above.

For example, cannibimimetics are known to occur in several plant species besides cannabis, such as Echinacea (purpurea, angustifolia), Acmella oleracea, Helichrysum umbraculigerum, marginata, etc. Phytocannabinoids obtained from such plants include lipophilic alkamides (i.e., alkylamides) from various Echinacea species, such as the isomers (cis/trans) of dodeca-2E,4E,8Z,10E/Z-tetraenoic-acid-isobutylamide, as well as other such alkylamides possessing affinity for the CB2 receptor. Other examples include the kavalactones found in the Kava plant (e.g. yangonin), which show significant affinity to the CB1 receptor); the catechins present in tea (Camellia sinensis), which show affinity for human cannabinoid receptors CB1 and CB2; and perrottetinene, which is a moderately psychoactive cannabinoid isolated from certain Radula varieties. Cannabimimetic terpenes, such as β-Carophyllene, can also be found in cannabis and non-cannabis plant species. Additional examples of cannabimimetics suitable for use in the cannabis composition, as well as representative sources, modes of action, and potential uses thereof in the cannabis composition, are set forth in the table below.

TABLE 1
Examples of cannabimimetics- phytochemicals targeting
ECS with phytocannabinoids-like activity.

| | | | | Potential |
|---|---|---|---|---|
| | Chemical | Plant | Mechanism | Therapeutic |
| Ingredient | Classification | source(s) | of Action | actions |
| Limonene | Terpene | Citrus fruits, | Glutathione | Antioxidant, |
| | | Cannabis | upregulation | antitumor |
| | | | | activity |
| β-Carophyllene | Sesquiterpene | Cannabis, Hops, | CB2 receptor | Anxiolytic, |
| (BCP) | | Copaiba, black | agonism | antinociceptive |
| | | pepper, rosemary, | | |
| | | hemp essential oil | | |
| Echinacea | Alkylamides | Echinacea, | CB2 receptor | Anti- |
| | | Sichuan pepper | agonism | inflammatory |
| | | | | antimicrobial, |
| | | | | antioxidant |
| Turmeric | Curcuminoids | Turmeric | CB1 receptor | Anti- |
| | | | agonism | inflammatory & |
| | | | | antinociceptive |
| | | | | properties |
| Boswellic acids | Triterpenes | Frankincense | Inhibition of PGE2 | Anti- |
| | | | (prostaglandin E2) | inflammatory |
| | | | synthase | |
| Magnolia | Polyphenols | Magnolia bark | CB2 receptor | Antioxidant, |
| | | | agonism | anti- |
| | | | | inflammatory |
| Ashwagandha | Lactones and | Withania | Potential GABA | Immunomodulatory, |
| | steroidal alkaloids | Somnifera | mimetic action | stress reduction |

Such cannabimimetics may be purchased, prepared, or otherwise obtained, and may be used in pure forms (i.e., forms consisting of, alternatively consisting essentially of, the cannabimimetic) or in mixtures such as botanical extracts.

### Functional Composition

As introduced above, a functional composition is also provided. More specifically, as will be appreciated from this disclosure, the cannabis composition may be formulated (i.e., as the functional composition) to prepare various consumer compositions suitable for administration to a subject, e.g. to provide one or more components of the cannabis extract thereto. In this context, the terms “administer,” “administering,” or “administration” are used herein in their broadest sense to refer to any method of delivering the functional composition, as described herein, to the subject. For example, as described further below, the functional composition may be provided in the form of a consumable composition (e.g. such as a supplement) or a personal care product (e.g. a topical composition). Food supplements are generally administered orally to the subject, whereas topical compositions are generally administered topically. It will be appreciated that the functional composition may also be provided other forms, such as medicaments, foodstuffs, animal products, home care products, etc.

The subject is typically an animal, such as a mammal (i.e., vertebrates of the class Mammalia, such as dogs, cats, goats, sheep, pigs, cattle, horses, donkeys, camels, and the like). Additional mammals that are specifically contemplated herein include semi-domesticated mammals and mammals that are routinely bred in captivity. Of course, the term mammal also encompasses humans (which may be referred to as “people” and/or “person(s)”). When describing a human, the term “adult” is typically used herein to refer to a human that has reached sexual maturity. By contrast, the terms “child” and “juvenile” are used herein to refer to a human that has not yet reached sexual maturity. Typically, the term “child” means a human subject between the stage of birth and the age of about 10 (i.e., childhood), and the term “juvenile” means a human subject that is greater than the age of about 10 and who has not completed the stage of puberty. Of course, the terms child, juvenile, adult, and infant are all encompassed by the term human, which is itself a subcategory of mammal, which is a subcategory of animal as defined herein. The subject may be a specific subset or population of the types described above. For example, in certain embodiments, the subject may be characterized as an elderly person (i.e., at least 60, alternatively at least 65, alternatively at least 70, alternatively at least 75 years of age), an athlete, a balding-male, a new mom, a student, a person with acne-prone skin, a teenager, late-teen, or pre-teen, etc.

### Biological Activity/Type

As introduced above, the functional composition is generally adapted to provide one or more components of the cannabis composition to a subject, e.g. to achieve a specific effect. More specifically, the component(s) of the cannabis extract may be utilized to mediate a particular therapeutic and/or prophylactic effect, such that the functional composition comprising the same may be used to treat or ameliorate a condition in the subject. As such, it will be understood that, in general, the functional composition comprises an effective amount of the cannabis composition or extract thereof (i.e., is formulated to provide a subject with an effective amount, alternatively a therapeutically effective amount of, the bioactive cannabis phytocompound of the cannabis extract).

The term “therapeutically effective amount” as used herein refers to its meaning as is generally accepted in the art. Specifically, the term generally refers to the amount of the functional composition that will elicit the desired and/or requisite biological response in the subject. For example, if a given treatment is considered effective when there is at least about a 25% reduction in a measurable parameter associated with a disease or disorder, a therapeutically effective amount of a drug for the treatment of that disease or disorder is that amount necessary to effect at least about a 25% reduction in that parameter.

As used herein, the terms “treatment” or “treating” may be used interchangeably, and refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### Medical Conditions (for Support/Amelioration)

For purposes of the embodiments herein, general examples of particular conditions and/or diseases may include Acquired Hypothyroidism, Acute Gastritis, Agoraphobia, AIDS Related Illness, Alcohol Abuse, Alcoholism, Alopecia Areata, Alzheimer's Disease, Amphetamine Dependency, Amyloidosis, Amyotrophic Lateral Sclerosis (ALS), Angina Pectoris, Ankylosis, Anorexia, Anorexia Nervosa, Anxiety Disorders, any chronic medical symptom that limits major life activities, any Chronic Medical Symptom that Limits Major Life Activities, Arteriosclerotic Heart Disease, Arthritis, Arthritis (Rheumatoid), Arthropathy, gout, Asthma, Attention Deficit Hyperactivity Disorder (ADD/ADHD), Autism/Asperger's, Autoimmune Disease, Back Pain, Back Sprain, Bell's Palsy, Bipolar Disorder, Brain Tumor, Malignant, Bruxism, Bulimia, Cachexia, Cancer, Carpal Tunnel Syndrome, Cerebral Palsy, Cervical Disk Disease, Cervicobrachial Syndrome, Chemotherapy Chronic Fatigue Syndrome, Chronic Pain, Chronic renal failure, Cocaine Dependence, Colitis, Conjunctivitis, Constipation, Crohn's Disease, Cystic Fibrosis, Damage to Spinal Cord Nervous Tissue, Darier's Disease, Degenerative Arthritis, Degenerative Arthropathy, Delirium Tremens, Dermatomyositis, Diabetes, Diabetic Neuropathy, Diabetic Peripheral Vascular Disease, Diarrhea, Diverticulitis, Dysthymic Disorder, Eczema, Emphysema, Emphysema, Endometriosis, Epidermolysis Bullosa, Epididymitis, Epilepsy, Felty's Syndrome, Fibromyalgia, Friedreich's Ataxia, Gastritis, Genital Herpes, Glaucoma, Glioblastoma Multiforme, Graves' Disease, Cluster Headaches, Migraine Headaches, Tension Headaches, Hemophilia A, Henoch-Schonlein Purpura, Hepatitis C, Hereditary Spinal Ataxia, HIV/AIDS, HIV-Associated Sensory Neuropathy, Hospice Patients, Huntington's Disease, Hypertension, Hypertension, Hyperventilation, Hypoglycemia, Impotence, Inflammatory autoimmune-mediated arthritis, Inflammatory Bowel Disease (IBD), Insomnia, Intermittent Explosive Disorder (IED), Intractable Pain, Intractable Vomiting, Lipomatosis, leukemia, Lou Gehrig's Disease, Lyme Disease, Lymphoma, Major Depression, Malignant Melanoma, Mania, Melorheostosis, Meniere's Disease, Motion Sickness, Mucopolysaccharidosis (MPS), Multiple Sclerosis (MS), Muscle Spasms, Muscular Dystrophy, Myeloid Leukemia, Nail-Patella Syndrome, Nightmares, Nausea, Obesity, Obsessive Compulsive Disorder, Opiate Dependence, Osteoarthritis, Panic Disorder, Parkinson's Disease, Peripheral Neuropathy, Peritoneal Pain, Persistent Insomnia, Porphyria, Post Polio Syndrome (PPS), Post-traumatic arthritis, Post-Traumatic Stress Disorder (PTSD), Premenstrual Syndrome (PMS), Prostatitis, Pruritus, Psoriasis, Pulmonary Fibrosis, Quadriplegia, Radiation Therapy, Raynaud's Disease, Reiter's Syndrome, Restless Legs Syndrome (RLS), Rheumatoid Arthritis, Rheumatoid Arthritis, Rheumatoid Arthritis, Rosacea, Schizoaffective Disorder, Schizophrenia, Scoliosis, Sedative Dependence, Seizures, Senile Dementia, Severe Nausea, Shingles (Herpes Zoster), Sinusitis, Skeletal Muscular Spasticity, Sleep Apnea, Sleep Disorders, Spasticity, Spinal Stenosis, Sturge-Weber Syndrome (SWS), Stuttering, Tardive Dyskinesia (TD), Temporomandibular joint disorder (TMJ), Tenosynovitis, Terminal Illness, Thyroiditis, Tic Douloureux, Tietze's Syndrome, Tinnitus, Tobacco Dependence, Tourette's Syndrome, Trichotillomania, Viral Hepatitis, Vomiting, Wasting Syndrome, Whiplash, Wittmaack-Ekbom's Syndrome, Writers' Cramp, and combinations thereof. In specific embodiments, e.g. where the functional composition is tailored for the use in ameliorate specific conditions, such conditions may be specified and described in further detail. However, it will be appreciated that different conditions may be beneficially treated in similar ways, such that conditions not expressly described may be equivalents with regard to the activity of the functional composition.

### Molecular Targets

In general, the functional composition be used to ameliorate conditions, such as those introduced above, by delivering bioactive cannabis phytocompounds to the subject (i.e., to mediate, induce, or elicit a positive effect in the subject). For example, in certain embodiments, the method may be used to ameliorate a condition via actions recognized as or concerned with anti-inflammatory, antioxidant, neurologic, anti-nausea/vomiting, anti-seizure, energy improvement, focus improvement, memory restoration/improvement, anti-depression, anti-anxiety (anxiolytic), mood-stabilizing, neuroprotectant, blood glucose and/or lipid level modulating (e.g. cholesterol lowering), sedative, analgesic, appetite suppressing, vasodilating, microbiome modulating, hair growth modulating, anti-cancer (e.g. cytostatic, cytotoxic, etc.), wound healing, skin restoring and/or protecting, pain-relieving, skin healing, anti-acne, and/or anti-aging.

In view of the above, it will be appreciated that the functional composition may be used to ameliorate a condition affecting a subject's skin health (e.g. melanogenesis/pigmentation), cognition (e.g. memory), mood, hormone balance, digestive health, heart health, eye health (e.g. visual function), liver health, joint and/or bone health, nutrient metabolism, elevated cholesterol, immunity, endurance, recovery, energy, stress, etc. For example, the method may be used to ameliorate such a condition by improving visual function, reducing inflammation (e.g. via inhibiting proinflammatory enzymes such as lipoxygenase (LPO), cyclo-oxygenases such as cyclooxygenase-2 (COX-2), etc.), reducing oxidant concentration, increasing vasodilation, controlling blood glucose and/or lipid levels, inhibiting a stage of a cancer process (e.g. by increasing apoptosis and/or decreasing metastasis, signal transduction, transcription factor activity, cell adhesion, etc.), and the like, or combinations thereof.

In certain embodiments, the functional composition is formulated to achieve a specific biological effect, e.g. via the activity of the component(s) of the cannabis extract used therein. For example, as described above, CBG has been shown to relieve intraocular pressure. As such, the functional composition may be formulated to include CBG with an intended use involving ameliorating glaucoma. Similarly, various components of the cannabis extract possess activity (direct or indirect) for other biological targets as described herein.

In some embodiments, the functional composition is formulated to ameliorate a condition by inhibiting or reducing the activity of inflammation-associated transcription factors and associated enzymes (e.g. upon administration of the functional composition thereto). For example, in certain embodiments the functional composition is formulated to activate peroxisome proliferator-activated receptor gamma (PPARG) (e.g. via binding by a phytocannabinoid of the functional composition thereto). Specifically, the phytocannabinoids CBD, THCA, CBDA, CBGA, and THC have each been shown to activate PPARG in in vitro and in vivo models. As such, in these embodiments, the functional composition may be formulated with one or more of these phytocannabinoids, and thus utilized to activate PPARG to promote anti-inflammatory activity and other PPARG-mediated effects, such as increased insulin sensitivity. In these or other embodiments, the functional composition is formulated to ameliorate a condition by inhibiting or mediating nuclear factor kappa-light-chain-enhancer of activated B cells (NF-κβ) in cells of the subject. NF-κB can stimulate the production of inflammatory cytokines, and misregulated NF-κB activity has been linked to inflammatory and autoimmune diseases, improper immune development, and cancer. It is believed that certain cannabis phytocompounds that may be selected for use in the functional composition (e.g. via selection of the cannabis extract thereof) possess NF-κB-inhibitory and/or mediating activity, and thus may be utilized to ameliorate conditions associated with NF-κB.

As introduced above, and described in further detail below, the functional composition may be formulated as a topical composition, e.g. for topical administration to a subject. As such, in certain embodiments, the functional composition is formulated based on a specific biological activity that may be achieved via topical application and/or transdermal delivery methods in particular. For example, in specific embodiments, the functional composition is formulated to treat cutaneous malignancies such as melanoma and non-melanoma skin tumors. In these or other embodiments, the functional composition is formulated to treat another skin condition.

Skin is the largest organ of the human body and serves as a protective barrier against environmental insults, which can lead to the generation of reactive oxygen species (ROS). Accumulation of harmful ROS that induce cell damage via oxidative stress may lead to chronic inflammation and skin disorders, which can be countered in healthy skin by activation of certain defense mechanisms controlled by nuclear factor erythroid 2-like 2 (NRF2) and PPARG. Under regular conditions, NRF2 is maintained at low levels by the negative regulator, KEAP1, although NRF2-KEAP1 binding is impaired by cellular exposure to ROS. As such, ROS build up may lead to translocation and binding of NRF2 to antioxidant response elements (AREs) in the nucleus, resulting in activation of NRF2 target genes. The stress-induced enzyme Hemeoxygenasel (HMOX1) is amongst one of the NRF2 target genes and exhibits antioxidant and anti-inflammatory properties. HMOX1 is one of the widely used biomarkers for stress response as it is highly induced by various stressors like UV radiation, nitric oxide, chemicals, heavy metals, and oxidative stress. The expression of HMOX1 is positively regulated by NRF2 and is negatively regulated by BACH1 gene (BTB and CNC Homology 1).

It is believed that CBD may upregulate HMOX1 and induce the expression of other NRF2-dependent genes in skin cells. Moreover, CBD has been shown to inhibit BACH1 and weakly activate NFR2. As such, with HMOX1 and PPAR-γ playing strong cytoprotective roles involving anti-inflammatory, antioxidant, and anti-apoptotic properties, it is believed that CBD, as well as other components of the functional composition, may be utilized to ameliorate conditions relating to inflammation and/or oxidation by mediating the NRF2-involved enzyme pathways above. As such, in some embodiments, the functional composition is formulated with one or more cannabis phytocompounds with activity for activating or upregulating NRF2 and/or HMOX1, inhibiting BACH1 and/or KEAP1, or combinations thereof.

In certain embodiments the functional composition is formulated to ameliorate a chronic pain condition via peripheral inflammatory action, upon topical administration to a subject.

Tissue damage typically triggers an inflammatory response in the body that can result in irritation, ulcers, sensitization of peripheral tissues, neuropathies, and chronic wounds. If left unresolved, a chronic inflammatory state in the body leads to increased tissue damage and pain. Current therapies for chronic pain management (e.g. antidepressants, NSAIDs, anticonvulsants) target the peripheral and central nervous system (CNS) and often produce undesirable side effects. As such, in some embodiments, the functional composition comprises one or more topically-bioavailable cannabis phytocompounds with targeted activity against peripheral inflammation to reducing pain for specific conditions while circumventing the CNS. It is believed that the functional composition, so configured, will exhibit fewer side effects i.e., respiratory depression, sedation, and tolerance, as compared to conventional therapies.

In certain embodiments the functional composition is formulated to ameliorate a skin condition. In some such embodiments, the skin condition is eczema. In other such embodiments, the skin condition is atopic dermatitis, pruritis (itch), acne, or psoriatic plaque.

Atopic dermatitis is a chronic inflammatory skin disorder associated with multifactorial causes like environmental triggers, damaged skin barrier function, microbiome imbalance, genetic predisposition and an altered immune response. Certain cannabis phytocompounds have been shown to modulate inflammatory responses by regulating more than one underlying mechanism. For example, adelmidrol, a PEA derivative, has been shown to be effective in treating mild atopic dermatitis in a pediatric population. As such, it is believed that CBD, in isolation and/or in combination with one or more other cannabis phytocompounds, may exhibits anti-inflammatory properties, such as those associated with allergic contact dermatitis. Accordingly, in certain embodiments the functional composition is formulated to ameliorate allergic contact dermatitis upon topical administration to a subject.

Specifically, the influence of microbiome imbalance, especially due to colonization and biofilm formation of Staphylococcus aureus, has emerged as an influencing factor which can contribute towards the severity of dermatitis. The microorganisms in biofilms live in a self-produced matrix. In most biofilms, the live microorganisms account for less than 10% of the dry mass, whereas the matrix can account for over 90%. The matrix is the hydrated extracellular polymeric substances (EPS) in which the biofilm cells are embedded. EPS mainly contain polysaccharides, proteins, nucleic acids and lipids to provide the mechanical stability of biofilms and mediate their adhesion to the surfaces. EPS forms a cohesive, three-dimensional polymer network that interconnects and immobilizes biofilm cells.

Staphylococcus aureus is a Gram-positive bacterium that colonizes naturally or transiently on human skin and is a causal organism of many skin disorders (e.g., dermatitis, rosacea, psoriasis). It is also associated with several nosocomial infections such as prostatitis, endocarditis, and osteomyelitis. Persistence and resistance of S. aureus is due to its capability to develop a biofilm that promotes antibiotic tolerance and protection from the host immune system, thus making it difficult to eradicate.

It is believed that the functional composition may exhibit antimicrobial and antibiofilm activity when formulated with an essential oil (EO; steam distillate) fraction of hemp. Such a fraction may be comprised mainly of terpenoids, such as myrcene, α-pinene, β-caryophyllene and other terpenes as described above, with or without significant levels of CBD. Reported data suggests that effective Minimum Inhibitory Concentration (MIC), Minimum Bactericidal Concentration (MBC) and Minimum Biofilm Eradication Concentration (MBEC) of hemp EO values against all S. aureus strain types have been reported as 8, 16 and 24 mg/mL, which indicated that hemp EO can be utilized to eradicate a mature biofilm of S. aureus. Thus, based on the antimicrobial and antibiofilm activities of hemp EO against S. aureus, it is believed that the functional composition provides therapeutic potential to prevent skin disorders like atopic dermatitis.

Itch (pruritis) is an unpleasant sensation of skin with a desire to scratch. When it becomes chronic, it can severely affect one's quality of life. The pathogenesis of pruritis is well researched. Though most of the ECS research indicates that the itch response is primarily modulated through CB1 receptors in the central (brain and spinal) system, recent reports suggest the involvement of peripheral CB1 receptors could be also be a potent contributor to itch. It has also been shown that all ionotropic cannabinoid responsive receptors (e.g., TRPV1-4, TRPA1 and TRPM8) play a vital role in the complex cutaneous communication between keratinocytes, immune (Mast) cells and the sensory nerves which leads to an itch sensation. Thus, inhibiting the activity of such ionotropic channels may be helpful in alleviating pruritis.

For example, FAAH and MAGL inhibitors, which can increase the levels of endocannabinoids and modulate cannabinoid and non-cannabinoid receptor responses, were found to demonstrate anti-pruritic effects on murine models when administered via intra peritoneal and intrathecal routes. Though cannabinoids like THC and PEA have been shown to reduce itching in murine models, the human clinical data for testing the antipruritic potential of PEA has resulted in conflicting results. Moreover, some peripheral endocannabinoids have opposite effects on itching behavior in spinally versus trigeminally innervated skin of mice, and therapeutic treatment of itch might be more relevant for treating the lower body than itch arising from trigeminal innervated skin of face or scalp. Since CBD is a FAAH inhibitor, a CB2 inverse agonist (antagonist of CB2 agonists) and TRPV1 agonist, it is believed that the functional composition may be utilized to modulate itch response though this mechanism.

The major factors involved in acne onset are sebum overproduction, unwanted sebocyte proliferation, and inflammation. It is known that the ECS plays a key role in homeostasis of the skin, and specifically in lipogenesis. For example, the endocannabinoid, anandamide (AEA), has been shown to stimulate lipid production in human sebocytes at low concentrations, but induces apoptosis at higher levels. It is believed that CBD could be a novel therapeutic in the management of acne by acting on pathways relating to sebum production, sebocyte proliferation, and inflammation.

Additionally, imbalance in the skin microbiome may also contribute to the pathogenesis of acne. Specifically, Cutibacterium acnes (C. acnes) has been linked to the establishment of acne for over 100 years. Therefore, the functional composition, which may exhibit anti-microbial effects of when formulated with CBD and/or other antimicrobial cannabis phytocompounds, may also prove effective in acne treatment. In specific embodiments, hemp seed extract or hemp EO may be used for treating acne vulgaris due to its anti-lipogenic, anti-proliferative, anti-inflammatory, and anti-microbial, properties, which may target similar or independent mechanisms than that of CBD.

Psoriatic plaques are characterized by keratinocyte hyperproliferation and chronic inflammation. NF-kB plays a significant role in skin inflammatory conditions like psoriasis, and its expression is strongly induced by TNF-α. In addition to the TNF-α induced NFkB transcription inhibitory properties of CBD described above, CBD has been reported to have the ability to impair the NF-kB pathway both in-vitro and in-vivo. As such, it is believed that the functional composition, may be used in the treatment for psoriatic lesions.

In certain embodiments the functional composition is formulated to facilitate wound healing. Wound healing is an intricate process which includes three overlapping phases—inflammation, proliferation, and maturation/tissue remodeling. Upon skin injury, the wound is infiltrated by various immune cells resulting in release of various growth factors and cytokines which promote the inflammatory process. During the proliferation phase, fibroblasts are involved in synthesizing extracellular matrix (ECM) whereas keratinocytes proliferate and migrate close to edges of the wound. ECM remodeling happens in the final stages to restore the barrier integrity of the tissue. Though both fibroblasts and keratinocytes are involved in inflammation and wound healing response, fibroblasts are primarily involved in ECM remodeling and keratinocytes are mainly involved in the inflammatory process.

It is believed that the complex process of wound healing is influenced by ECS signaling, as it modulates epidermal proliferation and differentiation, fibroblast functions, and cutaneous inflammation. CB1 and CB2 receptor involvement during the wound healing process in various immune and fibroblast cells are based on murine models. Various cannabinoid analogs are known to generate a wound healing response in relevant models, possibly due to activation of CB1 and/or CB2 receptors, upregulation of anti-inflammatory factors, indirect activation of TRPV1 and epidermal growth factor receptors, and inhibition of the FAAH enzyme. For example, reported effects of CBD and a standardized 5% CBD extract on human keratinocytes (HaCaT cells) and human dermal fibroblast (HDF) showed that TNF-α treatment resulted in upregulated expression of 26 genes involved in inflammatory pathways and included chemokines like CXCL8 and CXCL10, interleukins like IL-17C and IL-1B, and VEGF-A. Treatment with the extract downregulated all 26 inflammatory related genes, and CBD alone downregulated 15 genes. In HDF cells, TNF-α treatment upregulated 16 genes involved in the process of wound healing. While CSE was again able to downregulate all genes, CBD only downregulated 11 genes and did not exhibit any inhibitory effects on genes playing a role in inflammation and matrix remodeling, including IL-6 and MMP-9. As such, it is believed that the components of the functional composition provided by the cannabis extract, e.g. the cannabinoids, flavonoids, and terpenes, etc. may exert a synergistic anti-inflammatory effect greater than that of CBD alone, and may thus provide an improved composition for use in wound healing.

In certain embodiments the functional composition is formulated to modulate human hair growth. Specifically, as the hair follicle contains ECS and cannabinoid-responsive receptors, and cannabinoid deposition within the fiber has been demonstrated following cannabis consumption and topical application, it is believed that the functional composition may be used to treat certain hair disorders, such as alopecia or hirsutism.

The human hair follicle is an immune-privileged miniaturized organ consisting of epithelial and mesenchymal tissue. As part of the pilosebaceous complex, the hair follicle is extensively regulated, the extent of which is still not completely understood. Human scalp hair growth is a complex and dynamic process including a period of keratinocyte proliferation and hair fiber growth (anagen), followed by a stage of apoptotic follicle regression (catagen) and a semi-quiescent stage (telogen). A complete cycle has a duration of two-six years with a majority of hair follicles in the anagen phase for a large part of the cycle.

The command center of the hair follicle, the dermal papilla (DP), in conjunction with the bulge, controls the activation of new growth cycles through dynamic processes. Stem cell populations from both regions, through DP control, work to regenerate hair follicle structure and initiate keratinocyte proliferation in a new anagen cycle. Hair growth abnormalities include lack of hair growth (alopecia), and excessive hair growth (hirsutism and hypertrichosis). Alopecia causation can be due to alterations in hair follicle cycling (extended telogen), hormonal (androgenic alopecia), autoimmune disorders (alopecia areata), infection (tinea capitis), or prolonged inflammation. Hirsutism is a female condition causing excessive hair growth in androgen-dependent tissues, while hypertrichosis can affect males and females of varying age and ethnicity being widespread or localized on the body.

Immunohistochemical analysis of human skin revealed differential expression of CB1 and CB2 receptors within the hair follicle. CB1 was detected in portions of the infundibulum and the inner root sheath, but absent from the outer root sheath, the bugle, hair bulb, and arrector pili muscle. CB2 was present in the outer root sheath and hair bulb, but absent in the inner root sheath, bulge, and arrector pili muscle. Surgically isolated facial hair follicle cultures showed endocannabinoid (anandamide and 2-arachidonoylglycerol) production Intriguingly, anandamide, and (9)-tetrahydrocannabinol suppressed hair follicle growth and induced the catagen cycle. The effects of these endo-exo cannabinoids were ameliorated by the addition of a CB1 antagonist. 2-arachidonoylglycerol treatment, however, resulted in comparable follicle growth to control-treated follicles.

The expression of TRPV1 in human hair follicles and outer root sheath keratinocytes. Activation of TRPV1 in follicle organ cultures has been demonstrated to lead to inhibition of cell proliferation, while inducing apoptosis and catagen entry. Hair growth activators, HGF, IGF1, and SCF, were also suppressed with TRPV1 stimulation. TRPV3 and TRPV4 were also detected in human hair follicles and outer root sheath keratinocytes, and receptor activation results in suppression of hair follicle elongation. A metabolite of the endocannabinoid anandamide, bimatoprost, is recognized as a topical prostamide treatment for eyebrow hypotrichosis. Khidhir et. al. also showed human scalp hair follicles possess select prostamide receptors within the dermal papilla. Working with human scalp organ-cultured hair follicles bimatoprost treatment resulted in follicle growth and it stimulated hair regrowth when applied to mouse skin. Recent work has also shown that bimatoprost application accelerates hair regrowth in alopecia areata patients to a greater extent than a topical steroid treatment, and studies using ex vivo human hair follicles and primary outer root sheath keratinocytes have found systemic-like application of CBD had dose-dependent opposing effects on hair growth dynamics. As such, it is believed that the components of the functional composition provided by the cannabis extract, may favorably affect hair growth pathways.

In certain embodiments the functional composition is formulated to modulate human skin and/or hair pigmentation. Specifically, the pigmentation of human skin is the manifestation of synthesis of dark pigment, melanin, which is regulated by a melanogenesis process in melanocytes. Melanogenesis is a complex process regulated by more than 250 genes. Microphthalmia Transcription Factor (MITF) acts as a master regulator of melanogenesis, directly controlling the transcription of key genes involved in pigmentation such as TYR, TYR-1, and TYR-2.150 Regulation of gene expression of MITF takes place via various signaling pathways such as a-MSH/MCIR, Wnt/β-catenin, and SCF/c-Kit (ERK signaling pathway). Melanocortin-1 receptor (MCIR) is the well-known G protein coupled receptor which regulates t melanin synthesis via its agonist, a-MSH and antagonist ASP. The Wnt signaling pathway is initiated by the binding of Wnt molecule to a Frizzled receptor while the activation of extracellular signal-regulated kinase (ERK) results in the ubiquitin-mediated proteosomal degradation of MITF.

It has been shown that a fully functional ECS is present in normal human primary epidermal melanocytes. Moreover, lower concentrations of AEA, as well as other endocannabinoids like ACEA (Arachidonoyl-2′-chloroethylamide), and 2-AG, demonstrate induction of melanogenesis in dose-dependent manner via the CB1 receptor. However, CB1 agonism may inhibit melanogenesis, or having no influence. For example, OEA acts as an inhibitor of melanin synthesis and MITF production in a-MSH stimulated B16 cells via activation of ERK, Akt, and p38 pathways and inhibition of the CREB pathway. Additionally, CB1 agonism, under UVB exposure in a co-culture model using HaCat and SK-mel-1 cells, has been shown to inhibit basal melanogenesis. As such, potential melanogenic effects of cannabidiol occurs primarily through MITF upregulation, which is mediated by the activation of p42/44 MAPK and p38 MAPK signaling. Accordingly, it is believed that the functional composition may be utilized to mediate healthy and diseased skin, such as for the treatment of a pigmentation disorder.

General Components (of the Functional Composition)

In general, the functional composition is not limited in terms of formulation, peripheral ingredients, form, number of functions, etc., aside from comprising the cannabis composition, or cannabis extract, described herein. Rather, the functional composition may be varied, and may be formulated in any fashion consistent with this disclosure.

As introduced above, the functional composition is adapted for ameliorating a condition of, and/or to confer a health benefit to, a subject. As such, the functional composition comprises an active agent (i.e., a compound or composition capable of eliciting a particular biological effect in a subject). Typically, the active agent comprises, optionally is, the cannabis extract or one or more components (i.e., cannabis phytocompounds) thereof. However, in various embodiments, other active agents (e.g. probiotics, prebiotics, parabiotics, pharmaceuticals, nutraceuticals, anesthetics, counterirritants, chondroprotective agents, etc.) may be utilized in the functional composition in addition to the cannabis extract. For example, in certain embodiments, the functional composition comprises a pharmaceutically acceptable additive, which may comprise or be one or more of the various components described below. In other embodiments, however, the functional composition is substantially free from, alternatively free from other active agents (i.e., other than the cannabis extract and cannabis phytocompounds thereof).

Co-Actives

Examples of other active agents suitable for use in the functional composition are described below. One or more of these active agents can be used, either alone or in combination with the actives and/or additives described herein.

The functional composition may include an antiparasite agent. Examples of antiparasite agents include hexachlorobenzene, carbamate, naturally occurring pyrethroids, permethrin, allethrin, malathion, piperonyl butoxide, and combinations thereof.

The functional composition may include an antimicrobial agent, also referred to as germicidal agents. Examples of antimicrobial agents include phenols, including cresols and resorcinols. Such compositions may be used to treat infections of the skin. An example of a very common skin infection is acne, which involve infestation of the sebaceous gland with p. acnes, as well as Staphylococus aurus or Pseudomonas. Examples of useful antiacne actives include the keratolytics such as salicylic acid (o-hydroxybenzoic acid), derivatives of salicylic acid such as 5-octanoyl salicylic acid, and resorcinol; retinoids such as retinoic acid and its derivatives (e.g. cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids; and bile salts such as scymnol sulfate and its derivatives, deoxycholate and cholate; parachlorometaxylenol; and combinations thereof.

Phenols, in concentrations of 0.2, 1.0, and 1.3, % by weight, are generally bacteriostatic, bactericidal, and fungicidal, respectively. Several phenol derivatives are more potent than phenol itself, and the most important among these are the halogenated phenols and bis-phenols, the alkyl-substituted phenols and the resorcinols. Hydrophobic antibacterials include triclosan, triclocarbon, eucalyptol, menthol, methylsalicylate, thymol, and combinations thereof.

The functional composition may include an antifungal agent. Examples of antifungal agents include azoles, diazoles, triazoles, miconazole, fluconazole, ketoconazole, clotrimazole, itraconazole griseofulvin, ciclopirox, amorolfine, terbinafine, Amphotericin B, potassium iodide, flucytosine (5FC) and combinations thereof. U.S. Pat. No. 4,352,808 discloses 3-aralkyloxy-2,3-dihydro-2-(1H-imidazolylmethyl)benzo[b]thiophene compounds having antifungal and antibacterial activity, which are incorporated herein by reference.

The functional composition may include a steroidal anti-inflammatory agent. Examples of steroidal anti-inflammatory agents include corticosteroids such as hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, and combinations thereof.

Topical antihistaminic preparations currently available include 1 percent and 2 percent diphenhydramine (Benadryl® and Caladryl®), 5 percent doxepin (Zonalon®) cream, phrilamine maleate, chlorpheniramine and tripelennamine, phenothiazines, promethazine hydrochloride (Phenergan®) and dimethindene maleate. These drugs, as well as additional antihistamines can also be included in the composition. Additionally, “natural” anti-inflammatory agents may be useful. For example, candelilla wax, alpha bisabolol, aloe vera, Manjistha (extracted from plants in the genus Rubia, particularly Rubia cordifolia), and Guggal (extracted from plants in the genus Commiphora, particularly Commiphora mukul, may be used as an active in the functional composition.

The functional composition may include a non-steroidal anti-inflammatory drug (NSAID). Examples of NSAIDs include the following NSAID categories: propionic to acid derivatives; acetic acid derivatives; fenamic acid derivatives; biphenylcarboxylic acid derivatives; and oxicams. Such NSAIDs are described in the U.S. Pat. No. 4,985,459, which is incorporated herein by reference. Further examples include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, mniroprofen, tioxaprofen, suprofen, alminoprofen, tiaprofenic acid, fluprofen, bucloxic acid, and combinations thereof.

The functional composition may include an antioxidant/radical scavenger. Examples of antioxidants include ascorbic acid (vitamin C) and its salts, tocopherol (vitamin E), and its derivatives such as tocopherol sorbate, other esters of tocopherol, butylated hydroxy benzoic acids and their salts, 6-hydroxy-2,5,7,8-tetramethylchroman-2-carboxylic acid (commercially available under the trade name Trolox®), gallic acid and its alkyl esters, especially propyl gallate, uric acid and its salts and alkyl esters, sorbic acid and its salts, the ascorbyl esters of fatty acids, amines (e.g. N,N-diethylhydroxylamine, amino-guanidine), sulfhydryl compounds (e.g. glutathione), and dihydroxy fumaric acid and its salts may be used, as well as EDTA, BHT and the like, and combinations thereof.

The functional composition may include an antibiotic. Examples of antibiotics include chloramphenicol, tetracyclines, synthetic and semi-synthesic penicillins, beta-lactames, quinolones, fluoroquinolnes, macrolide antibiotics, peptide antibiotics, cyclosporines, erythromycin, clindamycin, and combinations thereof.

The functional composition may include a topical anesthetic. Examples of topical anesthetics include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, pharmaceutically acceptable salts thereof, and combinations thereof.

The functional composition may include an anti-viral agent. Examples of anti-viral agents include certain proteins, polypeptides, peptides, fusion protein antibodies, nucleic acid molecules, organic molecules, inorganic molecules, and small molecules that inhibit or reduce the attachment of a virus to its receptor, the internalization of a virus into a cell, the replication of a virus, or release of virus from a cell. In particular, anti-viral agents include nucleoside analogs (e.g. zidovudine, acyclovir, acyclovir prodrugs, famciclovir, gangcyclovir, vidarabine, idoxuridine, trifluridine, and ribavirin), n-docosanoll foscarnet, amantadine, rimantadine, saquinavir, indinavir, ritonavir, idoxuridine alpha-interferons and other interferons, AZT, and combinations thereof.

Additional examples of actives include analgesic agents and antihypertensive agents. Analgesic agents are known in the art and are colloquially referred to as painkillers. The analgesic agent may be selected from any known analgesic agents, and specific examples thereof include paracetamol (acetaminophen), morphine, codeine, heroine, methadone, thebaine, orpiarine, buprenorphine, morphinans, benzomorphans, acetaminophen, butorphanol, diflunisal, fenoprofen, fentanyl, fentanyl citrate, hydrocodone, aspirin, sodium salicylate, ibuprofen, oxymorphone, pentaxicine, naproxen, nalbuphine, mefenamic acid, meperidine and dihydroergotamine, non-steroidal anti-inflammatory agents, such as salicylates, and opioid agents, such as morphine and oxycodone. Antihypertensive agents are known in the art for treating or reducing hypertension, i.e., high blood pressure. The antihypertensive agent may be selected from any known antihypertensive agents, and specific examples thereof include diuretics, adrenergic receptor antagonists (e.g. beta blockers), benzodiazepines, calcium channel blockers, renin inhibitors, etc.

A typical narcotic antagonist is haloxone. Exemplary antitussive agents include diphenhydramine, guaifenesin, hydromorphone, ephedrine, phenylpropanolamine, theophylline, codeine, noscapine, levopropoxyphene, carbetapentane, chlorpehndianol and benzonatate.

Exemplary sedatives include chloral hydrate, butabarbital, alprazolam, amobarbital, chlordiazepoxide, diazepam, mephobarbital, secobarbital, diphenhydramine, ethinamate, flurazepam, halazepam, haloperidol, prochlorperazine, oxazepam, and talbutal.

Examples of cardiac drugs include quinidine, propranolol, nifedipine, procaine, dobutamine, digitoxin, phenyloin, sodium nitroprusside, nitroglycerin, verapamil HCl, digoxin, nicardipine HCl, and isosorbide dinitrate.

Antiemetics are illustrated thiethylperazine, metoclopramide, cyclizine, meclizine, prochlorperazine, doxylamine succinate, promethazine, triflupromazine, and hydroxyzine.

A typical dopamine receptor agonist is bromocriptine mesylate. Exemplary amino acid, peptide and protein hormones include thyroxine, growth hormone (GH), interstitial cell stimulating hormone (ICSH), follicle-stimulating hormone (FSH), thyrotropic hormone (TSH), adrenocorticotropic hormone (ACTH), gonadotropin releasing hormone (GnRH) such as leuprolide acetate, vasopressin and their active degradation products Some products may have sufficiently high molecular weights that absorption through the stratum corneum or mucous membranes may be difficult. Therefore, in some embodiments, the only hormones having molecular weights and stereo configurations allowing passage through the skin are utilized. Example of hormones include estradiol, diethylstilbestrol, conjugated estrogens, estrone, norethindrone, medroxyprogesterone, progesterone, norgestrel, testosterone, methyltestosterone, and fluoxymesterone.

In particular embodiments, the functional composition comprises a combination of the cannabis extract and the additional active agent. In such embodiments, the functional composition may be homogeneous or mixed as a unitary composition or, alternatively, may be adapted as a kit including a first component comprising the cannabis extract and a second component comprising the additional active agent. In some such embodiments, the additional active agent is a pharmaceutical agent (e.g. an angiotensin converting enzyme (ACE) inhibitor, an anti-inflammatory agent, a vasodilator, an immune modulating agent (i.e., an antibody therapeutic agent), an analgesic, an antibiotic, etc.). The components of the kit may be administered together or separately (e.g. sequentially, in any order).

### Additive Component

In addition to the components described above (i.e., the cannabis composition/extract, the pharmaceutically acceptable additive, etc.), the functional composition may comprise any number of additional ingredients/components. For example, in some embodiments, the functional composition comprises an additive component, which may comprise one or more additives.

Examples of suitable additives for use in the additive component include amino acids, peptides, proteins, lipids, vitamins, carbohydrates, nucleic acids, minerals, nutrients, antioxidants, probiotic bacterial strains, lipotropic agents, extracts, concentrates, oils, gums, and combinations thereof. In certain embodiments, the additive component comprises a flavoring agent, a dye, a flow modifier, a preservative, a filler, a binder, a dispersing agent, a solubilizer, a supplemental nutrient, an excipient, a buffer, a lubricant, a sweetener, a wetting agent, or any combination thereof. Particular examples of suitable additives include vitamin A, vitamin D, calcium, methyl cellulose, lecithin, lysolecithin, and long-chain fatty alcohols. In particular embodiments, the additive component comprises a carrier, such as a consumable, nutritional, and/or pharmaceutical carrier, or a combination thereof.

Additional examples of additives suitable for use in the additive component of the functional composition include pea protein isolate, isomalto-oligosaccharide, rice protein concentrate, 2′-fucosyllactose powder, flaxseed, organic cane sugar, natural and artificial flavors, high oleic sunflower oil, L-lysine HCl, medium chain triglycerides, L-leucine, silica, L-valine, L-alanyl-L-glutamine, L-isoleucine, xanthan gum, vitamins, minerals, zinc gluconate, ascorbic acid, manganese gluconate, alpha tocopheryl acetate, copper gluconate, D-biotin, retinyl palmitate, niacinamide, cholecalciferol, calcium pantothenate, chromium picolinate, pyridoxine HCl, riboflavin, potassium iodide, thiamin HCl, calcium L-5-methyltetrahydrofolate, selenomethionine, and methylcobalamin, Luo Han Guo fruit (monk fruit) extract, vanilla, rosemary extract, cocoa powder, vitamin E, thiamin, riboflavin, niacin, vitamin B6, folate, vitamin B12, biotin, pantothenic acid, phosphorus, iodine, magnesium, zinc, selenium, copper, manganese, and the like, and combinations thereof. Of course, components aside from those listed above concerning the additive component may also be utilized in the functional composition.

In certain embodiments, the additive component of the functional composition comprises one or more of the following: excipients, such as diluents and binders; granulating agents; glidants (or flow aids); fillers; lubricants; stabilizers; bulking agents; anti-caking agents; coatings; disintegrants; fragrances; natural or artificial sweeteners; flavorings; and pigments; alcohols, such as ethanol, propyl alcohol and benzyl alcohol; glycerin; glyceryl triacetate; mineral oils; water; silicones, such as silicone oils; silicon dioxide; waxes, such as carnauba wax and beeswax; fatty esters and fatty alcohols; carob; corn syrups, such as hydrolyzed corn syrup solids; cellulose, such as methyl cellulose, hydroxypropyl methyl cellulose, carboxy methyl cellulose, microcrystalline cellulose, and powdered cellulose; fructose; maltodextrin and maltol, such as natural maltol; sorbitol; preservatives, such as p-hydrobenzoic acid esters; potassium sorbate; sodium benzoate; flow agents; stearates, such as calcium stearate, magnesium stearate, and sodium magnesium stearate; dicalcium phosphate; vegetable oils, such as hydrogenated vegetable oils; antioxidants, such as ascorbic acid or tocopherol; starches, such as corn starch and potato starch; glycols and polyglycols; moisturizers; emollients; emulsifiers; surfactants; oils; extracts; skin protectants; disinfectants; antiseptics; drugs and drug substances; analgesic compounds; anti-neuralgic compound; anti-oxidants; blood circulation promoters; antidepressant compounds; anti-anxiety compounds; anti-stress compounds; colorants; fillers; solvents; vehicles; carriers; other types of additives known to those of skill in the art (e.g. nutraceutical and/or nutritional (e.g. food) additives); and combinations thereof.

Of course, components aside from the additive component may also be utilized in the functional composition. For example, the functional composition may comprise a fat component, a lipid component, a protein component, a fiber component, a carbohydrate component, and the like, or combinations thereof, which may be independently selected, e.g. based on the desired formulation, form, and/or end use of the functional composition, as will be understood by those of skill in the art in view of the description herein. For example, as described further below, the functional composition may be formulated for oral administration to a subject. In such embodiments, the functional composition may comprise an effective amount of one or more sweeteners, including carbohydrate sweeteners and natural and/or artificial no/low calorie sweeteners, selected based on the type and/or intensity of sweetness intensity desired.

### Plant Extracts

In some embodiments, the functional composition comprises one or more plant or vegetable extracts. Examples of these components are as follows: Ashitaba extract, avocado extract, hydrangea extract, Althea extract, Arnica extract, aloe extract, apricot extract, apricot kernel extract, Ginkgo Biloba extract, fennel extract, turmeric[Curcuma] extract, oolong tea extract, rose fruit extract, Echinacea extract, Scutellaria root extract, Phellodendro bark extract, Japanese Coptis extract, Barley extract, Hyperium extract, White Nettle extract, Watercress extract, Orange extract, Dehydrated saltwater, seaweed extract, hydrolyzed elastin, hydrolyzed wheat powder, hydrolyzed silk, Chamomile extract, Carrot extract, Artemisia extract, Glycyrrhiza extract, hibiscustea extract, Pyracantha Fortuneana Fruit extract, Kiwi extract, Cinchona extract, cucumber extract, guanocine, Gardenia extract, Sasa Albo-marginata extract, Sophora root extract, Walnut extract, Grapefruit extract, Clematis extract, Chlorella extract, mulberry extract, Gentiana extract, black tea extract, yeast extract, burdock extract, rice bran ferment extract, rice germ oil, comfrey extract, collagen, cowberry extract, Gardenia extract, Asiasarum Root extract, Family of Bupleurum extract, Salvia extract, Saponaria extract, Bamboo extract, Crataegus fruit extract, Zanthoxylum fruit extract, shiitake extract, Rehmannia root extract, gromwell extract, Perilla extract, linden extract, Filipendula extract, peony extract, Calamus Root extract, white birch extract, Horsetail extract, Hedera Helix(Ivy) extract, hawthorn extract, Sambucus nigra extract, Achillea millefolium extract, Mentha piperita extract, sage extract, mallow extract, Cnidium off icinale Root extract, Japanese green gentian extract, soybean extract, jujube extract, thyme extract, tea extract, clove extract, Gramineae imperata cyrillo extract, Citrus unshiu peel extract Japanese Angellica Root extract, Calendula extract, Peach Kernel extract, Bitter orange peel extract, Houttuyna cordata extract, tomato extract, natto extract, Ginseng extract, Green tea extract (camelliea sinesis), garlic extract, wild rose extract, hibiscus extract, Ophiopogon tuber extract, Nelumbo nucifera extract, parsley extract, honey, hamamelis extract, Parietaria extract, Isodonis herba extract, bisabolol extract, Loquat extract, coltsfoot extract, butterbur extract, Porid cocos wolf extract, extract of butcher's broom, grape extract, propolis extract, luffa extract, safflower extract, peppermint extract, linden tree extract, Paeonia extract, hop extract, pine tree extract, horse chestnut extract, Mizu-bashou [Lysichiton camtschatcese] extract, Mukurossi peel extract, Melissa extract, peach extract, cornflower extract, eucalyptus extract, saxifrage extract, citron extract, coix extract, mugwort extract, lavender extract, apple extract, lettuce extract, lemon extract, Chinese milk vetch extract, rose extract, rosemary extract, Roman Chamomile extract, royal jelly extract, and combinations thereof.

### Pre/Probiotics

In certain embodiments, the functional composition comprises a probiotic, a prebiotic, or both. The term “probiotic” as used herein can mean one or more microorganisms which, when administered appropriately, can confer a health benefit on the host or subject. Examples of suitable probiotics typically include members of the Coriobacteriaceae family and/or the Clostridium coccoides—Eubacterium rectale cluster, as well as various Lactobacillus sp. and Bifodobacterium sp. Some examples of suitable probiotics include those of family Bacteroidaceae, Clostridiaceae, Prevotellaceae, Eubacteriaceae, Ruminococcaceae, Bifidobacteriaceae, Lactobacillaceae, Enterobacteriaceae, Saccharomycetaceae, Methanobacteriaceae, and the like, or combinations thereof. Some particular examples include: C. orbiscidens; Eubacterium; oxidoreducens; B. subtilus; Bacteroides distasonis; Bacteroides uniformis; Bacteroides ovatus; Enterococcus casseliflavus; Eubacterium ramulus; Lactobacillus—Enterococcus; Lachnospiraceae; L. johnsonii; Bifidobacterium catenulatum; Bifidobacterium pseudocatenultum; Gordonibacter urolithinfaciens; Gordonibacter pamelaeae; Clostridium coccoides; Clostridium leptum; Streptococcus intermedius; Ruminococcus productus; Eggerthella sp. Julong 732; Enterococcus faecium EP11; Lactobacillus mucosae EPI2; Finegoldia magna EPI3; Feacalibacterium; Slackia isoflavoniconverten; and Eggerthella sp.; as well as various derivatives and/or combinations thereof.

In certain embodiments, the functional composition comprises the prebiotic. The term “prebiotic” as used herein refers to a compound, or a combination of compounds, that is typically not digestible by the subject (e.g. an animal), but which may selectively stimulate the growth and/or activity of one or a limited number of beneficial bacteria in the microbiome of the subject. Additionally, the term “prebiotic effect” refers to a selective, prebiotic-induced stimulation of growth and/or activity of one or a limited number of bacteria (e.g. bifidobacteria, lactobacilli, etc.) in the microbiome of the host. In general, the prebiotic is not limited, and may be any compound or combination of compounds that stimulate the growth of one or more microbes in the microbiome of the host, including those exemplified herein with respect to the probiotic. The prebiotic may stimulate such growth directly (e.g. by providing nutrients to the microbes) and/or indirectly (e.g. by preventing growth of competing microbes). Examples of suitable prebiotics typically include fibers, such as soluble fibers (i.e., those which dissolve in water) and insoluble fibers (i.e., those which do not dissolve in water). Some examples of fibers include starch, non-starch polysaccharides and oligosaccharides, carbohydrate fibers, lignans, and the like, as well as combinations thereof. Specific examples of suitable fibers for use in or as the prebiotic include cellulose, hemicellulose, arabinoxylans, polyfructose, inulin, oligofructans, galacto-oligosaccharides, gums, mucilages, pectins, dextrins, malodextrins, synthetic carbohydrates, polydextrose, methyl cellulose, hydroxypropylmethyl cellulose, waxes, phytate, cutin, saponins, suberin, tannins, chitosans, alginates, curdian, suberin, lignin, chitin, and the like, as well as combinations thereof.

In specific embodiments, the prebiotic comprises, alternatively consists essentially of, alternatively is, a fiber and/or a starch. In such embodiments, the fiber and/or a starch is not limited, and may be any exemplified by the general and specific examples of fibers and starch herein. In certain embodiments, the prebiotic comprises, alternatively consists essentially of, alternatively is, a resistant starch, i.e., a starch or starch digestion product that is not digested and/or absorbed in the stomach or small intestine of the subject, but instead is adapted to pass to the large intestine of the subject (e.g. for consumption, fermentation, and/or metabolism by the subject's gut microbiota). Examples of suitable resistant starches for use in or as the prebiotic, especially in embodiments where the subject is a mammal such as a human, typically include those categorized by one of skill in the art as category I resistant starch (e.g. starches that are physically inaccessible or indigestible by the subject, such as starches found in seeds, legumes, and unprocessed whole grains), category II resistant starch (e.g. starches resistant to enzymatic degradation in the subject's gut, including those that are enzymatically inaccessible due the conformation of the starch, such high amylose corn starch), category III resistant starch (e.g. starches formed when starch-containing foods such as pasta are cooked and cooled), and/or category IV resistant starch (e.g. starches chemically modified to resist digestion).

It will be appreciated that, with regard to the various forms of the functional composition herein, the prebiotics and/or probiotics above will typically be included in formulations intended for consumption, and less likely those for topical administration.

### Carrier Vehicle

Typically, the functional composition comprises a vehicle (i.e., a carrier vehicle, carrier, etc.) in addition to the cannabis composition. However, it is to be appreciated that, where the cannabis composition itself includes a carrier vehicle (e.g. a solvent) as described above, the cannabis composition may be used directly as the functional composition (i.e., the functional composition may consist of, alternatively may consist essentially of, the cannabis composition). In such instances, the functional composition may comprise a cosolvent or other carrier, or may instead itself be free from, alternatively substantially free from a carrier vehicle other than any provided as part of the cannabis extract itself. In this fashion, it will be appreciated that the functional composition may be prepared in a solid or liquid form.

In certain embodiments, the functional composition comprises a vehicle independently selected based on a desired formulation, form, and/or end use of the functional composition. Suitable vehicles and vehicle components are well known in the supplement (e.g. nutritional and/or food), cosmetic, and pharmaceutical arts, and include water (e.g. purified, deionized, etc.); organic solvents such as alcohols, glycols (e.g. propylene glycol, pentylene glycol, butylene glycol, glycerol/glycerin, etc.), aliphatic alcohols (e.g. lanolin); mixtures of water and organic solvents (such as water and alcohol), and mixtures of organic solvents such as alcohol and glycerol (optionally also with water); lipid-based materials such as fatty acids, acylglycerols (e.g. oils, such as mineral oil, and fats of natural or synthetic origin), phosphoglycerides, triglycerides, sphingolipids, and waxes; protein-based materials such as collagen and gelatin; silicone-based materials (both non-volatile and volatile) such as cyclomethicone, dimethiconol, and dimethicone copolyol; hydrocarbon-based materials such as petrolatum, hydrogenated polyisobutene, and squalane; emollient esters (such as diisobutyl adipate and caprylates), thickening agents (acrylates (carbomers), acrylamides, acryl taurates, hydroxyethylcellulose, methyl cellulose, xanthan gum, pectin, etc.), and the like, as well as derivatives, modifications, and combinations thereof. In some embodiments, the functional composition a cosmetically or pharmaceutically acceptable vehicle, such as saline, buffered saline, 5% dextrose in water, borate-buffered saline containing trace metals, and the like, any of those described herein, and combinations thereof.

As will be appreciated from the description herein, the cannabis composition and the carrier are cooperatively selected based on a desired form and/or end-use of the functional composition. For example, as set forth further below, in certain embodiments, the functional composition is in the form of a supplement (e.g. an oral and/or dietary supplement) and, in other embodiments, the functional composition is in the form of a personal care product (e.g. a topical preparation). These forms of the functional composition may comprise the same or different carrier(s), as well as mixtures thereof.

It is to be appreciated that each additive may be utilized in the functional composition in any amount, which is typically selected based on the type of additive, the formulation of the functional composition, a desired end use of the functional composition, etc. It is also to be appreciated that certain additives may be classified under different terms of art, and may have similar, overlapping, or different functions with additives having different classifications. In certain embodiments, each additive is present in the functional composition in an amount of from greater than 0 to 75 wt. %, based on the total mass of the composition, such as in an amount of 1, 2, 3, 4, 5, 10, 15, 20, 25, 30, 35, 40, 45, 50, 55, 60, 65, 70, or 75 wt. %, based on the total mass of the composition, or in an amount in a range bounded by any two of such values.

Certain embodiments of the functional composition, which vary in terms of formulation and/or form, are described below. However, as introduced above, the functional composition is not particularly limited with regard to substance, form, number of functions, etc. and may comprise any number of components/ingredients in addition to the cannabis composition (or extract), such as the active agents and/or additives described above. In general, the components of functional composition will be individually or collectively selected based on an intended use and/or form of the composition, as will be readily understood by those of skill in the art.

Typically, the functional composition is formulated or otherwise adapted for administration to a mammalian subject (e.g. a human). For example, in various embodiments, the functional composition is adapted to be consumed and/or orally administered to a human subject. As such, the particular additives, carriers, adjuvants, fillers, etc. present in or combined with the functional composition may vary. Moreover, the physical form of the functional composition is not limited, and will be selected based on the particular components of the functional composition, a desired use of the functional composition, etc. As such, as will be understood in view of the description herein, the functional composition may be formulated as a liquid, dry powder, suspension, emulsion, gel, paste, etc., and combinations thereof. In certain embodiments, the functional composition is formulated as, for example, a sterile, non-pyrogenic liquid solution or suspension, a coated capsule, a suppository, a lyophilized powder, a transdermal patch, a softgel, a gummy or other forms known in the art (e.g. tincture, shot, drink, candy bar, bark, chewable, hard candy, etc.). Other examples of suitable forms include solids, gels, liquids, creams, lotions, pomades, mousses, powders, foams, sprays, ointments, or other such preparations where the cannabis composition is disposed in an appropriate carrier vehicle, such as any of those described herein.

The functional composition may be presented in a unit dosage form, such as in a pack (e.g. metal or plastic foil, blister pack, etc.) or dispenser device including one or more unit doses of the composition. The pack or dispenser device may be accompanied by instructions for administration. Useful dosage forms can be prepared by methods and techniques that will be well understood by those of skill in the art and may include the use of additional ingredients in producing tablets, capsules, or liquid dosage forms, such as those described below. Although exemplary dosages, dose frequencies, and methods of administration are discussed herein, these are merely exemplary and it is to be understood that the dose, dose frequency, and mode of administration may vary according to the age, body weight, condition and response of the individual subject, consumer, or patient, and the particular formulation of the functional composition.

As will be understood in view of the description below, the functional composition, in any form, may be administered as needed, daily, several times per day or in any suitable regimen such that a desired outcome is achieved. In a treatment method utilizing the functional composition, the frequency of administration can depend on several factors, including a desired level of prevention or amelioration. Generally, an exemplary regimen includes administration of the functional composition to the subject once or twice daily, e.g. including an administration in the morning and/or an administration in the evening. The amount of the composition administered to the subject during each administration (i.e., the dose) may depend on several factors, such as the level of results desired, and the specific composition being utilized, the number of doses being administered, etc. In general, the functional composition is administered in a therapeutically or physiologically effective amount, which, as introduced above, relates to an amount (i.e., a quantity) of a composition (e.g. the functional composition of the present embodiments) required to achieve a particular therapeutic and/or prophylactic effect, such as in treating a subject (e.g. by ameliorating a condition thereof). Likewise, as used herein, the term “physiologically effective amount” relates to an amount of a composition (e.g. the functional composition of the present embodiments) required to achieve a desired physiological effect. Such effective amounts are typically measured and/or expressed in terms of the amount of the functional composition over time (e.g. g/day, mg/day, etc.), but may also incorporate the body weight of the subject (e.g. in kg), as expressed by the unit g/kg/day. Typically, the functional composition is administered in an amount effective to provide the cannabis extract (i.e., the cannabis phytocompounds thereof) to the subject. In certain embodiments, the functional composition is administered in an amount effective to ameliorate a condition of the subject. In these or other embodiments, the functional composition is administered in an amount effective to ameliorate at least two conditions of the subject, including any of those described herein.

### Consumable Composition/Supplement

As introduced above, in certain embodiments, the functional composition is formulated as a consumable composition. The particular formulation of the consumable composition is not particularly limited, and may comprise various ingredients and/or components as described below. In general, the consumable composition comprises the cannabis composition and one or more other components, which may generally be selected from carriers, coactives, additives, excipients, etc.

### Forms of Consumable Composition

The particular form of the consumable composition for oral administration is not particularly limited. For example, in certain embodiments, the consumable composition may be presented in discrete units (e.g. capsules, cachets, lozenges, tablets, etc.) that each containing a predetermined amount of the consumable composition (e.g. a recommended dose). However, the consumable composition may compose any consumable form, such as a dry powder, a solution, a suspension, an emulsion, or the like. In certain embodiments, the consumable composition is a dry powder. In some embodiments, the consumable composition is adapted to be consumed as a liquid. For example, the consumable composition may be a dry powder that is adapted to be combined with a consumable liquid (e.g. water) to form a consumable liquid solution, suspension, or emulsion comprising the consumable composition.

In certain embodiments, the consumable composition may be adapted to be mixed with a foodstuff or beverage. The term “foodstuff” is used herein to refer to a material that may be used as a food. As such, in certain instances the term foodstuff is used to describe a composition that may be consumed (e.g. by eating) by a living organism (e.g. a mammal), such as for nourishment and/or sustenance. Likewise, the term “beverage” as used herein refers to a potable liquid or other non-solid composition. As such, in certain instances the term beverage is used to describe a non-solid (e.g. liquid, slurry, suspension, etc.) composition that may be consumed by a living organism for nourishment and/or sustenance. As such, in particular instances the terms “beverage” and “foodstuff” may overlap. In certain instances, the term “nutritional composition” is used to describe a foodstuff and/or beverage formulation that can be eaten or drunk by a human subject for nutrition. Accordingly, in some embodiments, the consumable composition is, alternatively is a component of, a foodstuff or beverage.

### Food Additive

In these or other embodiments, the consumable composition may be further defined as a food additive. As used herein, the term “food additive” refers to an ingredient, additive, component, or supplement suitable for incorporation in a foodstuff and/or beverage to confer a technical, nutritional, and/or health benefit (i.e., a function) to a host that consumes the foodstuff and/or beverage. Accordingly, such benefits may be closely related to the presence of the cannabis extract being administered to the subject. The food additive can be added to different types of food including, but not limited to, medical foods, dietetic foods, and supplements. Certain aspects of the present embodiments can include the use of the consumable composition as a food additive, and the use of the consumable composition in methods of preparing foodstuffs and/or beverages.

In general, when utilized as a component of a foodstuff or beverage, the foodstuff or beverage comprises an admixture of the consumable composition with one or more feed products, liquids, supplements, or combinations thereof. However, in certain embodiments, the consumable composition may itself be further defined as a foodstuff or beverage composition, depending on the quantity, nature, and identity of individual additives and components present in the consumable composition, such as those described above. Thus, it is to be appreciated that the embodiments described herein with respect to the consumable composition are intended to equally encompass the foodstuff or beverage, a food or beverage product, and/or a food supplement comprising the consumable composition. Accordingly, any amounts and/or examples of such components described herein with respect to the consumable composition itself may equally apply to the foodstuff or beverage comprising the consumable composition.

### Nutritional Composition

In some embodiments, the foodstuff or beverage comprising the consumable composition is further defined as a nutritional composition. In these or other embodiments, the nutritional composition is in the form of a dry food concentrate, which may be mixed with liquid or food and subsequently consumed. It is to be appreciated that the nutritional composition is distinguished from a vaccine, and the consumable compositions described herein may be free, alternatively substantially free, from a vaccine.

### Nutritional Supplement

In certain embodiments, the consumable composition may be further defined as a nutritional supplement, or as a complete nutritive. As used herein, the term “supplement” relates to a nutritional supplement which is a concentrated source of nutrient or alternatively other substances with a nutritional or physiological effect whose purpose is to supplement the normal diet. For example, the consumable composition may be formulated to provide a mammal (e.g. a human), via consumption of the consumable composition, with at least 5%, alternatively at least 10%, alternatively at least 25%, alternatively at least 50%, alternatively at least 75%, alternatively at least 90%, of daily calories required by the mammal. However, it is to be appreciated that a daily calorie requirement is dependent on several factors, including the gender, height, and/or age of the mammal, and thus the percentage of caloric requirement provided by the consumable composition will be dependent on the particular person consuming the nutritional composition. For example, a 30 year old human male of 80 kg body weight and 180 cm height has a daily calorie requirement of around 2900 cal (calories) to maintain his body weight whereas a 30 year old human female of 55 kg body weight and 165 cm height has a daily calorie requirement of around 2100 cal to maintain her body weight.

### Medical Food

In some embodiments, the foodstuff or beverage is further defined as a medical food. As such, it is to be appreciated that the medical food comprises the consumable composition, and may be the same as or different from the nutritional composition described above. As used herein, the term “medical food” is typically used to refer to a food for a special dietary use, such as a food formulated for dietary management of a medical condition (e.g. based upon scientific or medical evaluation). However, it is to be appreciated that the term “medical food” may have one or more particular definitions depending on, for example, geographic location, specific use, regulatory agency, and the like. For example, in certain cases, the term medical food may be defined as a food which is formulated to be consumed or administered enterally under the supervision of a physician and which is intended for the specific dietary management of a disease or condition for which distinctive nutritional requirements, based on recognized scientific principles, are established by medical evaluation (see, e.g. section 5(b) of the Orphan Drug Act (21 U.S.C. 360ee (b) (3)), which is incorporated herein by reference). In these or other instances, the term medical food may be defined as a food for special dietary use as a food that has been specially processed or formulated to meet the particular requirements of a person: (a) in whom a physical or physiological condition exists as a result of a disease, disorder, or injury; or (b) for whom a particular effect, including but not limited to weight loss, is to be obtained by a controlled intake of food (see, e.g. section 8.24.001 of the Canadian Food and Drug Regulations (FDR, C.R.C., c. 870) (as amended 13 Jun. 2017)), which is incorporated herein by reference).

### Animal Food

In certain embodiments, the consumable composition is further defined as an animal food. In such embodiments, the consumable composition is typically formulated for ingestion by one or more non-human animals, such as livestock including cattle, swine, horses, sheep, goats, poultry, and fish, domesticated companionship species such as dogs, cats, fish, and rodents, undomesticated wildlife such as deer, moose, elk, migratory, and non-migratory fowl, those non-human animals described herein, and combinations thereof. In certain instances, administering the consumable composition as the animal food to a non-human subject (e.g. an animal) may result in an increased yield in one or more commodities produced by the host, such as eggs, meat, milk, wool, etc.

### Dosage Forms

In certain embodiments, the consumable composition is provided in a dosage form, such as a consumable dosage form or a pharmaceutical dosage form. In such embodiments, the consumable composition is typically adapted specifically for oral administration (i.e., being taken by mouth), rather than for enteral administration, which encompasses the same oral administration routes as well as buccal, sublabial, and sublingual administrations. However, it will be appreciated from the examples below that certain dosage forms may be suitable for multiple routes of enteral administration. Those of skill in the art will appreciate that the description of the dosage forms herein may be read upon the functional composition itself instead of the consumable composition prepared therefrom. Specifically, in certain arts, it will be understood that pharmaceutical and other dosage forms adapted for enteral or, more specifically, oral administration, may not fall within accepted nomenclature or regulatory guidelines concerning “consumables”. As such, it is to be understood that the consumable dosage forms and pharmaceutical dosage forms may differ from one another in terms of particular contents, components, dimensions, parameters, and materials utilized.

As used herein, the term “consumable dosage form” refers to an end-use product in a form marketed for use. For example, a consumable dosage form of the consumable composition typically includes combination of i) a mixture of the consumable composition and nonactive components (e.g. excipients), and ii) any non-reusable packaging (e.g. capsule shells, etc.). In this fashion, the combination is typically sized to provide a single dose, which may be independently selected.

It will be appreciated that the similar term “pharmaceutical dosage form” may be used in a similar fashion with the ordinary and customary meaning in the art, i.e., to refer to a form of the consumable composition (or the functional composition) prepared as a pharmaceutical product in an end-use form (e.g. a mixture of the functional composition, any active pharmaceutical or drug components, any excipients, and any non-reusable materials). Those of skill in the art will also appreciate that the term “pharmaceutical dosage form” can also be used to refer to but only the chemical formulation of constituent drug substance(s) of such a product, as well as any blends thereof, e.g. without consideration as to ultimate configuration as pill, capsule, patch, etc.

Dosage forms suitable for the consumable composition are typically limited to oral dosage forms, and generally include liquids, solids, and semi-solid dosage forms without limitation. Examples of these particular forms include pills, tablets, orally dissolving tablets (ODTs), capsules, drinks, syrups, tinctures, etc., which may be selected in view of a particular formulation and/or desired route of administration. As such, it is to be appreciated that various dosage forms may be utilized for any given formulation of the consumable composition, with such forms typically selected based on a condition to be treated. Other dosage forms may also be utilized, and may encompass and/or include different forms of the consumable composition therein (e.g. solution, suspension, emulsion, powder, etc.). For examples, in some embodiments, the consumable composition is formulated and prepared as a candy, such as a gummy, lollipop, lozenge or other hard candy, candy bar, bark, or chewing gum.

It will be appreciated that certain dosage forms may be adapted for general enteral administration or, alternatively, for specific oral administration. For example, dosage forms such as tablets may be formulated to swallow, chew, dissolve in water, or dissolve under the tongue. Likewise, capsules and chewable capsules each may optionally comprise a coating adapted to dissolve in the stomach, bowel, etc. for a targeted release of the composition in such organs. Such coatings and similar features are generally referred to as “time-release,” “delayed-release,” and/or “sustained-release”.

### Active Amount/Concentration

The consumable composition, in any of the forms described herein, may comprise any amount of the cannabis extract, which will typically be limited by nature of the particular cannabis phytocompound content therein. Specifically, daily intake limits are specified by numerous regulatory/governing organizations, and are being regularly investigated and updated based on the increasing understanding of biological effects of such compounds. As such, particular amounts and limits of any given cannabis phytocompound present in the consumable composition may vary, even irrespective of potency (i.e., where a more-potent content is prohibited by law or rule). As but one example, the consumable composition may be formulated to comprise up to 150 mg of CBD per daily dose, e.g. in a single serving (i.e., a “single daily dose”) or split across multiple servings to be ingested in a 24 hour period. In some embodiments, the consumable composition may be formulated to comprise up to 100 mg of CBD per daily dose.

In some embodiments, the consumable composition is free from, alternatively substantially free from, a CBD isolate. For example, in certain such embodiments the cannabis extract of the consumable composition comprises a Cannabis sativa flower or stalk extract comprising a CBD content of less than 90%, alternatively less than 85%, alternatively less than 80%, by weight. Such limitations with respect to CBD in the preceding description may apply to any given cannabis phytocompound, the amount and contents of which will be independently selected by those of skill in the art. Particular amounts and examples of such contents are described and exemplified herein.

### Multi-Component Compositions

In particular embodiments, the consumable composition is formulated with more than one different types of cannabis compositions, which may each be independently selected from the cannabis extracts described herein as well as other components comprising a cannabis phytocompound. Examples of such consumable compositions may thus include those extract combinations described above. For example, in certain embodiments, the consumable composition comprises a combination of cannabis powder, cannabis juice or juice concentrate, and an aqueous/alcohol-based cannabis extract composition. In specific embodiments, the consumable composition comprises a combination of hemp powder, hemp juice or juice concentrate, and an aqueous/alcohol-based hemp extract composition. In these embodiments, the hemp powder may act as a drying agent or carrier. In these or other embodiments, the combination of cannabis extracts may comprise a separate carrier. Examples of such carriers are described herein, and are exemplified by microcrystalline cellulose, dicalcium phosphate, maltodextrin, croscarmellose sodium, silicon di-oxide, acacia, corn starch, hydroxypropyl methylcellulose, modified food starch, magnesium stearate, sugarcane fiber, sucrose, sodium alginate, pea starch, corn starch, soy protein isolate, carnauba wax, soybean oil, etc.

In various embodiment, the cannabis (or consumable) composition is a multi-hemp extract composition. The multi-hemp extract composition comprises a first hemp component comprising a hemp dehydrate, a second hemp component different from the first hemp component, the second hemp component comprising a hemp juice, a third hemp component different from the first and second hemp components, the third hemp component comprising a hemp extract, and a carrier component different from each of the hemp components.

The hemp dehydrate is generally as described above for the cannabis flour. Thus, the hemp dehydrate can be in the form of a powder or flour. In various embodiments, the hemp dehydrate is obtained from at least one of Cannabis leafs, buds, and flowers. In certain embodiments, the hemp dehydrate is prepared from Cannabis buds.

The hemp dehydrate can be used in various amounts. In certain embodiments, the hemp dehydrate is present in an amount of from about 15 to about 50 wt. %, optionally about 20 to about 45 wt. %, optionally about 25 to about 40 wt. %, optionally about 25 to about 35 wt. %, or optionally about 25 to about 30 wt. %, each based on a (total) dry-weight basis of the composition.

The hemp juice is generally as described above for the cannabis juice. For example, the hemp juice can be in the form of a liquid concentrate. Such concentrates can have various % solids, such as at least 20% solids. In various embodiments, the hemp juice is obtained from aerial portions of immature Cannabis plants.

The hemp juice can be used in various amounts. In certain embodiments, the hemp juice (based on a total solids content thereof) is present in an amount of from about 15 to about 50 wt. %, optionally about 20 to about 45 wt. %, optionally about 25 to about 40 wt. %, optionally about 30 to about 40 wt. %, or optionally about 30 to about 35 wt. %, each based on a (total) dry-weight basis of the composition.

The hemp extract is generally as described above. For example, the hemp extract can be a solvent (e.g. alcohol) extract of hemp. In various embodiments, the hemp extract is obtained from solvent extraction of at least one of Cannabis buds and flowers. The hemp extract can be in a carrier or vehicle, such as MCT oil.

The hemp extract can be used in various amounts. In certain embodiments, the hemp extract (based on a total solids or non-aqueous content thereof) is present in an amount of from about 5 to about 30 wt. %, optionally about 5 to about 25 wt. %, optionally about 5 to about 20 wt. %, optionally about 7.5 to about 17.5 wt. %, or optionally about 7.5 to about 15 wt. %, each based on a (total) dry-weight basis of the composition.

The carrier component is generally as described above for the additive component. The carrier component may also be referred to as an excipient component. In various embodiments, the carrier component comprises silicon dioxide (SiO₂). In further or other embodiments, the carrier component comprises (or further comprises) at least one selected from the group consisting of sunflower lecithin, maltodextrin, pectin, gum arabic, tara gum, xanthan gum, pectin, corn starch, tapioca starch, pea starch, oyster shell powder, tricalcium phosphate, sugar, and combinations thereof.

In specific embodiments, the carrier component is not naturally occurring. In other words, the carrier component is not a product of nature in these specific embodiments.

The carrier component can be used in various amounts. In certain embodiments, the carrier component is present in an amount of from about 10 to about 50 wt. %, optionally about 15 to about 45 wt. %, optionally about 20 to about 40 wt. %, optionally about 25 to about 35 wt. %, or optionally about 27.5 to about 32.5 wt. %, each based on a (total) dry-weight basis of the composition.

In many embodiments, the multi-hemp extract composition is further defined as an oral composition. The oral composition is formulated for oral administration to a subject. In such embodiments, the multi-hemp extract composition may also be referred to as an ingestible composition (or consumable composition). The oral composition can be in various forms, as described herein, e.g. tablets, pills, etc.

The multi-hemp extract composition can be in various forms, such as liquids, powders, etc. In various embodiments, the multi-hemp extract composition is in the form of a powder. In various embodiments, the powder has a moisture content less than 5 wt. %, optionally less than 4 wt. %, or approaching 0 wt. %. The (dry) powder can be obtained by using dry components, or by subsequently drying the multi-hemp extract composition. Various drying techniques are understood in the art. In certain embodiments, the multi-hemp extract composition is dried with a refractive (or refractance) window dryer (RWD). Drying methodologies utilizing RWDs are understood in the art, and one of skill in the art can readily determine drying temperatures, times, etc.

The multi-hemp extract composition can used to administer at least one cannabis phytocompound to a subject. In various embodiments, the multi-hemp extract composition is orally administered to the subject. Such methods, subjects, and routes of administration are as described herein.

### Topical Composition

As introduced above, in certain embodiments, the functional composition is formulated as the topical composition. In general, the topical composition comprises the cannabis composition and one or more other components, which may generally be selected from carriers, coactives, additives, excipients, etc. It is believed that the topical composition has improved characteristics and utility owing to the particular forms and components thereof. The topical composition can be used in ameliorating a skin-related condition of a subject, e.g. by reducing or preventing chronic and/or acute inflammation, improving epidermal barrier function, etc. in the skin of the subject, upon administration of the topical composition thereto. Moreover, the topical composition can be used to administer the cannabis phytocompounds of the cannabis extract to ameliorate other conditions of a subject, including those described above.

In general, the topical composition is formulated or otherwise adapted for topical administration. More specifically, the topical composition can be generally formulated to provide the cannabis extract to a part of a subject via direct application (e.g. topically to surfaces such as skin, mucous membranes, etc.). As such, the topical composition optionally is formulated or otherwise adapted for topical administration to a mammal (e.g. a human). For example, in various embodiments, the topical composition can be formulated to be administered to the skin of a human.

Certain embodiments of the topical composition, which vary in terms of formulation and/or form, are described below. However, as introduced above, the topical composition is not particularly limited with regard to substance and/or form, and may comprise any number of components/ingredients in addition to the cannabis composition, such as the other active agents and/or additives described herein. Likewise, the particular additives, carriers, adjuvants, fillers, etc. present in or combined with the topical composition may also vary. In general, the components of the topical composition will be individually or collectively selected based on an intended use of the topical composition. Moreover, the amount of any particular component will be individually selected, e.g. based on a desired end form (e.g. cream vs. spray, etc.). As will be appreciated by those of skill in the art in view of the description and examples herein, however, the particular components will typically be selected to maximize the effectiveness of the cannabis composition, e.g. by avoiding components that will inhibit and/or prevent migration of the components thereof (e.g. cannabis phytocompounds) to the skin surface, and instead selecting carrier vehicles that will facilitate transport of the cannabis phytocompounds to the skin surface or through the skin, as desired.

The topical composition may comprise any form for topical application, including powders, sprays, ointments, pastes, creams, lotions, gels, solutions, and the like, as well as combinations thereof. Said differently, the physical form of the topical composition is not particularly limited. Rather, the topical composition may be formulated as a liquid, dry powder, suspension, dispersion, emulsion (e.g. oil-in-water, water-in-oil, water-in-silicone, etc.), gel, paste, etc., and combinations thereof. As such, it will be appreciated that the topical composition may be provided in the form of a gel, a cream, an aerosol spray, a foam, a liquid, a mousse, a pomade, a powder, a solid, or an ointment. In some embodiments, the topical composition is provided as an aqueous solution, dispersion, or emulsion.

As described above, the topical composition may be utilized to ameliorate a skin condition of and/or confer a skin-related health benefit to a subject. As such, in certain embodiments, the topical composition can comprise an active agent in addition to the mucilage plant extract, which may provide benefits that are the same as, similar to, or different from the benefits of the mucilage plant extract. For example, in various embodiments, the topical composition comprises multiple active agents in addition to the cannabis composition, which can each be independently selected (e.g. based on a desired property of the active agent, such as a benefit conferred to the subject via application of the topical composition). Such active agents may include antibiotics, probiotics, prebiotics, postbiotics, parabiotics, pharmaceuticals, nutraceuticals, anesthetics, counterirritants, chondroprotective agents, etc., and are exemplified by those listed herein.

In some embodiments, the topical composition can comprise additives selected specifically for use in formulating and/or using the topical composition, such as a pharmaceutically/medically acceptable carrier, a functional additive, a formulation additive, or combinations of such additives, e.g. selected based on a desired form of the topical composition, use of the topical composition, etc.

In some embodiments, the topical composition comprises a pharmaceutically acceptable carrier. The pharmaceutically acceptable carrier may be a non-toxic carrier, a physiologically acceptable carrier, etc. The pharmaceutically acceptable carrier may be in the form of an emulsion, a paste, a cream, a lotion, a gel, jelly, an ointment, an oil, an aerosol, a powder, a solvent, a liposome, a micelle, a peptide (e.g. albumin), a synthetic polymer (e.g. polyethylene glycol), a natural polymer (e.g., hyaluronic acid, dextran, chitosan), an n-dimensional material (e.g. where n=0, 1, 2, 3) such as, for example, a 0-dimensional nanomaterial (e.g. quantum dot, nanoparticle, etc.), a I-dimensional nanomaterial (e.g. a nanotube, a nanorod, etc.), a 2-dimensional nanomaterial (e.g. a quantum well, a film, etc.), a 3-dimensional material, such as a matrix (e.g. polymeric matrix such as polyethylene glycol (PEG)), etc. The pharmaceutically acceptable carrier may provide timed release, modulate the pharmacokinetic properties (e.g., absorption, distribution, metabolism, excretion) of CBD or any other cannabis phytocompounds of the cannabis composition, modulate the pharmacodynamic properties (e.g., concentration at a site of action, resulting effect, etc.) of such cannabis phytocompounds, etc. For example, the pharmaceutically acceptable carrier may be an active carrier facilitates transport of CBD or any other cannabis phytocompounds of the cannabis composition across skin. Examples of such active carriers are described below.

The carrier optionally can be selected to be generally compatible with the individual components of the topical composition and to enhance, or to not interfere significantly with, the application of the cannabis composition, or at least the cannabis phytocompounds thereof, to a surface of the skin of a subject and, optionally, to enhance or not interfere with transport of other components of the topical composition to or through the skin. General examples of suitable carriers include those that promote and/or facilitate transport of various components of the topical composition through skin. Particular examples of carriers include water (e.g. deionized), oils and/or waxes (e.g. mineral oils, synthetic oils, natural oils such as jojoba oil, castor oil, etc., and waxes formed therewith), alcohols (e.g. monols, diols, and polyols such as ethanol, isopropanol, butanediol, 1,2,6-hexanetriol etc., glycols such as ethylene glycol, propylene glycol, etc.), polyoxyalkylenes and/or polyoxyalkylene esters (e.g. polyethylene glycols, polypropylene glycols, mixed polyalkylene glycols, polyethylene glycol-8 stearates, etc.), fatty acid esters (e.g. alkyl stearates, oleates, linoleates, isopropyl palmitate, etc.), organic polymers (e.g. polyacrylamides), organic solvents (e.g. dimethylsulfoxide, dimethylformamide, dimethylacetamide, methylsulfonylmethane), and the like, as well as derivatives, modifications, and combinations thereof, and any of the other carriers described herein, such as applicable vehicle and/or vehicle components described above

In some embodiments, the topical composition comprises an active carrier. The active carrier is adapted to enhance the bioavailability of the active components of the topical composition. For example, the active carrier typically assists in penetration of the active components of the topical composition through the pores of the skin. The active carrier may also possess good skin moisturizing activity, reduces emulsion particle sizes, which helps to achieve better stability of the topical composition. In some embodiments, the active carrier improves the aesthetic appeal of the topical composition. In some embodiments, the active carrier works synergistically with additives in the topical composition, such as preservatives. In some embodiments, the active carrier improves the water resistance of the cannabis composition, enhancing the ability of the cannabis composition to be incorporated, e.g. into a sunscreen formulation. In some embodiments, the active carrier promotes skin repair and restructure, acts as an anti-wrinkle agent, exhibits broad-spectrum antimicrobial activity, etc. In some embodiments, the active carrier possesses good solvent and solubility properties.

Non-limiting examples active carriers, as well as components useful in preparing the same, include aqueous solvents and organic solvents, for example, alcohols such as ethanol, propanediol, butylene glycol, isopropanol, glycerin, and mixtures thereof. In some embodiments, the active carrier may include pentylene glycol, ethoxydiglycol, bis-ethoxydiglycol cyclohexane 1,4-dicarboxylate, dipalmitoyl hydroxyproline, potassium palmitoyl hydrolyzed wheat protein, glyceryl stearate, cetearyl alcohol, potassium lauroyl wheat amino acids, palm glycerides, capryloyl glycine, potassium palmitoyl hydrolyzed oat protein, behenyl alcohol, palm glycerides, sodium stearoyl glutamate, sucrose palmitate, polyglyceryl-3 sorbityl linseedate, or combinations thereof. In some embodiments, the active carrier comprises pentylene glycol, ethoxydiglycol, bis-ethoxydiglycol cyclohexane 1,4-dicarboxylate, dipalmitoyl hydroxyproline, potassium palmitoyl hydrolyzed wheat protein, glyceryl stearate, cetearyl alcohol, potassium lauroyl wheat amino acids, palm glycerides, capryloyl glycine, potassium palmitoyl hydrolyzed oat protein, behenyl alcohol, palm glycerides, sodium stearoyl glutamate, sucrose palmitate, polyglyceryl-3 sorbityl linseedate, or a combination thereof. according to some embodiments, the anti-inflammatory agent may include alpha-bisabolol, allantoin, sea whip extract, Chamomilla recutita (matricaria) extract, tocopheryl acetate, Camellia sinensis leaf extract, Curcuma longa (turmeric) root extract, Avena sativa (oat) kernel extract, Magnolia officinalis bark extract, Vitis vinifera (grape) seed extract, Zingiber officinale (ginger) root extract, dipotassium glycyrrhizinate, or a combination thereof. In some embodiments, the collagen synthesis enhancer may include methylglucoside phosphate, inula crithmoide flower/leaf extract, collagen prepeptide (e.g., g-p-hyp tripeptide), madecassoside, asiaticoside, or a combination thereof. The anti-wrinkle agent may include an Echinacea purpurea extract, cichoric acid, resveratrol, trifluoroacetyl tripeptide-2, or a combination thereof. The keratinocyte growth factor stimulant may include a purified form (purity of at least 95%) or an enriched extract of swertiamarin.

In particular embodiments, the topical composition can comprise a functional additive. The functional additive is not limited, and may comprise, optionally may be, any compound or composition selected to provide a functional characteristic to, or impart a function on, the topical composition. Examples of such functional additives include anti-oxidants (e.g. alkylates hydroxytoluenes, hydroxyanisoles, etc., propyl gallate, etc.), colorants, moisturizers and emollients (e.g. sunflower oil, jojoba oil, isopropyl palmitate, etc.), perfumes (e.g. natural perfumants such as rosemary oil, synthetic perfumes, etc.), cooling agents (e.g. peppermint oil), preservatives (e.g. antimicrobial and antifungal agents, such as propylene glycol, methyl paraben, propyl paraben, diazodinyl urea, etc.), and the like, as well as derivatives, modifications, and combinations thereof.

For example, in certain embodiments, the topical composition can comprise a moisturizer. Examples of suitable moisturizers include hydroxy acids (e.g. lactic acid) and their salts, glycerol, propylene glycol, pentylene glycol, butylene glycol, sodium salts of pyrrolidone carbonic acid (i.e., sodium PCA), sodium hyaluronate, polyethylene glycols (PEG) (e.g. CARBOWAX PEG 200, CARBOWAX PEG 400, CARBOWAX PEG 800, etc.), and the like, as well as derivatives, modifications, and combinations thereof. In these or other embodiments, the topical composition comprises an emollient and/or a humectant. Examples of suitable emollients or humectants include cetyl palmitate, glycerol (i.e., glycerin), polypropylene glycol-15 stearyl ether (i.e., PPG-15 stearyl ether), lanolin and derivatives thereof (e.g. lanolin alcohol, etc.), cholesterol, petrolatum, isostearyl neopentanoate, octyl stearate, mineral oil, isocetyl stearate, myristyl myristate, octyl dodecanol, octyl palmitates (e.g. 2-ethylhexyl palmitate), dimethicone, phenyl trimethicone, cyclomethicone, C₁₂-C₁₅ alkyl benzoates, dimethiconol, propylene glycols, pentylene glycols, Theobroma grandiflorum seed butter, shea butter, ceramides (e.g. ceramide 2, ceramide 3, etc.), hydroxypropyl bispalmitamide MEA, hydroxypropyl bislauramide MEA, hydroxypropyl bisisostearamide MEA, 1,3-bis-(N-2-(hydroxyethyl)stearoylamino)-2-hydroxy propane, bis-hydroxyethyl tocopherylsuccinoylamido hydroxypropane, urea, aloe, allantoin, glycyrrhetinic acid, dicaprylate/dicaprates, and the like, as well as derivatives, modifications, and combinations thereof.

In certain embodiments, the topical composition can comprise a preservative. Examples of suitable preservatives include ureas (e.g. imidazolidinyl urea, diazolidinyl urea, etc.), phenoxyethanol, sodium methyl paraben, methylparaben, ethylparaben, propylparaben, potassium sorbate, sodium benzoate, sorbic acid, benzoic acid, formaldehyde, citric acid, sodium citrate, chlorine dioxide, quaternary ammonium preservative compounds (e.g. benzalkonium chloride, benzethonium chloride, cetrimide, dequalinium chloride, cetylpyridinium chloride, etc.), mercurial preservative agents (e.g. phenylmercuric nitrate, phenylmercuric acetate, thimerosal, etc.), piroctone olamine, Vitis vinifera seed oil, alcoholic preservative agents (e.g. chlorobutanol, dichlorobenzyl alcohol, phenylethyl alcohol, benzyl alcohol, etc.), and the like, as well as derivatives, modifications, and combinations thereof. In these or other embodiments, the topical composition comprises an antioxidant. Examples of suitable antioxidants include ascorbic acid and esters thereof, sodium bisulfite, butylated hydroxytoluene, butylated hydroxyanisole, tocopherols (e.g. α-tocopherol), tocopheryl acetate, sodium ascorbate/ascorbic acid, ascorbyl palmitate, ascorbyl glucoside, propyl gallate, chelating antioxidants (e.g. ethylenediaminetetraacetic acid (EDTA), disodium EDTA, etc.), citric acid, sodium citrate, and the like, as well as derivatives, modifications, and combinations thereof.

In certain embodiments, the topical composition can comprise a formulation additive. The formulation additive is not limited, and may comprise, optionally may be, any compound or composition selected to impart a physical characteristic to the topical composition. Examples of such formulation additives include emulsifiers (e.g. isoparaffins such as C₁₃-C₁₄ isoparaffin, surfactants such as laureth-7, polymers such as polyacrylamides and polyalkyleneglycols, etc.), buffers, excipients, propellants, and the like, and combinations thereof. Typically, the formulation additive is selected based on the desired form of the topical composition. For example, in some embodiments, the topical composition is formulated as an ointment, paste, cream, and/or gel, and comprises an excipient exemplified by animal and vegetable fats, oils, waxes, paraffins, starch, tragacanth, cellulose derivatives, polyethylene glycols, silicones, bentonites, silicic acid, talc, zinc oxide, and the like, as well as derivatives, modifications, and combinations thereof. In certain embodiments, the topical composition is formulated as a powder and/or spray, and comprises an excipient exemplified by lactose, talc, silicic acid, aluminum hydroxide, calcium silicates, polyamide powders, and the like, as well as derivatives, modifications, and combinations thereof. In particular embodiments, the topical composition is formulated as a spray and a propellant, such as a volatile organic compound exemplified by halogenated hydrocarbons (e.g. hydrocarbons substituted with chlorine, fluorine, or both) and low molecular weight unsubstituted hydrocarbons (e.g. butane, propane, etc.). In general, when present, the topical composition comprises the formulation additive in an amount of from 1 to 50, alternatively from 1 to 20 wt. %, based on the total weight of the topical composition.

In particular embodiments, the topical composition can comprise a lipophilic solubilizer. Some examples of lipophilic solubilizers include non-comedogenic esters, such as adipates (e.g. diisobutyl adipate), caprylates, isononanoates (e.g. isononyl neopentanoate), ethoxylated triglycerides, and the like, as well as modifications, derivatives, and combinations thereof. Other examples of lipophilic solubilizers generally include cetyl esters, polyethylene glycol cetyl esters, hydrogenated polyisobutenes, argan oil, soybean oil, chemical UV filters/boosters (e.g. octisalate, octinoxate, butyl octyl salicylate, etc.), and the like, as well as modifications, derivatives, and combinations thereof.

In some embodiments, the topical composition can comprise a free radical stabilizer. Examples of free radical stabilizers generally include lipophilic antioxidants, such as tocotrienols, carotenoids (e.g. tocopherol, tocopherol acetate, retinyl palmitate, tetrahexydecyl ascorbate, lutein, natural oils rich in unsaturated fatty acids such as docosahexaenoic acid, etc.), and the like, as well as modifications, derivatives, and combinations thereof

In certain embodiments, the topical composition can comprise a surfactant. Examples of suitable surfactants include ionic (e.g. anionic, zwitterionic, etc.) and non-ionic surfactants. Some specific examples of such surfactants include polysorbates (e.g. polyoxyethylene (20) sorbitan monolaurate (i.e., Polysorbate 20), polyoxyethylene (20) sorbitan monopalmitate (i.e., Polysorbate 40), polyoxyethylene (20) sorbitan monostearate (i.e., Polysorbate 60), polyoxyethylene (20) sorbitan monooleate (i.e., Polysorbate 80), etc.), vegetable sorbitan stearates, steareth-10 and other octadecyl polyoxyethylene ethers, sodium dodecyl sulfates (e.g. sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (e.g. sodium deoxycholate, sodium cholate, etc.), polyoxyl castor oil, nonylphenol ethoxylate, cyclodextrins, lecithin, dimethicone copolyol, lauramide diethanolamine, cocamide diethanolamine, cocamide monoethanolamine, betaines (e.g. oleyl betaine, cocamidopropyl betaine, etc.), cocamidopropyl phosphatidyl PG-dimonium chloride, dicetyl phosphate (dihexadecyl phosphate), ceteareth-10 phosphate, polyglyceryl-2 triisostearate, cetyl PEG/PPG-1/1 dimethicone (ethoxylated or organo-modified silicones for W-in-Si emulsions, glyceryl stearate, glyceryl dilaurate, lecithin, unsaturated lecithin, etc.), methylbenzethonium chloride, and the like, as well as modifications, derivatives, and combinations thereof.

In some embodiments, the topical composition can comprise an emulsifier, which may be the same as or different from the surfactant. Examples of such emulsifiers include behentrimonium methosulfate-cetearyl alcohol, non-ionic emulsifiers (e.g. emulsifying waxes), polyoxyethylene oleyl ethers, polyethylene glycol stearates (i.e., PEG-40 stearate, PEG-100 stearate, etc.), cetostearyl alcohols (e.g. cetearyl alcohol), ceteareth-12, ceteareth-20, ceteareth-30, ceteareth alcohol, glyceryl stearate, steareth-2 and steareth-20, cationic emulsifiers (e.g. stearamidopropyl dimethylamine, behentrimonium methosulfate, etc.), and the like, as well as modifications, derivatives, and combinations thereof.

In particular embodiments, the topical composition can comprise a viscosity adjusting agent (e.g. a thickening or thinning agent, which may be referred to as a viscosity modifier). Examples of such agents generally include protective colloids, non-ionic gums such as hydroxyethylcellulose, xanthan gum, and sclerotium gum, magnesium aluminum silicate, silica, microcrystalline waxes, beeswax, paraffin, cetyl palmitate, and the like, as well as modifications, derivatives, and combinations thereof.

In certain embodiments, the topical composition can comprise one or more additional components, which may comprise or be selected skin protectants, adsorbents, demulcents, emollients, moisturizers, hydrators, buffering agents, sustained release materials, solubilizing agents, skin-penetration agents, skin soothing agents, deodorant agents, antiperspirants, sun screening agents, sunless tanning agents, vitamins, hair conditioning agents, anti-irritants, anti-aging agents, abrasives, absorbents, anti-caking agents, anti-static agents, astringents (e.g. witch hazel, alcohol, chamomile extract, etc.), binders/excipients, buffering agents, chelating agents, film forming agents, conditioning agents, opacifying agents, lipids, pH adjusters (e.g. citric acid, sodium hydroxide, sodium phosphate monobasic, sodium phosphate dibasic, etc.), and the like, as well as modifications, derivatives, and combinations thereof. Specific examples of such additional components are exemplified in U.S. Patent Application Publication No. 2018/0110722 A1, the disclosure of which regarding topical composition components is incorporated by reference herein.

In some embodiments, the topical composition comprises one or more additional components selected from thickeners, emulsion stabilizers, emulsifiers, emollients, conditioners, humectants, moisturizers, preservatives, antioxidants, pH adjusters, surfactants, fragrances, etc. In some such embodiments, the topical composition may further include at least one additional cosmetic agent, such as a vitamin, sunscreen, anti-aging agent, anti-wrinkle agent, anti-oxidant, anti-redness agent, moisturizing agent, exfoliating agent, or a combination thereof. Examples of suitable antioxidant additives include Olea europaea (olive) fruit extract, Terminalia ferdinandiana (kakadu plum) fruit extract, soy isoflavones, and juglans regia (walnut) seed extract. In some embodiments, the anti-oxidant may also act as an anti-wrinkle agent.

In these or other embodiments, the topical composition comprises the hydrator. In such embodiments, the hydrator may be selected from compounds known to penetrate the skin and absorb/retain water (e.g. sodium hyaluronate), as well as those useful for facilitating transport of water and/or hydrating compounds to or through the skin (e.g. liposomes). For example, in some embodiments, the topical composition comprises liposomes, such as those formed from or otherwise comprising omega fatty acids (e.g. omega 3, 6, and/or 9 fatty acids). Other hydrators may also be utilized, such as those known or otherwise sold under the name Acquacell, representing a blend of water, glycerin, Citrullus vulgaris (watermelon) fruit extract, Pyrus malus (apple) fruit extract, Lens esculenta (lentil) fruit extract, sodium pyrrolidone carboxylic acid (PCA), and sodium lactate, as well as Lubrajel (e.g. oil free), representing a blend of glycerin and glyceryl acrylate/acrylic acid copolymer and PVM/MA copolymer.

In some embodiments, the topical composition comprises the oil controller, such as zinc (PCA), a white willow bark extract, a witch hazel extract, a hexamethylene diisocyanate (HDI)/trimethylol hexyllactone crosspolymer, silica, or a combination thereof. In these or other embodiments, the topical composition comprises the exfoliant, such as an oat extract, a sugar or sugar derivative, or a combination thereof. In these or other embodiments, the topical composition comprises the anti-irritant, and wherein the anti-irritant comprises a glycyrrhizate salt, an astringent (e.g. a witch hazel extract); or a combination thereof. In these or other embodiments, the topical composition comprises one or more cosmetic additives, such as an acerola cherry extract, a biosaccharide gum, a fragrance, glycerin, butylene glycol, disodium EDTA, a polyoxyethylene ether of cetyl and/or stearyl alcohol or any combination thereof.

### Topical Moisturizer

The topical composition can optionally include a topical moisturizer (e.g., skin protectant). Any suitable topical skin protectant can be employed, provided the skin is effectively protected or moisturized and the skin protectant remains stable in the formulation. Suitable skin protectants include, e.g. aloe, lanolin, glycerin, calamine, Vitamin E, Vitamin E acetate, Vitamin C, allantoin, aluminum hydroxide gel, bismuth subnitrate, boric acid, calamine, cocoa butter, dimethicone, glycerin, kaolin, live yeast cell derivative, petrolatum, pyridoxine hydrochloride, shark liver oil, sodium bicarbonate, sulfur, tannic acid, topical starch, trolamine, white petrolatum, zinc acetate, zinc carbonate zinc oxide, zinc sulfate, shea butter, and any combination thereof.

As used herein, calamine is a pink powder of zinc oxide and a skin protectant containing about 98% zinc oxide and about 0.5% ferric oxide; aloe is the dried latex of leaves of Curaco Aloe (Aloe barbadenis Miller, Aloe vera Linne) or Cape Aloe (Aloe ferox Miller and hybrids), of the family Liliacaea; Vitamin E is 3,4-dihydro-2,5,7,8-tetramethyl-2-(4,8,12-trimethyltridecyl)-2H-1-benzopy-ran-6-ol; Vitamin E acetate is 3,4-dihydro-2,5,7,8-tetramethyl-2-(4,8,12-trimethyltridecyl)-2H-1-benzopy-ran-6-ol acetate; and lanolin is the fat-like secretion of the sebaceous glands of sheep (i.e., complex mixture of esters and polyesters of 33 high molecular weight alcohols and 36 fatty acids) which is deposited onto the wool fibers. In one embodiment, the topical moisturizer can be aloe and Vitamin E.

Aloe is commercially available as Aloe Vera Gel from Terry Laboratories (Melbourne, Fla.). Aloe Vera Gel is commercially available as Aloe Vera Gel 40.times. (20.0 wt. % solution in water), Aloe Vera Gel 1.times. (0.5 wt. % solution in water), Aloe Vera Gel (5.0 wt. % solution in water), or solid Aloe Vera. The solid Aloe Vera can be dissolved in a carrier, such as water, to the desired concentration. In addition, the commercially available forms of Aloe Vera are optionally available as decolorized Aloe Vera.

Any suitable amount of topical moisturizer can be employed, provided the suitable amount of topical moisturizer or skin protectant effectively protects or moisturizes the skin and the effective amount of skin protectant remains stable in the formulation over a prolonged period of time. The suitable and effective amount of topical moisturizer can depend in part upon the specific moisturizer or moisturizers present in the formulation. For example, Aloe Vera Gel can be present up to about 40.0 wt. % of the formulation. In one embodiment, Aloe Vera Gel can be present up to about 5.0 wt. % of the formulation. In one embodiment, Aloe Vera Gel can be present up to about 1.0 wt. % of the formulation. In addition, Vitamin E acetate can be present up to about 5 wt. % of the formulation. In one embodiment, Vitamin E acetate can be present up to about 1.0 wt. % of the formulation. In one embodiment, Vitamin E acetate can be present up to about 0.5 wt. % of the formulation.

In a specific embodiment, the nature and amount of the topical moisturizer is selected, such that it will be generally recognized as safe (GRAS) for topical use.

### Polyhydric Alcohol

The topical composition can optionally include one or more polyhydric alcohols. Suitable polyhydric alcohols include, e.g., ethylene glycol, propylene glycol, triethylene glycol, tetraethylene glycol, sorbitol, or any combination thereof. Specifically, the polyhydric alcohol can include propylene glycol. Any suitable amount of polyhydric alcohol can be employed. For example, when present in the topical composition, the polyhydric alcohol can be present up to about 35 wt. % of the topical composition, up to about 15 wt. % of the topical composition, or up to about 5 wt. % of the topical composition. In one embodiment, the polyhydric alcohol can be present in about 0.5 wt. % to about 5.0 wt. % of the topical composition.

### Water

The topical composition can optionally include water, e.g., deionized water (DI). Any suitable amount of water can be employed, provided the amount of water maintains the adhesiveness of the adhesive and maintains the appropriate stability of the topical composition. For example, deionized water can be present up to about 50 wt. % of the topical composition, up to about 40.0 wt. % of the formulation, or up to about 30.0 wt. % of the topical composition. In one embodiment, deionized water can be present up to about 20.0 wt. % of the topical composition. In one embodiment, deionized water can be present up to about 10.0 wt. % of the topical composition. In one embodiment, deionized water can be present in about 5.0 wt. % to about 15.0 wt. % of the topical composition.

### Personal Care Ingredient/Composition

In some embodiments the topical composition is formulated for use as a personal care composition, and further comprises a personal care ingredient. Examples of personal care compositions include antiperspirants and deodorants, skin care creams, skin care lotions, moisturizers, facial treatments (e.g. acne or wrinkle removers), personal and facial cleansers, bath oils, perfumes, colognes, sachets, sunscreens, pre-shave and after-shave lotions, shaving soaps and lathers, shampoos, conditioners, hair colorants, hair relaxants, hair sprays, mousses, hair gels, permanents, depilatories, cuticle coats, make-ups, color cosmetics, foundations, concealers, blushes, lipsticks, eyeliners, mascara, oil removers, color cosmetic removers, and medicament creams, pastes or sprays (e.g. for anti-acnes, dental hygienics, antibiotics, healing promotives, etc.).

The specific personal care ingredient, or a mixture of specific personal care ingredients, may be selected based on the type of personal care composition the composition is being formulated as. In these embodiments, the personal care ingredient may be a liquid, a solid, an encapsulated liquid, etc. Various examples of the personal care ingredient are described below. Any of these personal care ingredients, or a combination of two or more different personal care ingredients, may be utilized as the personal care ingredient. For clarity and consistency, “the personal care ingredient” encompasses embodiments where the composition includes but one or two or more personal care ingredients.

In specific embodiments, the personal care ingredient is an antiperspirant and/or deodorant (AP/DEO) agent. In these embodiments, the composition may be referred to as an antiperspirant and/or deodorant (AP/DEO) composition. Examples of antiperspirant agents and deodorant agents include aluminum chloride, aluminum zirconium tetrachlorohydrex GLY, aluminum zirconium tetrachlorohydrex PEG, aluminum chlorohydrex, aluminum zirconium tetrachlorohydrex PG, aluminum chlorohydrex PEG, aluminum zirconium trichlorohydrate, aluminum chlorohydrex PG, aluminum zirconium trichlorohydrex GLY, hexachlorophene, benzalkonium chloride, aluminum sesquichlorohydrate, sodium bicarbonate, aluminum sesquichlorohydrex PEG, chlorophyllincopper complex, triclosan, aluminum zirconium octachlorohydrate, zinc ricinoleate, and mixtures thereof.

In certain embodiments, the personal care ingredient comprises a skin care ingredient. If utilized to prepare the composition, the skin care ingredient is typically selected from water phase stabilizing agents, cosmetic biocides, conditioning agents (which may be silicone, cationic, hydrophobic, etc.), emollients, moisturizers, colorants, dyes, ultraviolet (UV) absorbers, sunscreen agents, antioxidants, fragrances, antimicrobial agents, antibacterial agents, antifungal agents, antiaging actives, anti-acne agents, skin-lightening agents, pigments, preservatives, pH controlling agents, electrolytes, chelating agents, plant extracts, botanical extracts, sebum absorbents, sebum control agents, vitamins, waxes, surfactants, detergents, emulsifiers, thickeners, propellant gases, skin protectants, film forming polymers, light scattering agents, and combinations thereof. In some of these embodiments, the composition may be referred to as a skin care composition, a cosmetic composition, a sunscreen, a shower gel, a soap, a hydrogel, a cream, a lotion, a balm, foundation, lipstick, eyeliner, a cuticle coat, a blush, etc., based on the particular personal care ingredients utilized. Various species of such skin care ingredients are set forth below, with similar and alternative species known by one of ordinary skill in the art.

Examples of emollients include volatile or non-volatile silicone oils; silicone resins such as polypropylsilsesquioxane and phenyl trimethicone; silicone elastomers such as dimethicone crosspolymer; alkylmethylsiloxanes such as C₃₀₋₄₅ alkyl methicone; volatile or non-volatile hydrocarbon compounds, such as squalene, paraffin oils, petrolatum oils and naphthalene oils; hydrogenated or partially hydrogenated polyisobutene; isoeicosane; squalane; isoparaff in; isododecane; isodecane or isohexa-decane; branched C₈-C₁₆ esters; isohexyl neopentanoate; ester oils such as isononyl isononanoate, cetostearyl octanoate, isopropyl myristate, palmitate derivatives (e.g. dextrin palmitate), stearates derivatives, diisostearyl malate, isostearyl isostearate and the heptanoates, octanoates, decanoates or ricinoleates of alcohols or of polyalcohols, or mixtures thereof; hydrocarbon oils of plant origin, such as wheatgerm, sunflower, grapeseed, castor, shea, avocado, olive, soybean, sweet almond, palm, rapeseed, cotton seed, hazelnut, macadamia, jojoba, blackcurrant, evening primrose; or triglycerides of caprylic/capric acids; higher fatty acids, such as oleic acid, linoleic acid or linolenic acid, and mixtures thereof.

Examples of waxes include hydrocarbon waxes such as beeswax, lanolin wax, rice wax, carnauba wax, candelilla wax, microcrystalline waxes, paraffins, ozokerite, polyethylene waxes, synthetic wax, ceresin, lanolin, lanolin derivatives, cocoa butter, shellac wax, bran wax, capok wax, sugar cane wax, montan wax, whale wax, bayberry wax, silicone waxes (e.g. polymethylsiloxane alkyls, alkoxys and/or esters, C₃₀₋₄₅ alkyldimethylsilyl polypropylsilsesquioxane), stearyl dimethicone, alkylmethylsiloxanes including long-chain alkyl groups in alkylmethylsiloxy units, and mixtures thereof.

Examples of moisturizers include lower molecular weight aliphatic diols such as propylene glycol and butylene glycol; polyols such as glycerine and sorbitol; and polyoxyethylene polymers such as polyethylene glycol 200; hyaluronic acid and its derivatives, and mixtures thereof.

Examples of thickeners include acrylamide copolymers, acrylate copolymers and salts thereof (such as sodium polyacrylate), xanthan gum and derivatives, cellulose gum and cellulose derivatives (such as methylcellulose, methylhydroxypropylcellulose, hydroxyethylcellulose, hydroxypropylcellulose, polypropylhydroxyethylcellulose), starch and starch derivatives (such as hydroxyethylamylose and starch amylase), polyoxyethylene, carbomer, alginates (such as sodium alginate), arabic gum, cassia gum, carob gum, scleroglucan gum, gellan gum, rhamsan gum, karaya gum, carrageenan gum, guar gum and guar gum derivatives, cocamide derivatives (including cocamidopropyl betaine and cocamide MIPA), alkyl alcohols (such as cetearyl alcohol, stearyl alcohol, and other fatty alcohols), gelatin, PEG-derivatives, saccharides (such as fructose, glucose) and saccharides derivatives (such as PEG-120 methyl glucose diolate), and mixtures thereof.

Examples of water phase stabilizing agents include electrolytes (e.g. alkali metal salts and alkaline earth salts, especially the chloride, borate, citrate, and sulfate salts of sodium, potassium, calcium and magnesium, as well as aluminum chlorohydrate, and polyelectrolytes, especially hyaluronic acid and sodium hyaluronate), polyols (glycerine, propylene glycol, butylene glycol, and sorbitol), alcohols such as ethyl alcohol, and hydrocolloids, and mixtures thereof.

Examples of pH controlling agents include any water soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, citric acid, and mixtures thereof.

Example of preservatives and cosmetic biocides include paraben derivatives (e.g. methylparaben, propylparaben), hydantoin derivatives, chlorhexidine and its derivatives, imidazolidinyl urea, diazolidinyl urea, phenoxyethanol, silver derivatives, salicylate derivatives, triclosan, ciclopirox olamine, hexamidine, oxyquinoline and its derivatives, PVPiodine, zinc salts and derivatives such as zinc pyrithione, methylchloroisothiazolinone, methylisothiazolinone, and mixtures thereof.

Examples of sebum absorbents or sebum control agents include silica silylate, silica dimethyl silylate, dimethicone/vinyl dimethicone crosspolymer, polymethyl methacrylate, cross-linked methylmethacrylate, aluminum starch octenylsuccinate, and mixtures thereof.

Examples of pigments and colorants include surface treated or untreated iron oxides, surface treated or untreated titanium dioxide, surface treated or untreated mica, silver oxide, silicates, chromium oxides, carotenoids, carbon black, ultramarines, chlorophyllin derivatives and yellow ocher. Examples of organic pigments include aromatic types including azo, indigoid, triphenylmethane, anthraquinone, and xanthine dyes which are designated as D&C and FD&C blues, browns, greens, oranges, reds, yellows, etc., and mixtures thereof. Surface treatments include those treatments based on lecithin, silicone, silanes, fluoro compounds, and mixtures thereof.

Examples of silicone conditioning agents include silicone oils such as dimethicone; silicone gums such as dimethiconol; silicone resins such as trimethylsiloxy silicate, polypropyl silsesquioxane; silicone elastomers; alkylmethylsiloxanes; organomodified silicone oils, such as amodimethicone, aminopropyl phenyl trimethicone, phenyl trimethicone, trimethyl pentaphenyl trisiloxane, silicone quaternium-16/glycidoxy dimethicone crosspolymer, silicone quaternium-16; saccharide functional siloxanes; carbinol functional siloxanes; silicone polyethers; siloxane copolymers (divinyldimethicone/dimethicone copolymer); acrylate or acrylic functional siloxanes; and mixtures or emulsions thereof.

Examples of cationic conditioning agents include guar derivatives such as hydroxypropyltrimethylammonium derivative of guar gum; cationic cellulose derivatives, cationic starch derivatives; quaternary nitrogen derivatives of cellulose ethers; homopolymers of dimethyldiallyl ammonium chloride; copolymers of acrylamide and dimethyldiallyl ammonium chloride; homopolymers or copolymers derived from acrylic acid or methacrylic acid which contain cationic nitrogen functional groups attached to the polymer by ester or amide linkages; polymeric quaternary ammonium salts of hydroxyethyl cellulose reacted with a fatty alkyl dimethyl ammonium substituted epoxide; polycondensation products of N,N′-bis-(2,3-epoxypropyl)-piperazine or piperazine-bis-acrylamide and piperazine; and copolymers of vinylpyrrolidone and acrylic acid esters with quaternary nitrogen functionality. Specific materials include the various polyquats, e.g. Polyquaternium-7, Polyquaternium-8, Polyquaternium-10, Polyquaternium-11, and Polyquaternium-23. Other categories of conditioners include cationic surfactants such as cetyl trimethylammonium chloride, cetyl trimethylammonium bromide, stearyltrimethylammonium chloride, and mixtures thereof. In some instances, the cationic conditioning agent is also hydrophobically modified, such as hydrophobically modified quaternized hydroxyethylcellulose polymers; cationic hydrophobically modified galactomannan ether; and mixtures thereof.

Examples of hydrophobic conditioning agents include guar derivatives; galactomannan gum derivatives; cellulose derivatives; and mixtures thereof.

UV absorbers and sunscreen agents include those which absorb ultraviolet light between 290-320 nanometers (the UV-B region) and those which absorb ultraviolet light in the range of 320-400 nanometers (the UV-A region), as well as blue light absorbers, as known in the art.

Some examples of sunscreen agents are aminobenzoic acid, cinoxate, diethanolamine methoxycinnamate, digalloyl trioleate, dioxybenzone, ethyl 4-[bis(Hydroxypropyl)] aminobenzoate, glyceryl aminobenzoate, homosalate, lawsone with dihydroxyacetone, menthyl anthranilate, octocrylene, ethylhexyl methoxycinnamate (or octyl methoxycinnamate), octyl salicylate (or ethylhexyl salicylate), oxybenzone, padimate O, phenylbenzimidazole sulfonic acid, red petrolatum, sulisobenzone, titanium dioxide, trolamine salicylate, and mixtures thereof.

Some examples of UV absorbers are acetaminosalol, allantoin PABA, benzalphthalide, benzophenone, benzophenone 1-12, 3-benzylidene camphor, benzylidenecamphor hydrolyzed collagen sulfonamide, benzylidene camphor sulfonic Acid, benzyl salicylate, bornelone, bumetriozole, butyl methoxydibenzoylmethane, butyl PABA, ceria/silica, ceria/silica talc, cinoxate, DEA-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl p-methoxycinnamate, isopropylbenzyl salicylate, isopropyl dibenzolylmethane, isopropyl methoxycinnamate, octyl methoxycinnamate, menthyl anthranilate, menthyl salicylate, 4-methylbenzylidene, camphor, octocrylene, octrizole, octyl dimethyl PABA, ethyl hexyl methoxycinnamate, octyl salicylate, octyl triazone, PABA, PEG-25 PABA, pentyl dimethyl PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, TEA-phenylbenzimidazole sulfonate, TEA-salicylate, terephthalylidene dicamphor sulfonic acid, titanium dioxide, triPABA panthenol, urocanic acid, VA/crotonates/methacryloxybenzophenone-1 copolymer, and mixtures thereof.

Examples of skin protectants include allantoin, aluminium acetate, aluminium hydroxide, aluminium sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, zinc oxide, and mixtures thereof.

Examples of dyes include 1-acetoxy-2-methylnaphthalene; acid dyes; 5-amino-4-chloro-o-cresol; 5-amino-2,6-dimethoxy-3-hydroxypyridine; 3-amino-2,6-dimethylphenol; 2-amino-5-ethylphenol HCl; 5-amino-4-fluoro-2-methylphenol sulfate; 2-amino-4-hydroxyethylaminoanisole; 2-amino-4-hydroxyethylaminoanisole sulfate; 2-amino-5-nitrophenol; 4-amino-2-nitrophenol; 4-amino-3-nitrophenol; 2-amino-4-nitrophenol sulfate; m-aminophenol HCl; p-aminophenol HCl; m-aminophenol; o-aminophenol; 4,6-bis(2-hydroxyethoxy)-m-phenylenediamine HCl; 2,6-bis(2-hydroxyethoxy)-3,5-pyridinediamine HCl; 2-chloro-6-ethylamino-4-nitrophenol; 2-chloro-5-nitro-N-hydroxyethyl p-phenylenediamine; 2-chloro-p-phenylenediamine; 3,4-diaminobenzoic acid; 4,5-diamino-1-((4-chlorophenyl)methyl)-1H-pyrazole-sulfate; 2,3-diaminodihydropyrazolo pyrazolone dimethosulfonate; 2,6-diaminopyridine; 2,6-diamino-3-((pyridin-3-yl)azo)pyridine; dihydroxyindole; dihydroxyindoline; N,N-dimethyl-p-phenylenediamine; 2,6-dimethyl-p-phenylenediamine; N,N-dimethyl-p-phenylenediamine sulfate; direct dyes; 4-ethoxy-m-phenylenediamine sulfate; 3-ethylamino-p-cresol sulfate; N-ethyl-3-nitro PABA; gluconamidopropyl aminopropyl dimethicone; Haematoxylon brasiletto wood extract; HC dyes; Lawsonia inermis (Henna) extract; hydroxyethyl-3,4-methylenedioxyaniline HCl; hydroxyethyl-2-nitro-p-toluidine; hydroxyethyl-p-phenylenediamine sulfate; 2-hydroxyethyl picramic acid; hydroxypyridinone; hydroxysuccinimidyl C₂₁-C₂₂ isoalkyl acidate; isatin; Isatis tinctoria leaf powder; 2-methoxymethyl-p-phenylenediamine sulfate; 2-methoxy-p-phenylenediamine sulfate; 6-methoxy-2,3-pyridinediamine HCl; 4-methylbenzyl 4,5-diamino pyrazole sulfate; 2,2′-methylenebis 4-aminophenol; 2,2′-methylenebis-4-aminophenol HCl; 3,4-methylenedioxyaniline; 2-methylresorcinol; methylrosanilinium chloride; 1,5-naphthalenediol; 1,7-naphthalenediol; 3-nitro-p-Cresol; 2-nitro-5-glyceryl methylaniline; 4-nitroguaiacol; 3-nitro-p-hydroxyethylaminophenol; 2-nitro-N-hydroxyethyl-p-anisidine; nitrophenol; 4-nitrophenyl aminoethylurea; 4-nitro-o-phenylenediamine dihydrochloride; 2-nitro-p-phenylenediamine dihydrochloride; 4-nitro-o-phenylenediamine HCl; 4-nitro-m-phenylenediamine; 4-nitro-o-phenylenediamine; 2-nitro-p-phenylenediamine; 4-nitro-m-phenylenediamine sulfate; 4-nitro-o-phenylenediamine sulfate; 2-nitro-p-phenylenediamine sulfate; 6-nitro-2,5-pyridinediamine; 6-nitro-o-toluidine; PEG-3 2,2′-di-p-phenylenediamine; p-phenylenediamine HCl; p-phenylenediamine sulfate; phenyl methyl pyrazolone; N-phenyl-p-phenylenediamine HCl; pigment blue 15:1; pigment violet 23; pigment yellow 13; pyrocatechol; pyrogallol; resorcinol; sodium picramate; sodium sulfanilate; solvent yellow 85; solvent yellow 172; tetraaminopyrimidine sulfate; tetrabromophenol blue; 2,5,6-triamino-4-pyrimidinol sulfate; 1,2,4-trihydroxybenzene.

Examples of fragrances include perfume ketones and perfume aldehydes. Illustrative of the perfume ketones are buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, Camphor, Fenchone, Alpha-Ionone, Beta-Ionone, Gamma-Methyl so-called Ionone, Fleuramone, Dihydrojasmone, Cis-Jasmone, Iso-E-Super, Methyl-Cedrenyl-ketone or Methyl-Cedrylone, Acetophenone, Methyl-Acetophenone, Para-Methoxy-Acetophenone, Methyl-Beta-Naphtyl-Ketone, Benzyl-Acetone, Benzophenone, Para-Hydroxy-Phenyl-Butanone, Celery Ketone or Livescone, 6-Isopropyldecahydro-2-naphtone, Dimethyl-Octenone, Freskomenthe, 4-(1-Ethoxyvinyl)-3,3,5,5,-tetramethyl-Cyclohexanone, Methyl-Heptenone, 2-(2-(4-Methyl-3-cyclohexen-1-yl)propyl)-cyclopentanone, 1-(p-Menthen-6(2)-yl)-1-propanone, 4-(4-Hydroxy-3-methoxyphenyl)-2-butanone, 2-Acetyl-3,3-Dimethyl-Norbornane, 6,7-Dihydro-1,1,2,3,3-Pentamethyl-4(5H)-Indanone, 4-Damascol, Dulcinyl or Cassione, Gelsone, Hexylon, Isocyclemone E, Methyl Cyclocitrone, Methyl-Lavender-Ketone, Orivon, Para-tertiary-Butyl-Cyclohexanone, Verdone, Delphone, Muscone, Neobutenone, Plicatone, Veloutone, 2,4,4,7-Tetramethyl-oct-6-en-3-one, and Tetrameran. The fragrance may be derived or extracted from flowers, seeds, leaves, and/or roots of plants, seaweed, etc. The fragrance may be extracted from an animal, e.g. from a secretion gland, and may be a musk or sperm oil. The fragrance may also be artificially synthesized, e.g. menthol, acetate, vanilla, etc.

In specific embodiments, the perfume ketones are selected for odor character from Alpha Damascone, Delta Damascone, Iso Damascone, Carvone, Gamma-Methyl-Ionone, Iso-E-Super, 2,4,4,7-Tetramethyl-oct-6-en-3-one, Benzyl Acetone, Beta Damascone, Damascenone, methyl dihydrojasmonate, methyl cedrylone, and mixtures thereof.

In specific embodiments, the perfume aldehyde is selected for odor character from adoxal; anisic aldehyde; cymal; ethyl vanillin; florhydral; helional; heliotropin; hydroxycitronellal; koavone; lauric aldehyde; lyral; methyl nonyl acetaldehyde; P. T. bucinal; phenyl acetaldehyde; undecylenic aldehyde; vanillin; 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl propanal, 2-methyl-4-(2,6,6-trimethyl-2(1)-cyclohexen-1-yl) butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclohexene-1-carboxaldehyde, 5 or 6 methoxy 10 hexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-decenal, 2,6-nonadienal, paratolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl)benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, hexanal, trans-2-hexenal, 1-p-menthene-q-carboxaldehyde and mixtures thereof.

Examples of antioxidants are acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, phydroxyanisole, BHT, tbutyl hydroquinone, caffeic acid, Camellia sinensis oil, chitosan ascorbate, chitosan glycolate, chitosan salicylate, chlorogenic acids, cysteine, cysteine HCl, decyl mercaptomethylimidazole, erythorbic acid, diamylhydroquinone, ditbutylhydroquinone, dicetyl thiodipropionate, dicyclopentadiene/tbutylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, distearyl thiodipropionate, ditridecyl thiodipropionate, dodecyl gallate, ethyl ferulate, ferulic acid, hydroquinone, hydroxylamine HCl, hydroxylamine sulfate, isooctyl thioglycolate, kojic acid, madecassicoside, magnesium ascorbate, magnesium ascorbyl phosphate, melatonin, methoxyPEG7 rutinyl succinate, methylene ditbutylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl phosphate, sodium bisulfite, sodium erythorbate, sodium metabisulfide, sodium sulfite, sodium thioglycolate, sorbityl furfural, tea tree (Melaleuca aftemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth5, tocophereth10, tocophereth12, tocophereth18, tocophereth50, tocopherol, tocophersolan, tocopheryl linoleate, tocopheryl nicotinate, tocoquinone, otolyl biguanide, tris(nonylphenyl) phosphite, ubiquinone, zinc dibutyldithiocarbamate, and mixtures thereof.

Examples of propellant gases include carbon dioxide, nitrogen, nitrous oxide, volatile hydrocarbons such as butane, isobutane, or propane, and chlorinated or fluorinated hydrocarbons such as dichlorodifluoromethane and dichlorotetrafluoroethane or dimethylether; and mixtures thereof.

In a specific embodiment, the functional composition is a sunscreen. In these embodiments, personal care ingredient comprises the sunscreen agent. The sunscreen agent may be, for example, a sunscreen additive, an SPF booster, a photostabilizer, a film-forming polymer, etc. The sunscreen may be also or alternatively be utilized in sunless tanning applications. Specific examples of sunscreen agents are set forth above.

In other embodiments, the personal care ingredient comprises a hair care ingredient. In these embodiments, the composition may be referred to as a hair care composition. If utilized to prepare the functional composition, the hair care ingredient is typically selected from conditioning agents (which may be silicone, cationic, hydrophobic, etc.), colorants, dyes, ultraviolet (UV) absorbers, preservatives, plant extracts, fatty alcohols, vitamins, fragrance, anti-dandruff agents, color care additives, pearlising agents, pH controlling agents, electrolytes, chelating agents, styling agents, ceramides, amino-acid derivatives, suspending agents, surfactants, detergents, emulsifiers, thickeners, oxidizing agents, reducing agents, film-forming polymers, and combinations thereof. With some of these hair care embodiments, the composition may be referred to as a shampoo, a rinse-off conditioner, a leave-in conditioner, a gel, a pomade, a serum, a spray, a coloring product, or mascara. Examples of many of these hair care ingredients are set forth above as suitable personal care ingredients.

Examples of oxidizing agents are ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, zinc peroxide, and mixtures thereof. Examples of reducing agents are ammonium bisulfite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, zinc formaldehyde sulfoxylate, and mixtures thereof.

Examples of antidandruff agents include pyridinethione salts, selenium compounds such as selenium disulfide, and soluble antidandruff agents, and mixtures thereof.

In certain embodiments, the personal care ingredient comprises a film-forming polymer, which may be utilized as the personal care ingredient whether the functional composition is utilized for skin care, hair care, etc. “Film-forming polymer,” as used herein, means a polymer or oligomer which is capable of, by itself or optionally in the presence of a film-forming agent, forming a film on a substrate. The film-forming polymer may form the film upon an application of a curing condition, e.g. the application of heat, exposure to atmospheric conditions, etc. Alternatively, the film-forming polymer may form the film upon evaporation of any carrier vehicle in which the film-forming polymer may optionally be disposed. The film-forming polymer may undergo a reaction, e.g. the film-forming polymer may become cross-linked or otherwise include additional bonds, when forming the film. However, the film-forming polymer may form the film in the absence of such a reaction. The film-forming polymer may be a gelling agent. The film-forming polymer is particularly advantageous when the functional composition is formulated as a sunscreen, although the personal care ingredient may comprise the film-forming polymer in other forms of functional composition as well.

The substrate on which the film is formed may be any substrate, although the substrate is generally a portion of a mammal, particularly a human, as described in greater detail below with reference to the treatment method. Specific examples of suitable substrates include skin, hair, and nails.

### Personal Care Active

In various embodiments, the personal care ingredient may comprise or be referred to as a personal care active, a health care active, or combination thereof (collectively “active” or “actives”). As used herein, a “personal care active” means any compound or mixtures of compounds that are known in the art as additives in personal care formulations, typically for providing a cosmetic and/or aesthetic benefit. A “healthcare active” means any compound or mixtures of compounds that are known in the art to provide a pharmaceutical or medical benefit. Thus, “healthcare active” includes materials considered as an active ingredient or active drug ingredient as generally used and defined by the United States Department of Health & Human Services Food and Drug Administration, contained in Title 21, Chapter I, of the Code of Federal Regulations, Parts 200-299 and Parts 300-499. Specific personal care actives and health care actives are described below. These personal care actives and health care actives may constitute the personal care ingredient whether the personal care ingredient is utilized to form, for example, the AP/DEO composition, the skin care composition, the hair care composition, the nail care composition, and/or the tooth care composition. For example, in various embodiments, the same personal care ingredient may be utilized to form either the hair care composition or the skin care composition. As understood in the art, at least some of the personal care actives described below are species of certain personal care ingredients introduced above with respect to the skin care composition, the hair care composition, the nail care composition, and the tooth care composition, respectively. For example, numerous species of plant or vegetable extracts are described below, which are exemplary examples of plant extracts set forth above as suitable personal care ingredients. The active ingredients or actives described below may constitute the personal care ingredient of the functional composition or may be utilized in combination therewith.

Useful personal care active ingredients for use in the functional composition include vitamins and vitamin derivatives, including “pro-vitamins”. Vitamins useful herein include, but are not limited to, Vitamin A1, retinol and its derivatives (e.g. C₂-C₁₈ esters of retinol, trans-retinol, 1,3-cis-retinol, 11-cis-retinol, 9-cis-retinol, and 3,4-didehydro-retinol, as well as trans retinoic acids (i.e., retinoids)), Vitamin B1, Vitamin B2, Pro Vitamin B5, panthenol, Vitamin B6, Vitamin B12, niacin, Vitamin C and its derivatives, vitamin E, tocopherol, esters of vitamin E, folic acid, biotin, pantothenic acid, and mixtures thereof. Other suitable vitamins and the International Nomenclature Cosmetic Ingredient (INCI) names for the vitamins suitable for use in the topical composition include ascorbyl dipalmitate, ascorbyl methylsilanol pectinate, ascorbyl palmitate, ascorbyl stearate, ascorbyl glucocide, sodium ascorbyl phosphate, sodium ascorbate, disodium ascorbyl sulfate, potassium (ascorbyl/tocopheryl) phosphate, retinyl acetate, retinyl palmitate, retinyl propionate, □-tocopherol, tocophersolan, tocopheryl acetate, tocopheryl linoleate, tocopheryl nicotinate, and tocopheryl succinate, as well as combinations thereof.

The personal care active can be a protein, such as an enzyme. Enzymes include commercially available types, improved types, recombinant types, wild types, variants not found in nature, and mixtures thereof. For example, suitable enzymes include hydrolases, cutinases, oxidases, transferases, reductases, hemicellulases, esterases, isomerases, pectinases, lactases, peroxidases, laccases, catalases, and mixtures thereof. Hydrolases include proteases (bacterial, fungal, acid, neutral or alkaline), amylases (alpha or beta), lipases, mannanases, cellulases, collagenases, lisozymes, superoxide dismutase, catalase, and mixtures thereof. Proteases include trypsin, chymotrypsin, pepsin, pancreatin and other mammalian enzymes; papain, bromelain and other botanical enzymes; subtilisin, epidermin, nisin, naringinase (L-rhammnosidase) urokinase and other bacterial enzymes. Lipase include triacyl-glycerol lipases, monoacyl-glycerol lipases, lipoprotein lipases, e.g. steapsin, erepsin, pepsin, other mammalian, botanical, bacterial lipases and purified ones.

The personal care composition may be of any form, such as a cream, gel, powder, paste, or freely pourable liquid. The personal care composition may exhibit improved application and cosmetic properties, e.g. reduced tackiness and stickiness, as well as improved clarity/low residue properties, etc. In such embodiments, the personal care composition is formulated to be cosmetic, therapeutic, and functional with respect to a portion of a body to which the personal care composition is applied, or some combinations thereof, as described above with respect to the topical composition itself.

In general, the personal care composition is formulated with a carrier that permits application in a conventional form, such as a liquid, rinse, lotion, cream, paste, gel, foam, mousse, ointment, spray, aerosol, soap, stick, soft solid, or solid gel, e.g. depending on the intended use. What constitutes a suitable carrier for formulating the personal care composition is readily apparent to one of ordinary skill in the art, and may be selected from those carriers exemplified herein.

The personal care composition may be in any form, either liquid or non-liquid (semi-solid, soft solid, solid, etc.). For example, the personal care composition may be a paste, a solid, a gel, or a cream. Additionally, regardless of how the emulsion was prepared, the personal care composition formed from the emulsion may itself be an emulsion, such as an oil-in-water or water-in-oil emulsion, a multiple emulsion, such as an oil-in-water-in-oil emulsion or a water-in-oil-in-water emulsion, or a solid, rigid or supple gel, including anhydrous gels. The personal care composition can also be in a form chosen from a translucent anhydrous gel and a transparent anhydrous gel. The personal care composition may, for example, comprise an external or continuous fatty phase. The personal care composition may be anhydrous. In some instances, the personal care composition can be a molded composition or cast as a stick or a dish. In specific embodiments, the personal care composition comprising the emulsion is a molded poured stick. In such embodiments, the personal care composition (e.g. in stick form) may behave as deformable, flexible elastic solid, having increased elastic softness on application.

The personal care composition can be used by any method, such as via application to a human body (e.g. skin or hair) by hand or with an applicator (e.g. a brush or sprayer). In some embodiments, the personal care composition may be intended to be removed after such application, e.g. by washing, wiping, peeling, and the like, or combinations thereof.

### Active Amount/Concentration

The topical composition, in any of the forms described herein, may comprise any amount of the cannabis extract, which will typically be limited by nature of the particular cannabis phytocompound content therein. Specifically, daily exposure limits are specified by numerous regulatory/governing organizations, and are being regularly investigated and updated based on the increasing understanding of biological effects of such compounds. As such, particular amounts and limits of any given cannabis phytocompound present in the topical composition may vary, even irrespective of potency (i.e., where a more-potent content is prohibited by law or rule). As but one example, the topical composition may be formulated to comprise up to 400 mg of CBD per packaged amount of the topical composition, e.g. in a single product package form (i.e., a batch form comprising multiple application amounts). In some embodiments, the topical composition may be formulated to comprise from 200 to 400 mg, alternatively from 250 to 400 mg, alternatively of 250, 300, 350, or 400 mg of CBD per packaged amount of the topical composition.

In some embodiments, the topical composition is formulated to comprise a particular concentration of one or more cannabis phytocompounds. For example, in some such embodiments the topical composition is formulated to comprise a CBD content of from 50 to 80 wt. %, based on the total weight of the topical composition. However, as described above, such amounts may vary based on the given form of the topical composition. For example, in specific embodiments, the topical composition is formulated as a skincare composition comprising a CBD content of from 0.4 to 0.7% (wt./wt.). In some embodiments, the topical composition comprises from 100 to 200 mg CBD per ounce. For example, in certain such embodiments the topical composition is formulated as a 2 oz product comprising from 250-400 mg CBD.

In some embodiments, the topical composition is free from, alternatively substantially free from, a CBD isolate. For example, in certain such embodiments the cannabis extract of the topical composition comprises a Cannabis sativa flower or stalk extract comprising a CBD content of less than 90%, alternatively less than 85%, alternatively less than 80%, by weight.

The limitations with respect to CBD in the preceding description concerning cannabis phytocompound content in the topical composition may apply to any given cannabis phytocompound, the amount and contents of which will be independently selected by those of skill in the art. Particular amounts and examples of such contents are described and exemplified herein, and may be validated and/or determined using standard methodology for safety and efficacy known in the art.

It is to be appreciated that certain components or additives may be classified under different terms of art and just because a component or additive is classified under such a term does not mean that they are limited to that functionAdditional ingredients for optional use in the composition, e.g. when adapted for topical or oral administration, are described in U.S. Pat. Nos. 5,747,006; 5,980,904; 6,994,874; 7,060,304; 7,247,321; 7,348,034; 7,364,759; 7,700,110; 7,722,904; 8,202,556; 8,916,212; 9,445,975; 9,801,809; 10,307,366; 10,532,024; and 10,537,516; and in U.S. Publication Nos. 2006/0257509; 2007/0224154; 2008/0081082; 2008/0124409; 2013/0302265; 2017/0252293; 2017/0281666; 2018/0200285; 2019/0083566; 2019/0160117; 2020/0171117; 2020/0383898; 2021/0017240; and 2021/0212926; the disclosures of which are hereby incorporated by reference in their entirety.

In addition to the forms of the functional composition described above, it will be appreciated that the cannabis composition may be formulated into for dermal (or trans-dermal) delivery/administration to a subject. In such embodiments, the topical composition may be prepared in a form for use in, on, with, and/or as an ointment, patch (i.e., a transdermal patch), intradermal implant, subcutaneous implant, or transdermal implant. As used herein, “dermal delivery” or “dermal administration” refers to a route of administration wherein the pharmaceutical dosage form is taken to, or through, the dermis (i.e., layer of skin between the epidermis (with which it makes up the cutis) and subcutaneous tissues). The transdermal patch is typically provided as a medicated adhesive patch that is placed on the skin to deliver a specific dose of the cannabis extract through the skin and into the bloodstream of a subject. Such patches are known in the art, and may be utilized with the functional composition generally to provide a therapeutic route of administration to subjects who experience dysphagia (i.e., difficulty swallowing), or even purely for sake of convenient application.

The various forms of the functional composition, both in terms of the consumable and topical compositions described above, will be best understood in view of the exemplary compositions below. Such compositions may be tailors for specific application and/or dosage regimens, which will be readily understood by those of skill in the art.

### Preparation Method

A method of preparing the functional composition is further provided. The method comprises combining the cannabis composition with a carrier (e.g. a pharmaceutically- and/or food-acceptable carrier), thereby preparing the functional composition. In certain embodiments, the cannabis composition is in the form of a CBD isolate. In other embodiments, the cannabis composition is free from, alternatively substantially free from, CBD isolate, but instead comprises a cannabis extract comprising less than 90, alternatively less than 85, alternatively less than 80 wt. % CBD, based on total weight of the cannabis extract.

The method is not particularly limited beyond combining the cannabis composition with the carrier, and may be carried out using any of the techniques known in the art and/or described herein. The carrier will be selected based on the desired form and intended use of the functional composition, which will be understood in view of the description and exemplary carriers set forth above.

### Treatment Method

A method of ameliorating (e.g. treating, preventing) a condition with the functional composition (the “treatment method”) is also provided. The treatment method comprises administering the functional composition to a subject in an amount effective to elicit a biological response from the subject. In certain embodiments, the treatment method comprises administering the functional composition topically to the subject, e.g. in the form of the personal care product. In other embodiments, the treatment method comprises administering the functional composition enterally to the subject, e.g. in a consumable form such as the oral and/or dietary supplement. The particular dosage regimen will vary, i.e., depending on the form and type of the functional composition being utilized.

For example, in some embodiments, the functional composition of the treatment method is a foodstuff, and administered in an amount defined by serving size. For example, the functional composition may be in the form of a gummy and administered in an amount of from 1 to 5 gummies per day, such as 2 or 3 gummies per day. In some embodiments, the functional composition of the treatment method is in the form of a tablet or other similar single- or split-dosage from, and administered in an amount of 1 to 3 tablets (or softgels) per day. For example, the functional composition may be in the form of a gummy and administered in an amount of from 2 gummies per day.

The composition may be administered as needed, daily, several times per day or in any suitable regimen such that the desired outcome is achieved. In the treatment method, the frequency of administration can depend on several factors, including the desired level of prevention or amelioration. Generally, a regimen includes administration of the functional composition to the subject once or twice daily, e.g. to include an administration in the morning and/or an administration in the evening.

The amount of composition administered to the subject during each administration may depend on several factors including level of desired results and the specific composition being utilized. In general, the composition is administered in a therapeutically or physiologically effective amount.

The treatment method may include administering the functional composition in an effective amount to elicit a biological or medicinal response in a tissue, in a system, in an animal, in a subject (i.e., a human subject), etc. Such response need not by determinative of the benefit achieved, e.g. where a desired outcome is reached in part or wholly due to anecdotal and/or entourage effects. Regardless, the effective amount may include a therapeutically effective amount of one or more cannabis phytocompounds for the alleviation of one or more symptoms of a disease or condition, such as any of those described above. Likewise, the effective amount may include a prophylactically effective amount of the one or more cannabis phytocompounds for a reduction of a severity and/or likelihood of one or more symptoms of a disease or condition. For example, the functional composition may be further defined as a topical CBD composition, and may be administered in an effective amount to reduce pain and/or inflammation. Specific examples of functional compositions suitable for use in the treatment method are included below.

### EXAMPLES

The following examples, illustrating embodiments of this disclosure, are intended to illustrate and not to limit the invention. Unless otherwise noted, all reactions are carried out under air, and all solvents, substrates, and reagents are purchased or otherwise obtained from various commercial suppliers and utilized as received. All components are mixed or blended according to conventional combination routes.

### Oral Supplements

Example 1: Tincture 1. An oral supplement in the form of a flavored tincture is prepared using the components and parameters shown Table 2 below to give Tincture 1.

TABLE 2
Components of Example 1: Tincture 1

| | per serving, | | |
|---|---|---|---|
| Ingredient | 1 dropper (mg) | % | Function |
| MCT oil (coconut or palm) | 600 | 66.67 | Carrier |
| Natural Peppermint Flavor | 100 | 11.11 | Flavor |
| Hemp Extract (cont. CBD) | 100 | 11.11 | Active |
| Mixed tocopherols | 50 | 5.56 | Antioxidant |
| Luo Han Guo | 50 | 5.56 | sweetener |
| Total: | 900 | 100 | |

Alternative carriers include olive oil and hemp seed oil. Alternative flavors include berry, lemon, and chocolate mint.

Example 2: Tincture 2. An oral supplement in the form of an unflavored tincture is prepared using the components and parameters shown Table 3 below to give Tincture 2.

TABLE 3
Components of Example 2: Tincture 2

| | per serving, | | |
|---|---|---|---|
| Ingredient | 1 dropper (mg) | % | Function |
| Extra virgin Olive Oil | 600 | 66.67 | Carrier |
| Hemp Seed Oil | 200 | 22.22 | Flavor |
| Hemp Extract (cont. CBD) | 100 | 11.11 | Active |
| Total: | 900 | 100 | |

The tinctures of Examples 1-2 are administered to a subject via application from a dropper according to the following directions/protocol:

1. Take 30 drops (1 mL) per day (a full dropper);
2. Place 20 drops (approximately 3/4 full dropper) under the tongue as needed.

In each tincture, a typical CBD content is 8-25 mg/mL.

Example 3: Softgel 1. An oral supplement in the form of a softgel is prepared using the components and parameters shown Table 4 below to give Softgel 1.

TABLE 4
Components of Example 3: Softgel 1

| Ingredient | per softgel (mg) | % | Function |
|---|---|---|---|
| MCT oil (coconut or palm) | 500 | 55.56 | Carrier |
| Gelatin (bovine; Halal certified) | 170 | 18.89 | Shell material |
| Glycerin | 80 | 8.89 | Shell solvent |
| Water | 40 | 4.44 | Shell solvent |
| Yellow beeswax | 20 | 2.22 | Stabilizer |
| Soy Lecithin | 20 | 2.22 | Emulsifier |
| Hemp Extract (cont. CBD, 15 mg) | 60 | 6.67 | Active |
| Caramel Color | 10 | 1.11 | Shell colorant |
| Total: | 900 | 100 | |

Example 4: Softgel 2. An oral supplement in the form of a softgel (vegetarian) is prepared using the components and parameters shown Table 5 below to give Softgel 2.

TABLE 5
Components of Example 4: Softgel 2

| Ingredient | per softgel (mg) | % | Function |
|---|---|---|---|
| Modified Food Starch | 200 | 29.63 | Shell material |
| Glycerin | 50 | 7.41 | Shell solvent |
| Carrageenan | 40 | 5.93 | Shell material |
| Water | 20 | 2.96 | Shell solvent |
| Annatto | 5 | 0.74 | Shell colorant |
| Sunflower oil | 300 | 44.44 | Carrier |
| Hemp Extract | 60 | 8.89 | Active |
| (cont. CBD, 15 mg) | | | |
| Total: | 675 | 100 | |

The softgels of Examples 3-4 are administered to a subject orally, via ingestion of one softgel per day (e.g. with water).

In each softgel, a typical CBD content is 5-20 mg.

For the above softgels, alternative carriers include safflower oil, sunflower oil, hemp seed oil, olive oil, and flaxseed oil. Flavors including mint and berry may be added. Natural colorants including sodium copper chlorophyllin may be used for colored-softgels. Other active ingredients for similar function may be added, including turmeric, boswellia for joint health, melatonin for sleep, and ashwagandha for stress and cognition.

If additional actives are added, the serving of softgels may be increased to 2-4 softgels per day to accommodate the increased volume/mass of ingredients.

Example 5: Gummy 1. An oral supplement in the form of a gummy (with sugar) is prepared using the components and parameters shown Table 6 below to give Gummy 1.

TABLE 6
Components of Example 5: Gummy 1

| Ingredient | per gummy (mg) | % | Function |
|---|---|---|---|
| Cane Sugar | 1100 | 30.99 | Gummy Base |
| Tapioca Syrup | 900 | 25.35 | Gummy Base |
| Water | 400 | 11.27 | Solvent |
| Pectin | 300 | 8.45 | Gelling agent |
| Natural Tangerine Flavor | 300 | 8.45 | Flavor |
| Malic acid and/or Citric acid | 200 | 5.63 | Flavor |
| Sodium Citrate | 200 | 5.63 | Stabilizer |
| Fruit and Vegetable juice color | 100 | 2.82 | Color |
| Hemp Extract (cont. CBD, 10 mg) | 50 | 1.41 | Active |
| Total: | 3550 | 100 | |

Alternative gummy bases include glucose syrup, and carriers include gelatin, sugar, fructose, and corn syrup. Pectin from apple or citrus may be used. Typical flavorings include citrus flavor, berry mix flavor, tropical fruit flavor, ginger flavor, tangerine flavor, and tart cherry. Typical colors include carrot juice color, beet juice color, genipap fruit color, spinach color, and artificial FD&C colors.

Example 6: Gummy 2. An oral supplement in the form of a gummy (low added-sugar) is prepared using the components and parameters shown Table 7 below to give Gummy 2.

TABLE 7
Components of Example 6: Gummy 2

| Ingredient | per gummy (mg) | % | Function |
|---|---|---|---|
| Fruit juice concentrate (puree) | 1500 | 53.1 | Gummy Base and Flavor |
| Fruit Pectin | 1000 | 35.4 | Gelling agent |
| Microcrystalline cellulose | 100 | 3.54 | Stabilizer |
| Coconut oil | 100 | 3.54 | Gummy Base |
| Yellow Beeswax | 70 | 2.48 | Stabilizer |
| Hemp Extract (cont. CBD, 10 mg) | 50 | 1.77 | Active |
| Carnauba wax | 5 | 0.18 | Coating |
| Total: | 2825 | 100 | |

Alternative base (mixture) includes apple juice (conc.), apple puree (conc.), strawberry puree, raspberry puree, blackberry puree, mango puree (conc.), peach puree (conc.), blackcurrant juice (conc.), blackberry juice (conc.), purple carrot juice (conc.).

The gummies of Examples 3-4 are administered to a subject orally, via ingestion (i.e., chewing) of one gummy as needed.

In each gummy, a typical CBD content is 5-25 mg. Specific examples include 5-10 mg CBD (ea.).

Example 7: Liquid Capsule 1. An oral supplement in the form of a liquid capsule is prepared using the components and parameters shown Table 8 below to give Liquid Capsule 1.

TABLE 8
Components of Example 7: Liquid Capsule 1

| Ingredient | per capsule (mg) | % | Function |
|---|---|---|---|
| MCT oil | 300 | 58.25 | Carrier |
| Cellulose + water | 150 | 29.13 | Capsule shell |
| Hemp extract (cont. CBD, 10 mg) | 60 | 11.65 | Active |
| Rosemary extract | 5 | 0.97 | Antioxidant |
| Total: | 515 | 100 | |

The liquid capsule of Example 7 is administered to a subject (e.g. an adult) orally, via ingestion of one capsule per day (e.g. with water).

In each softgel, a typical CBD content is 5-20 mg.

Example 8: Powder 1, Joint-Health Type. An oral supplement in the form of a drink-mix powder for joint health is prepared using the components and parameters shown Table 9 below to give Powder 1.

TABLE 9
Components of Example 8: Powder 1

| Ingredient | per scoop (mg) | % | Function |
|---|---|---|---|
| Chicken bone Broth (or Hydrolyzed | 5000 | 90.09 | Carrier/Active |
| collagen) + other collagen sources | | | (collagen source) |
| (fish collagen, eggshell collagen, etc.) | | | |
| Ntural or Artificial Flavors | 230 | 4.14 | Flavor |
| (Citrus, Pineapple) | | | |
| Citric Acid | 150 | 2.70 | Acidic Flavor |
| Hemp Extract (cont. CBD, 10%) | 100 | 1.80 | Active |
| Stevia extract | 30 | 0.54 | Sweetener |
| Lecithin (Sunflower or Soy) | 30 | 0.54 | Emulsifier |
| Silicon dioxide | 10 | 0.18 | Anti-caking agent |
| Total: | 5550 | 100 | |

The oral supplement of Example 8 is administered to a subject via ingestion of a drink (once daily) prepared via the following directions/protocol:

1. Mix one (1) scoop of Powder 1 into 4 to 8 oz. of liquid beverage (e.g. water, milk, fruit juice, etc.) in a cup, blender, or shaker bottle;

### 2. Stir/blend/shake to mix.

A standard serving of

Example 9: Powder 2, Keto-Diet Type. An oral supplement in the form of a drink-mix powder for keto diets is prepared using the components and parameters shown Table 10 below to give Powder 1.

TABLE 10
Components of Example 9: Powder 2

| Ingredient | per scoop (mg) | % | Function |
|---|---|---|---|
| MCT powder (coconut) | 3000 | 92.31 | Keto-powder-Active |
| Soy Lecithin | 150 | 4.62 | Emulsifier |
| Hemp Extract (cont. CBD, 10%) | 100 | 3.08 | Active |
| Total: | 3250 | 100 | |

The oral supplement of Example 9 is administered to a subject via ingestion of a drink (once daily) prepared via the following directions/protocol:

1. Mix one (1) scoop of Powder 2 into 6 to 8 oz. of liquid beverage (e.g. water, coffee, smoothie, etc.) in a cup, blender, or shaker bottle;

### 2. Stir/blend/shake to mix.

Alternatively, Powder 2 may be added to a food of choice, such as cereal, oatmeal, cake mix, etc.

Example 10: Powder 3, Drink-Mix Type. An oral supplement in the form of a drink-mix powder for hydration is prepared using the components and parameters shown Table 11 below to give Powder 1.

TABLE 11
Components of Example 10: Powder 3

| Ingredient | per packet (mg) | % | Function |
|---|---|---|---|
| Cane Sugar | 2000 | 42.92 | Carrier/Base |
| Xylitol | 2000 | 42.92 | Carrier/Base |
| Natural or Artificial Flavors | 200 | 4.29 | Flavor |
| (Berry, Lemonade) | | | |
| Gum Arabic | 120 | 2.58 | Stabilizer |
| Citric acid | 100 | 2.15 | Acidic Flavor |
| Malic Acid | 100 | 2.15 | Acidic Flavor |
| Hemp Extract (cont. CBD, 10%) | 100 | 2.15 | Active |
| Stevia extract | 30 | 0.64 | Sweetener |
| Silicon dioxide | 10 | 0.21 | Anti-caking agent |
| Total: | 4660 | 100 | |

The oral supplement of Example 10 is administered to a subject (e.g. adult) via ingestion of a drink (once daily) prepared via the following directions/protocol:

1. Mix one (1) scoop of Powder 3 into 8 to 12 oz. of water in a cup, blender, or shaker bottle;

### 2. Stir/blend/shake to mix.

As the Hemp extract mixes well with fatty-powders, other fatty powders may be used as a carrier and/or adjuvant in the drink-mix powder.

Example 11: Tablet 1, Chewable. An oral supplement in the form of a chewable tablet is prepared using the components and parameters shown Table 12 below to give Tablet 1.

TABLE 12
Components of Example 11: Tablet 1

| Ingredient | per chew tablet (mg) | % | Function |
|---|---|---|---|
| Lactose | 900 | 77.39 | Carrier/Base |
| Natural Flavors | 100 | 8.6 | Flavor |
| MCT Oil Powder | 90 | 7.74 | Carrier/Base |
| Hemp Extract (cont. CBD, 15 mg) | 60 | 5.16 | Active |
| Magnesium Stearate | 10 | 0.86 | Anticaking agent |
| Stevia Extract | 3 | 0.26 | Sweetener |
| Total: | 1163 | 100 | |

Common natural flavors for chewables include various berry flavors (e.g. raspberry, blueberry, etc.), berry mix flavor, tropical fruit flavor, ginger flavor, tangerine flavor, tart cherry, etc. Artificial flavors can vary in taste names (e.g. cotton candy, rainbow, etc.). Citric acid and/or malic acid may be added under flavor component for acidic taste note. Alternative organic acids (e.g. tartaric acid) could also be used as acidifiers.

Example 12: Tablet 2, Chewable. An oral supplement in the form of a chewable tablet is prepared using the components and parameters shown Table 13 below to give Tablet 2.

TABLE 13
Components of Example 12: Tablet 2

| Ingredient | per chew tablet (mg) | % | Function |
|---|---|---|---|
| Mannitol | 410 | 35.53 | Carrier/Base |
| Fructose | 280 | 24.26 | Carrier/Base |
| Xylitol | 200 | 17.33 | Carrier/Base |
| Natural Flavors | 85 | 7.37 | Flavor |
| (Orange, Berry, Citrus) | | | |
| Microcrystalline Cellulose | 80 | 6.93 | Stabilizer |
| Hemp Extract | 50 | 4.33 | Active |
| (cont. CBD, 10 mg) | | | |
| Stearic Acid | 16 | 1.39 | Lubricant |
| Citric Acid, Malic Acid, | 13 | 1.13 | Acidic Flavor |
| Tartaric Acid | | | |
| Magnesium Stearate | 9 | 0.78 | Anti-caking agent |
| Silicon Dioxide | 8 | 0.69 | Anti-caking agent |
| Natural Sweet | 3 | 0.26 | Sweetener/Flavor |
| Flavor Enhancer | | | |
| Total: | 1154 | 100 | |

The tablets of Examples 11-12 are administered to a subject orally, according to the following directions/protocol:

1. Take one tablet by mouth. Tablets can be swallowed, chewed (better), or held in mouth to dissolve before swallowing (best).

Example 13: Tablet 3. An oral supplement in the form of a tablet is prepared using the components and parameters shown Table 14 below to give Tablet 3.

TABLE 14
Components of Example 13: Tablet 3

| Ingredient | per tablet (mg) | % | Function |
|---|---|---|---|
| Microcrystalline Cellulose | 175 | 57.95 | Carrier |
| Hemp Extract (cont. CBD 15 mg) | 60 | 19.87 | Active |
| Corn Starch | 30 | 9.93 | Carrier |
| Stearic Acid | 15 | 4.97 | Lubricant |
| Sodium Croscarmellose | 9 | 2.98 | Disintegrant |
| Silicon Dioxide | 8 | 2.65 | Anti-caking agent |
| Hydroxypropyl Methyl Cellulose | 5 | 1.66 | Coating |
| Total: | 302 | 100 | |

The tablet of Example 13 is administered to a subject orally, according to the following directions/protocol:

1. Swallow one tablet per day with water. Do not chew the tablet.

In each tablet of Examples 11-13, a typical CBD content is −15 mg per tablet.

Example 14: Solid Capsule 1. An oral supplement in the form of a solid capsule is prepared using the components and parameters shown Table 15 below to give Solid Capsule 1.

TABLE 15
Components of Example 14: Solid Capsule 1

| | per capsule | | | |
|---|---|---|---|---|
| Ingredient | (mg) | % | Function | Notes: |
| Microcrystalline | 300 | 62.5 | Carrier | Rice flower is a |
| Cellulose | | | | common natural alternative |
| | | | | to capsule filler instead |
| | | | | of MCC |
| CBD (powdered) | 50 | 10.42 | active | |
| Silicon Dioxide | 10 | 2.08 | Anti-caking | Not always needed |
| | | | agent | |
| Magnesium | 10 | 2.08 | Anti-caking | Not always needed |
| Stearate | | | agent | |
| Hydroxypropyl | 110 | 22.92 | Capsule | Animal based version: |
| Methyl Cellulose | | | shell | Gelatin Capsule shell |
| | | | | (can be Bovine-Halal) |
| Total: | 480 | 100 | | |

The solid capsule of Example 14 is administered to a subject orally, according to the following directions/protocol:

1. Swallow one capsule per day with water. Do not chew the capsule.

In each solid capsule, a typical CBD content is 15-50 mg.

Example 15: Multi-Hemp Extract Composition. A dietary supplement in the form of a multi-hemp extract composition is prepared using the components and parameters shown Table 16 below to give Multi-Hemp Extract Composition 1.

TABLE 16
Components of Example 15: Multi-Hemp Extract Composition 1

| Ingredient | Loading - Percent Range, % |
|---|---|
| Hemp Dehydrate Powder/Flour | 20-50 |
| Hemp Fresh Juice Liquid Concentrate | 20-40 |
| Hemp Ethanol/Aqueous-Ethanol Liquid Extract | 20-30 |
| Carrier | 10-40 |
| Total: | 100 |

The loading of the hemp components is based on total solids, (dry-weight basis).

The hemp dehydrate powder/flour is prepared from Cannabis leafs, buds, and flowers, with the leaf comprising the predominant feedstock by total weight.

The hemp fresh juice liquid concentrate is prepared from aerial portions of immature Cannabis plants.

The hemp ethanol/aqueous-ethanol liquid extract is obtained from solvent extraction of Cannabis buds and flowers, and is utilized as a liquid concentrate in aqueous ethanol and/or a vegetable oil matrix.

The carrier is an excipient, such as maltodextrin, pectin, gum arabic, starch, SiO₂, or combinations thereof.

The Multi-Hemp Extract Composition 1 may comprise one or more additional botanical components for additional nutritional and/or health benefits.

The Multi-Hemp Extract Composition 1 is administered to a subject orally via a daily dosage form such as a tablet form sized appropriately for a 2× tablet daily serving.

Example XO: Further Multi-Hemp Extract Compositions. A dietary supplement in the form of a multi-hemp extract composition is prepared using the components and parameters shown in Table X1 below to given the various Multi-Hemp Extract Composition Formulations 1-5.

Formulations 1-5 are produced via a formulation method. The method typically comprises the step of combining CBD-Hemp Concentrate with water to produce an intermediary composition. In certain embodiments, the total solids are diluted in the CBD-Hemp Concentrate to ˜15.5 wt. % with water. The intermediary composition is then added to a low-shear mixer, which is then turned on. The intermediary composition is then heated to ˜140° F. The high-shear mixer is then turned up to ˜2,000 rpm. Sunflower lecithin is added to the intermediary composition and mixed for 15 minutes. The intermediary composition is cooled to ˜120° F. Vegetable gum, hemp dehydrate, maltodextrin, and silicon dioxide are then slowly added to the intermediary composition while the mixer is still at ˜2,000 rpm. The intermediary composition is then pasteurized and the temperature of the intermediary composition is kept at ˜120-130° F. Corn or pea starch are slowly added to the intermediary composition and mixed well. After the corn/pea starch is incorporated, the temperature is lowered to 100-110° F.

Hemp solvent extract is then heated in medium-chain triglyceride (MCT) oil to 100-110° F. and additional optional ingredients (e.g. Tara gum, xanthan gum, pectin, and/or tapioca starch) are added to the hemp solvent extract. The mixer speed is then increased to ˜2,500 rpm. The hemp solvent extract is then very slowly added to the intermediary composition and is mixed until the intermediary composition forms a uniform emulsion. Mixing generally continues for 20-30 minutes.

The uniform emulsion is then applied to a refractive (or ref ractance) window drying (RWD) belt with a target application temperature of ˜100-120° F. The target moisture of the dry product is generally <3.6 wt. %. Examples are shown in Tables X2 to X5 further below. Following drying of the uniform emulsion, the uniform emulsion is then conveyed to milling operations (e.g. with a 0.0020 Fitz Screen).

TABLE X1
CBD Dietary Supplement Formulation Examples

| | Formulation No. | | | | |
|---|---|---|---|---|---|
| Ingredients (g) | 1 | 2 | 3 | 4 | 5 |
| CBD-Hemp Concentrate | 291 | 291 | 291 | 291 | 291 |
| Hemp Dehydrate | 60.00 | 60.00 | 60.00 | 60.00 | 60.00 |
| Hemp Solvent Extract | 20.00 | 20.00 | 30.00 | 16.40 | 16.9 |
| Folium Hemp Oil* | 0 | 0 | 0 | 0 | 8.10 |
| Robertet Broad Spectrum Hemp Oil** | 0 | 0 | 0 | 8.6 | 0 |
| Water (added) | 195 | 195 | 195 | 195 | 195 |
| Silicon Dioxide | 4.5 | 4.5 | 4.5 | 4.5 | 4.5 |

Carriers:

| | | | | | |
|---|---|---|---|---|---|
| Sunflower Lecithin | 1.2 | 1.2 | 1.2 | 1.2 | 1.2 |
| Gum Arabic | 10 | 10 | 10 | 10 | 10 |
| Corn Starch | 25 | 0 | 0 | 0 | 0 |
| Pea Starch | 0 | 25 | 25 | 25 | 25 |
| Maltodextrin (e.g., Tapioca) | 25 | 25 | 25 | 25 | 25 |
| Total Weight: | 631.7 | 631.7 | 641.7 | 636.7 | 636.7 |

*available from Folium Biosciences
**available from Robertet and Kiersun

The CBD dietary supplement of Formulations 1-5 all comprise 291 g of CBD-Hemp Concentrate. The CBD-Hemp Concentrate is prepared from aerial portions of immature Cannabis. The CBD-Hemp Concentrate is a fresh juice concentrate and is 25.79 wt. % solids by loading.

The hemp dehydrate is prepared from Cannabis leafs, buds, flowers, or combinations thereof. The hemp dehydrate is generally jet-milled, and in some embodiments the hemp dehydrate is jet-milled at maximum speeds.

The hemp solvent extract is prepared from solvent extraction of Cannabis buds and flowers. The hemp solvent extracts of Formulations 1-5 are in medium-chain triglyceride (MCT) oil. The hemp solvent extracts are generally in the CBD dietary supplement in an amount of 9-15 wt. % of total weight loading.

Formulations 1-5 comprise silicon dioxide. In general, the silicon dioxide is powdered and has CAS #: 7631-86-9. Formulations 1-5 may optionally include an enriched and/or purified CBD extract.

Formulations 1-5 further comprise several carriers and/or excipients. Specifically, Formulations 1-5 comprise sunflower lecithin, gum arabic, corn starch, pea starch, and maltodextrin. The maltodextrin of Formulations 1-5 can be obtained from at least one of tapioca, cassava, corn, potato, and rice. The corn starch has CAS #: 9005-25-8. The multi-hemp extract composition may also optionally include one or more other carriers and/or excipients, such as Tara gum, xanthan gum, pectin, modified corn starch, tapioca starch, oyster shell powder, tricalcium phosphate, and/or sugar.

TABLE X2
Refractive Window Drying (RWD) Treatment Examples

| RWD Example No. | X1 | X2 | X3 | X4 |
|---|---|---|---|---|
| Formulation No. | 1 | 1 | 1 | 1 |
| Application Temp. (° F.) | 110 | 100 | 100 | 100 |
| Belt Speed | 12 | 13 | 14 | 13 |
| Powder Moisture (%) | 2.97 | 3.26 | 3.06 | 3.9 |
| Powder Yield (g) | 18 | 34 | 19 | 18 |
| Pasteurized? | No | No | No | No |

TABLE X3
Refractive Window Drying (RWD) Treatment Examples

| RWD Example No. | X5 | X6 | X7 | X8 |
|---|---|---|---|---|
| Formulation No. | 1 | 1 | 1 | 1 |
| Application Temp. (° F.) | 100 | 110 | 82 | 100 |
| Belt Speed | 13 | 14 | 12 | 12 |
| Powder Moisture (%) | 9.13 | 5.42 | 5.22 | 3.27 |
| Powder Yield (g) | 15 | 31 | 17 | 31 |
| Pasteurized? | Yes | Yes | Yes | Yes |
| Starch Added After | 15 g | 20 g | 20 g | 25 g |
| Pasteurizing? | corn | corn | corn | corn |
| | starch | starch | starch | starch |

TABLE X4
Refractive Window Drying (RWD) Treatment Examples

| RWD Example No. | X9 | X10 | X11 | X12 |
|---|---|---|---|---|
| Formulation No. | 1 | 1 | 1 | 2 |
| Application Temp. (° F.) | <100 | 110 | 100 | 110 |
| Belt Speed | 14 | 14 | 13 | 13 |
| Powder Moisture (%) | 3.82 | 3.67 | 3.6 | 2.41 |
| Powder Yield (g) | 22 | 34 | 36 | 23 |
| Pasteurized? | Yes | Yes | Yes | Yes |
| Starch Added After | 25 g | 25 g | 25 g | 25 g |
| Pasteurizing? | corn | corn | corn | pea |
| | starch | starch | starch | starch |

TABLE X5
Refractive Window Drying (RWD) Treatment Examples

| RWD Example No. | X13 | X14 | X15 | X16 |
|---|---|---|---|---|
| Formulation No. | 2 | 3 | 4 | 5 |
| Application Temp. (° F.) | 100 | 110 | 110 | 110 |
| Belt Speed: | 14 | 12 | 12 | 12 |
| Powder Moisture (%) | 2.82 | 2.58 | 2.64 | 3.26 |
| Powder Yield (g) | 38 | 85 | 175 | 174 |
| Pasteurized? | Yes | Yes | Yes | Yes |
| Starch Added After | 25 g | 25 g | 25 g | 25 g |
| Pasteurizing? | pea | pea | pea | pea |
| | starch | starch | starch | starch |

Upon initial review of the end products obtained, Example Nos. X11, X12, X14, X15, and X16 were most preferred, followed by the others.

An exemplary formulation is illustrated in Table X6 below. In further formulations (not shown), each of the components may be varied by about ±50%, about ±25%, about ±10%, or about ±5%.

TABLE X6
Formulation 1 - Solids and Water Weight Loading

| | Weight | Solids | | Weight |
|---|---|---|---|---|
| | Loading | Loading | Water | Percent |
| Ingredients | (g) | (g) | (g) | (%) |
| CBD-Hemp | 291 | 75 | 216 | 34.00 |
| Water (added) | 195 | 0 | 195 | 0.00 |
| Hemp Dehydrate | 60 | 60 | n/a | 27.20 |
| Silicon Dioxide | 4.4 | 4.4 | n/a | 1.99 |
| Hemp Solvent Extract | 20 | 20 | n/a | 9.07 |

Carriers/Excipients:

| | | | | |
|---|---|---|---|---|
| Sunflower Lecithin | 1.2 | 1.2 | n/a | 0.54 |
| Gum Arabic | 10 | 10 | n/a | 4.53 |
| Maltodextrin | 25 | 25 | n/a | 11.33 |
| (e.g., Tapioca) | | | | |
| Pea Starch | 25 | 25 | n/a | 11.33 |
| Total: | 631.6 | 220.6 | | 100.00 |

### Personal Care Products

Example 16: Body Butter. A personal care product in the form of a body butter is prepared using the components and parameters shown Table 17 below.

TABLE 17
Components of Example 16: Body Butter

| Ingredient | Wt. % |
|---|---|
| Water | 58.62 |
| Carbomer | 0.6 |
| Glycereth-26 | 2 |
| Butylene Glycol | 4 |
| Propanediol | 2.5 |
| Chlorphenesin | 0.28 |
| Vegetable Glyceryl | 1 |
| Stearate/PEG-100 Stearate | |
| Glyceryl Stearate | 1 |
| Neopentyl Glycol Dicaprylate/Dicaprate | 10 |
| PEG-4 Diheptanoate | 5 |
| Jojoba Seed Oil | 1 |
| Safflower Seed Oil | 6 |
| Hemp Seed Oil | 1 |
| Cetearyl Alcohol | 2 |
| Tocopheryl Acetate | 0.5 |
| Menthol | 0.15 |
| Aloe Vera Leaf Extract | 1 |
| Phenoxyethanol | 0.75 |
| Full-spectrum Hemp Extract | 0.6 |
| (Cannabis sativa flower extract) | |
| Hotact | 1 |
| Water | 0.5 |
| Green Tea Extract | 0.5 |

Example 17: Gel-Cream. A personal care product in the form of a gel-cream is prepared using the components and parameters shown Table 18 below.

TABLE 18
Components of Example 17: Gel-cream

| Ingredient | Wt. % |
|---|---|
| Water, Purified | 75.905 |
| Acrylates/C10-C30 Alkyl Acrylate Crosspolymer | 0.25 |
| Disodium EDTA | 0.15 |
| Dipotassium Glycyrrhizate | 0.2 |
| Calcium Chloride | 0.05 |
| Sodium PCA | 0.1 |
| BIS-PEG-18 Methyl Ether Dimethyl Silane | 3 |
| Cetearyl Alcohol and Cetearyl Glucoside | 1.8 |
| Propanediol | 3 |
| Chlorphenesin | 0.295 |
| Dimethicone, 5 cst | 7 |
| Arginine | 0.4 |
| Water, Purified | 1.99 |
| Sodium Hyaluronate | 0.01 |
| Allantoin | 0.2 |
| Omega Liposome | 1 |
| Yeast Extract [Prohyal] | 0.5 |
| Mangifera Indica Fruit Extract | 1 |
| Full Spectrum Hemp Extract | 1.1 |
| Cannabis Sativa Seed Oil | 0.5 |
| Water (and) Glycerin (and) Citrullus vulgaris (Watermelon) | 0.5 |
| Fruit Extract (and) Pyrus Malus (Apple) Fruit Extract | |
| (and) Lens Esculenta (Lentil) Fruit Extract | |
| Water (and) Rosmarinus Officinalis | 0.3 |
| (Rosemary) Leaf Extract (and) Lecithin | |
| Sodium Polyacrylate | 0.75 |

Example 18: Lotion. A personal care product in the form of a lotion is prepared using the components and parameters shown Table 19 below.

TABLE 19
Components of Example 18: Lotion

| Ingredient | Wt. % |
|---|---|
| Water | 74.625 |
| Acrylates Crosspolymer | 0.25 |
| Glycerin | 5 |
| Butylene Glycol | 3 |
| Pentylene Glycol | 2 |
| Disodium EDTA | 0.1 |
| Allantoin | 0.2 |
| Dipotassium Glycyrrhizate | 0.02 |
| Panthenol | 0.15 |
| Chlorphenesin | 0.28 |
| Glyceryl Acrylate/Acrylic Acid Colpolymer | 1 |
| (and) Glycerin (and) Propylene Glycol | |
| (and) Water (and) PVM/MA Copolymer | |
| C12-15 Alkyl Benzoate | 2 |
| Isodecyl Neopentanoate | 2 |
| Capric/Caprylic Triglyceride | 3 |
| Full Spectrum Hemp Extract | 1.5 |
| Dimethicone | 3 |
| Sorbitan Laurate | 1 |
| Arginine | 1.125 |

Example 19: Serum. A personal care product in the form of a serum is prepared using the components and parameters shown Table 20 below.

TABLE 20
Components of Example 19: Serum

| Ingredient | Wt. % |
|---|---|
| Water | 86.86 |
| Glycereth-26 | 2 |
| Disodium EDTA | 0.1 |
| Ceteth-20 | 0.12 |
| Acrylates/C10-C30 Alkyl Acrylate Crosspolymer | 0.3 |
| Butylene Glycol | 2 |
| Allantoin | 0.2 |
| Dipotassium Glycyrrhizate | 0.02 |
| Panthenol | 0.1 |
| Phenoxyethanol | 0.5 |
| Methyl Gluceth-20 | 2 |
| Full Spectrum Hemp Extract | 0.5 |
| Bis-PEG-18 Dimethyl Ether Dimethyl Silane | 1 |
| Glyceryl Acrylate/Acrylic Acid Colpolymer | 0.5 |
| (and) Glycerin (and) Propylene Glycol | |
| (and) Water (and) PVM/MA Copolymer | |
| Water (and) Ophiopogon Japonicus Root Extract | 0.5 |
| Triethanolamine | 0.25 |
| Water | 3 |
| Sodium Hyaluronate | 0.05 |

Example 20: Oil. A personal care product in the form of an oil is prepared using the components and parameters shown Table 21 below.

TABLE 21
Components of Example 20: Oil

| Ingredient | Wt. % |
|---|---|
| Olea Europeaea (Olive) Fruit Oil | 65 |
| Macadamia Ternifolia Seed Oil | 23.0267 |
| Limnanthes Alba (Meadowfoam) Seed Oil | 9.9483 |
| Tocopheryl Acetate | 1 |
| Full Spectrum Hemp Extract (~19.5% CBD) | 1.025 |

Example 21: Cream. A personal care product in the form of a cream is prepared using the components and parameters shown Table 22 below.

TABLE 22
Components of Example 21: Cream

| Ingredient | Wt. % |
|---|---|
| Water, Purified | 48.9685 |
| Glycerin | 3 |
| Butylene Glycol | 1 |
| Xanthan Gum | 0.1 |
| Disodium EDTA | 0.15 |
| Dipotassium Glycyrrhizinate | 0.05 |
| Propanediol | 2 |
| Pentylene Glycol | 2 |
| Chlorphenesin | 0.29 |
| Allantoin | 0.2 |
| Ammonium Acryloyldimethyl Taurate/VP | 0.6 |
| Copolymer [Aristoflex AVC] | |
| Polysorbate 80 | 1 |
| Dimethicone, 5 cst | 7.5 |
| Polysilicone-11 (and) Laureth-12 [Gransil Powder] | 1 |
| Capric/Caprylic Triglyceride | 4 |
| Isononyl Isononanoate | 2 |
| Isodecyl Neopentanoate | 4.5 |
| Glyceryl Trioctanoate | 3 |
| Dimethicone, 300 cst | 0.5 |
| Meadowfoam Seed Oil | 1.5 |
| Petrolatum | 1 |
| Stearate | 4 |
| Cetearyl Alcohol (and) Cetereth-20 [Promulgen D] | 4 |
| Butyrosperum Parkii (Shea Butter) | 2 |
| Tocopheryl Acetate | 0.5 |
| Tocopherol | 0.1 |
| Isohexadecane | 2.48 |
| Sodium Hyaluronate (LMW) | 0.005 |
| Sodium Hyaluronate | 0.005 |
| Full Spectrum Hemp Extract (~19.5% CBD) | 2.0515 |
| Phenoxyethanol | 0.5 |

Example 22: Sheet Mask. A personal care product in the form of a sheet mask is prepared using the components and parameters shown Table 23 below.

TABLE 23
Components of Example 22: Sheet Mask

| Ingredient | Wt. % |
|---|---|
| Water | 85.13 |
| Glycerin | 3 |
| Acrylates/C10-C30 Alkyl Acrylate Crosspolymer | 0.12 |
| Disodium EDTA | 0.1 |
| Polysorbate-20 | 0.25 |
| Butylene Glycol | 3 |
| Allantoin | 0.2 |
| Dipotassium Glycyrrhizate | 0.02 |
| Panthenol | 0.1 |
| Phenoxyethanol | 0.35 |
| Methyl Gluceth-20 | 2 |
| Full Spectrum Hemp Extract | 1 |
| Glyceryl Acrylate/Acrylic Acid Colpolymer | 0.5 |
| (and) Glycerin (and) Propylene Glycol (and) | |
| Water (and) PVM/MA Copolymer | |
| Lavender Extract | 0.5 |
| Chamomile Extract | 0.5 |
| Triethanolamine | 0.18 |
| Water | 3 |
| Sodium Hyaluronate | 0.05 |

Example 23: Cooling Spray. A personal care product in the form of a Cooling Spray is prepared using the components and parameters shown Table 24 below.

TABLE 24
Components of Example 23: Cooling Spray

| Ingredient | Wt. % |
|---|---|
| Water | 75.65 |
| Alcohol | 10 |
| Glycerin | 5 |
| Butylene Glycol | 2 |
| Disodium EDTA | 0.1 |
| Ceteth-20 | 2 |
| Allantoin | 0.2 |
| Dipotassium Glycyrrhizate | 0.02 |
| Panthenol | 0.1 |
| Chlorphenesin | 0.28 |
| Phenoxyethanol | 0.35 |
| Full Spectrum Hemp Extract | 1 |
| Peppermint Oil | 0.15 |
| Menthol | 0.1 |
| Water | 3 |
| Sodium Hyaluronate | 0.05 |

Example 24: Stick. A personal care product in the form of a stick is prepared using the components and parameters shown Table 25 below.

TABLE 25
Components of Example 24: Stick

| Ingredient | Wt. % |
|---|---|
| Water | 55.6650 |
| Butylene Glycol | 5 |
| Pentylene Glycol | 5 |
| Sodium Stearate | 15 |
| Citric Acid | 0.4 |
| Xanthan Gum | 0.2 |
| Lecithin | 0.1 |
| Sodium PCA | 0.2 |
| Glycerin | 3 |
| Sodium Hyaluronate (8 g of 1% soln.) | 0.01 |
| Chlorphenesin | 0.29 |
| Bis-Peg-18 Methyl Ether Dimethyl Silane | 7 |
| Oleth-2 | 2 |
| Stearic Acid | 0.5 |
| Tocopheryl Acetate | 0.2 |
| Caprylyl Glycol | 0.1 |
| Chia Seed Oil | 0.4 |
| Retinyl Palmitate | 0.2 |
| Phenoxyethanol | 0.5 |
| Pomegranate Extract | 0.1 |
| Red #33 (0.1% soln.) | 1.135 |
| Matrixyl 3000 | 3 |

Example 25: Lip Balm. A personal care product in the form of a lip balm is prepared using the components and parameters shown Table 26 below.

TABLE 26
Components of Example 25: Lip Balm

| Ingredient | Wt. % |
|---|---|
| Hydrogenated Polyisobutene | 39.2 |
| Petrolatum | 15 |
| Meadowfoam Seed Oil | 10 |
| Ozokerite | 8 |
| Diisostearyl Malate | 6 |
| Shea Butter | 5 |
| Sunflower Seed Oil | 5 |
| Sunflower Seed Wax | 5 |
| Raspberry Seed Oil | 2 |
| Octyldodecanol | 1.5 |
| Hemp Seed Oil | 1.5 |
| Full Spectrum Hemp Extract | 1 |
| Chia Seed Extract | 0.5 |
| Tocopherol | 0.1 |
| Silica | 0.2 |

### Additional Embodiments

The following additional, general embodiments are provided.

Embodiment 1 relates to a composition comprising a cannabis phytocompound.

Embodiment 2 relates to the composition of embodiment 1, wherein the cannabis phytocompound comprises a cannabinoid, a terpenoid, and/or a flavonoid.

Embodiment 3 relates to the composition of embodiment 1 or 2, wherein the cannabis phytocompound comprises cannabidiol (CBD).

Embodiment 4 relates to the composition of embodiment 1, 2, or 3, wherein the composition is further defined as a cannabis extract.

Embodiment 5 relates to a functional composition comprising the composition of any one of embodiments 1-4 and a carrier.

Embodiment 6 relates to the functional composition of embodiment 5, adapted for human consumption or oral administration.

Embodiment 7 relates to the functional composition of embodiment 5, adapted for topical application or administration.

Embodiment 8 relates to a method of preparing the functional composition of any one of embodiments 5-7, comprising combining the composition with the carrier, thereby preparing the functional composition.

Embodiment 9 relates to a method of ameliorating a condition, comprising administering the functional composition of any one of embodiments 5-7 to a subject in an amount effective to elicit a biological response from the subject.

Embodiment 10 relates to the method of embodiment 9, wherein the method comprises administering the functional composition topically to the skin of the subject.

Embodiment 11 relates to the method of embodiment 9, wherein the method comprises administering the functional composition enterally to the subject.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

The terms “comprising” or “comprise” are used herein in their broadest sense to mean and encompass the notions of “including,” “include,” “consist(ing) essentially of,” and “consist(ing) of.” The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-25, ±0-10, ±0-5, or ±0-2.5, % of the numerical values. Further, the term “about” applies to both numerical values when associated with a range of values. Moreover, the term “about” may apply to numerical values even when not explicitly stated.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

What is claimed is:

1. A multi-hemp extract composition comprising:
a first hemp component comprising a hemp dehydrate, where the hemp dehydrate is obtained from at least one of Cannabis leafs, buds, and flowers;
a second hemp component different from the first hemp component, the second hemp component comprising a hemp juice, where the hemp juice is obtained from aerial portions of immature Cannabis plants;
a third hemp component different from the first and second hemp components, the third hemp component comprising a hemp extract, where the hemp extract is obtained from solvent extraction of at least one of Cannabis buds and flowers; and
a carrier component different from each of the hemp components.

2. The multi-hemp extract composition of claim 1, wherein the hemp dehydrate is in the form of a powder or flour.

3. The multi-hemp extract composition of claim 1, wherein the hemp dehydrate is prepared from Cannabis buds.

4. The multi-hemp extract composition of claim 1, wherein the hemp dehydrate is present in an amount of from about 15 to about 50 wt. % on a dry-weight basis of the composition.

5. The multi-hemp extract composition of claim 1, wherein solids of the hemp juice is present in an amount of from about 15 to about 50 wt. % on a dry-weight basis of the composition.

6. The multi-hemp extract composition of claim 1, wherein the hemp extract is present in an amount of from about 5 to about 30 wt. % on a dry-weight basis of the composition.

7. The multi-hemp extract composition of claim 1, wherein the carrier component is present in an amount of from about 10 to about 50 wt. % on a dry-weight basis of the composition.

8. The multi-hemp extract composition of claim 1, wherein the carrier component comprises silicon dioxide (SiO₂).

9. The multi-hemp extract composition of claim 8, wherein the carrier component further comprises at least one selected from the group consisting of sunflower lecithin, maltodextrin, pectin, gum arabic, tara gum, xanthan gum, pectin, corn starch, tapioca starch, pea starch, oyster shell powder, tricalcium phosphate, sugar, and combinations thereof.

10. The multi-hemp extract composition of claim 1, wherein the carrier component comprises at least one selected from the group consisting of sunflower lecithin, maltodextrin, pectin, gum arabic, tara gum, xanthan gum, pectin, corn starch, tapioca starch, pea starch, oyster shell powder, tricalcium phosphate, sugar, and combinations thereof.

11. The multi-hemp extract composition of claim 1, in the form of a powder obtained by drying the composition.

12. The multi-hemp extract composition of claim 11, wherein the composition is dried with a refractive (or refractance) window dryer (RWD).

13. An oral composition for administration to a subject, the oral composition comprising a multi-hemp extract composition, wherein:
the multi-hemp extract composition is according to claim 1; and
optionally, the oral composition is in the form of a tablet or pill.

14. A method of administering a cannabis phytocompound to a subject, the method comprising:
administrating a multi-hemp extract composition to the subject;
wherein the multi-hemp extract composition is according to claim 1.

15. The method of claim 14, wherein the multi-hemp extract composition is orally administered to the subject.
